(19) United States
(12) Patent Application Publication
(10) Pub. No.: US 2024/0285772 A1
(43) Pub. Date: Aug. 29, 2024

(54) PNICTOGEN-CONTAINING HETEROCYCLIC COMPOUNDS AND THEIR USE

(71) Applicants: University of Rhode Island Board of Trustees, Kingston, RI (US); Colgate University, Hamilton, NY (US)

(72) Inventors: Fang Wang, Saunderstown, RI (US); Jacob M. Goldberg, Hamilton, NY (US); Bradley M. Lipka, Middletown, RI (US); Daniel S. Honeycutt, West Warwick, RI (US)

(21) Appl. No.: 18/106,242

(22) Filed: Feb. 6, 2023

Related U.S. Application Data
(60) Provisional application No. 63/324,058, filed on Mar. 26, 2022.

(51) Int. Cl. A61K 47/54; C07D 213/61; C07D 213/64; C07D 213/68; C07D 213/71; C07D 213/76; C07D 235/24; C07D 235/28; C07D 237/12; C07D 239/30; C07D 277/62

(52) U.S. Cl. CPC A61K 47/545; C07D 213/61; C07D 213/64; C07D 213/68; C07D 213/71; C07D 213/76; C07D 235/24; C07D 235/28; C07D 237/12; C07D 239/30; C07D 277/62

(57) ABSTRACT

Disclosed are pnictogen-containing heterocyclic compounds of Formula (I)

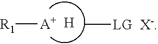

Each of R₁, the heterocyclic ring H, A⁺, LG, and X⁻ is defined herein. Also provided are methods of modifying a substrate using such a compound, conjugated biomolecules thus modified, and pharmaceutical compositions containing one of the conjugated biomolecules.

### CROSS-REFERENCE TO RELATED APPLICATIONS

This application claims the benefit of priority based on U.S. Provisional Application No. 63/324,058, filed on Mar. 26, 2022, the content of which is incorporated herein by reference in its entirety.

### BACKGROUND

Organic reagents are widely used for functionalizing biomolecules and other substrates containing chalcogens, including oxygen, sulfur, and selenium. For example, small molecule-based cysteine bioconjugation technology enables functionalization of proteins and peptides, which significantly expands the chemical space of biomacromolecules. Among various bioconjugation methods, nucleophilic aromatic substitution (S_{N}Ar) of chalcogen-containing residues and the subsequent transformations of these substituted products have been broadly employed.

Despite a broad spectrum of reagents allowing for such transformations, no existing S_{N}Ar reactions are suitable for rapid conjugation of structurally complicated molecules to chalcogens of drugs, biomolecules, and nanomaterials at a second order reaction rate constant greater than 1 M⁻¹·s⁻¹ in neutral aqueous media at an ambient temperature.

Known conventional reagents for chalcogen arylation suffer from three major disadvantages: (1) harsh reaction conditions, (2) slow reaction rates, and (3) limited synthetic modularity. Most known arylation methods are only suitable for relatively simple transformations in a non-aqueous medium at a high concentration. Arylation of chemically labile substrates (e.g., proteins and peptides) in a dilute solution has long been a formidable challenge.

There is a need to develop reagents and methods for arylation of chalcogen-containing substrates, especially biomolecules.

### SUMMARY

To overcome the above-mentioned disadvantages in conventional nucleophilic chalcogen arylation, the present invention provides a novel method for rapid electrophilic chalcogenide arylation using pnictogen-containing heterocyclic electrophiles (CAP), each of which contains at least one positively charged quaternary pnictogen atom (e.g., nitrogen and phosphorus) and at least a leaving group.

A CAP is usually obtained from quaternizing a parental heterocyclic compound and thus converting it from a rather inert, hydrophobic electrophile into a highly reactive, hydrophilic reactant. Not to be bound by any theory, quaternization provides at least two benefits. First, the quaternization of the pnictogen atom introduces a positively charged center to the compound, which increases its polarity and in turn improves its water solubility. Second, the quaternized pnictogen atom increases the affinity of a CAP toward a nucleophilic substrate and thus the reactivity of a CAP as compared to its parent compound. Indeed, high reactivities were observed in all exemplary CAPs shown below.

Further, the prominent electron-withdrawing ability of quaternary pnictogen atoms facilitates effective activation of S_{N}Ar electrophiles without the need of introducing electron-withdrawing groups (EWGs), a strategy frequently adopted by conventional S_{N}Ar-based chalcogen arylations. As such, extra handles can be accommodated on CAPs for further synthetic modifications. In light of the vast availability of substituted heterocyclic compounds, the described pnictogenium-based strategy, as a highly modular toolbox, provides a clear advantage over known nucleophilic chalcogen arylation chemistry.

In addition, CAPs are not only useful for functionalization of chalcogen-containing biomolecules but also applicable to modification of other chalcogen-bearing materials, such as thiol-decorated nanoparticles.

In one aspect, this invention relates to compounds of formula (I):

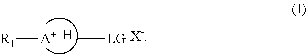

In this formula, R₁ is C₁-C₆ alkyl, C₄-C₁₀ cycloalkyl, 5- to 8-membered heterocycloalkyl, aryl, or heteroaryl;

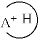

is a heterocyclic ring (i.e., heterocyclic ring H);

A⁺ is a cationic quaternary pnictogen atom, preferably N⁺;

LG is a leaving group selected from the group consisting of halogen, aryloxy, RₐO₂S—, R_{b}O₂S—O—, R_{c}OS—, R_{d}S(O)(NSO₂Rₑ)—, R_{f}S(O)(N⁺(CH₃)₂)—, cyano, nitro, aryloxy, heteroaryloxy, RᵢRₕS⁺—, and RₖRⱼS⁺(O)—, each of Rₐ, R_{b}, R_{c}, R_{d}, Rₑ, R_{f}, Rₕ, Rᵢ, Rⱼ, and Rₖ, independently, being C₁-C₆ alkyl, aryl, heteroaryl, halogen, alkoxy, or aryloxy, preferably being C₁-C₆ alkyl, aryl, or heteroaryl;

X⁻ is a counter anion selected from the group consisting of halide (e.g., F⁻ and I⁻), polyhalide anion, perchlorate, hydroxide, peroxide, siloxide, sulfate, hydrogen sulfate, sulfite, disulfite, dithionate, dithionite, halosulfate, thiosulfate, persulfate, disulfate, sulfinate (R′SO₂⁻), sulfonate (R″SO₃⁻), bis(sulfonyl)imide ((R′SO₂)₂N⁻), nitrate, nitrite, azide, cyanide, cyanate, thiocyanate, phosphate, metaphosphate, polyphosphate, hydrogen phosphate, dihydrogen phosphate, monohalophosphate, dihalophosphate, hexahalophosphate, organophosphonate (R′P(O)(O⁻)₂ or R′P(O)(OR″)(O⁻)), organophosphate (R′OP(O)(O⁻)₂ or R′OP(O)(OR″)(O⁻)), arsenate, alkoxide, alkenoxide, aryloxide, carboxylate, percarboxylate, carbonate, bicarbonate, oxalate, borate, tetrahaloborate, tetraalkylborate, tetraarylborate, carborane, and combinations thereof, each of R′ and R″, independently, being C₁-C₆ alkyl or aryl; and

the number of net negative charges of X⁻ equals to that of the net positive charges of

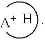

Some compounds of Formula (I) have two, three, four, or more leaving groups on heterocyclic ring H or, independently, on heterocyclic ring H or a different heterocyclic ring attached to heterocyclic ring H.

A subset of the compounds of Formula (I) are compounds of Formula (II):

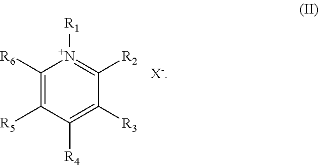

In this formula, R₁ and X⁻ are defined above. Preferably, R₁ is CH₃ or CH₂CH₃ and X⁻ is fluoride, iodide, or tetrafluoroborate.

In Formula (II) above,

the cationic quaternary pnictogen atom is N⁺;

each of R₂, R₃, R₄, R₅, and R₆, independently, is H, F, Cl, Br, I, C₁-C₁₀ alkoxy, RₐO₂S— (e.g., CH₃O₂S—), R_{b}O₂S—O— (e.g., CH₃O₂S—O—), R_{f}S(O)(N⁺(CH₃)₂)— (e.g., phenyl-S(O)(N⁺(CH₃)₂)—), halogenated aryl, halogenated heteroaryl, Het⁺, or -L-Het⁺, in which Het⁺is

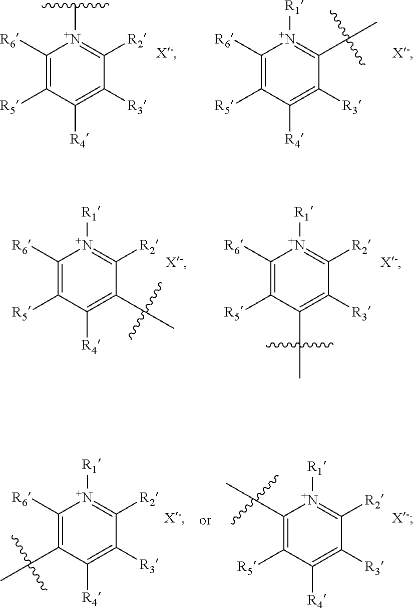

each of Rₐ and R_{b}, independently, is C₁-C₆ alkyl (e.g., CH₃ and CH₂CH₃), or aryl (e.g., phenyl), R₁′ is CH₃ or CH₂CH₃; each of R₂′, R₃′, R₄′, R₅′, and R₆′, independently, is H, NO₂, F, Cl, Br, I, Rₐ′O₂S— (e.g., CH₃O₂S—), R_{b}′O₂S—O— (e.g., CH₃O₂S—O—), or R_{f}S(O)(N⁺(CH₃)₂)— (e.g., phenyl-S(O)(N⁺(CH₃)₂)—); each of Rₐ′, R_{b}′, and R_{f}′, independently, is C₁-C₆ alkyl (e.g., CH₃ and CH₂CH₃) or aryl (e.g., phenyl); and X⁻ is a counter anion selected from the group consisting of halide (e.g., F⁻ and I⁻), polyhalide anion, perchlorate, hydroxide, peroxide, siloxide, sulfate, hydrogen sulfate, sulfite, disulfite, dithionate, dithionite, halosulfate, thiosulfate, persulfate, disulfate, sulfinate (R′SO₂), sulfonate (R″SO₃⁻), bis(sulfonyl)imide ((R′SO₂)₂N⁻), nitrate, nitrite, azide, cyanide, cyanate, thiocyanate, phosphate, metaphosphate, polyphosphate, hydrogen phosphate, dihydrogen phosphate, monohalophosphate, dihalophosphate, hexahalophosphate, organophosphonate (R′P(O)(O⁻)₂ or R′P(O)(OR″)(O⁻)), organophosphate (R′OP(O)(O⁻)₂ or R′OP(O)(OR″)(O⁻)), arsenate, alkoxide, alkenoxide, aryloxide, carboxylate, percarboxylate, carbonate, bicarbonate, oxalate, borate, tetrahaloborate, tetraalkylborate, tetraarylborate, carborane, and combinations thereof, each of R′ and R″, independently, being C₁-C₆ alkyl or aryl; L is a linker including C₁-C₂₀ alkyl, C₂-C₂₀ alkenyl, C₂-C₂₀ alkynyl, alkyl ether or polyether, aryl ether or polyether, oxygen, sulfur, sulfoxide, sulfone, aryl, heteroaryl, alkyl amino, and aryl amino with specific examples selected from the group consisting of

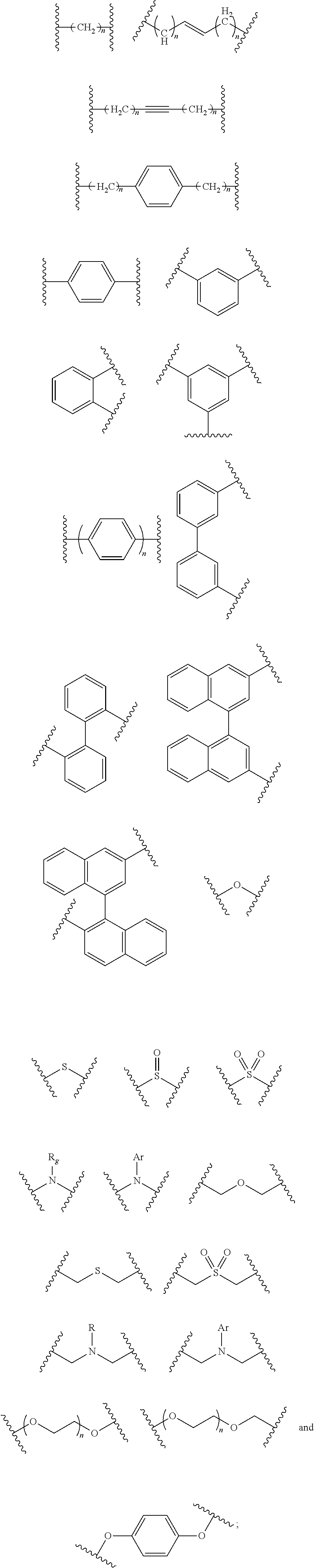

n is an integer from 0 to 20; R_{g} is C₁-C₆ alkyl; and Ar is aryl;

at least one of R₂, R₃, R₄, R₅, and R₆ is the leaving group that is F, Cl, Br, I, RₐO₂S— (e.g., CH₃O₂S—), or R_{b}O₂S—O— (e.g., CH₃O₂S—O—);

when R₂ is F, (i) R₄ is F, Cl, Br, I, C₁-C₁₀ alkoxy, RₐO₂S— (e.g., CH₃O₂S—), or R_{b}O₂S—O— (e.g., CH₃O₂S—O—), (ii) R₅ is NO₂, or (iii) one and only one of R₃, R₄, R₅, and R₆ is Het⁺;

when R₂ is Cl, R₅ is H or Het⁺; and

when R₄ is —SO₂CH₃, (i) R₂ is F, Cl, Br, I, C₁-C₁₀ alkoxy, RₐO₂S— (e.g., CH₃O₂S—), or R_{b}O₂S—O— (e.g., CH₃O₂S—O—), or (ii) one and only one of R₂, R₃, R₅, and R₆ is Het⁺.

Another subset of the compounds of Formula (I) are compounds of Formula (III):

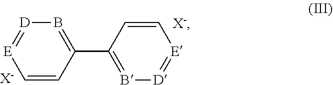

in which

one and only one of B, D, and E is N⁺CH₃ or N⁺CH₂CH₃;

one and only one of B, D, and E is CR₂, R₂ being F, Cl, Br, I, RₐO₂S— (e.g., CH₃O₂S—), or R_{b}O₂S—O— (e.g., CH₃O₂S—O—) as a leaving group;

one and only one of B, D, and E is CR₁₁ (e.g., CH), R₁₁ being C₁-C₆ alkyl, C₁-C₆ alkoxy, C₂-C₆ alkenyl, C₂-C₆ alkynyl, C₁-C₆ alkyl amino, C₄-C₁₀ cycloalkyl, C₄-C₁₀ cycloalkoxy, 5- to 8-membered heterocycloalkyl, 5- to 8-membered heterocycloalkoxy, aryl, aryloxy, heteroaryl, or heteroaryloxy;

one and only one of B′, D′, and E′ is N⁺CH₃ or N⁺CH₂CH₃;

one and only one of B′, D′, and E′ is CR₂′, R₂′ being F, Cl, Br, I, Rₐ′O₂S— (e.g., CH₃O₂S—), or R_{b}′O₂S—O— (e.g., CH₃O₂S—O—) as a leaving group, and each of Rₐ′ and R_{b}′, independently, being aryl (e.g., phenyl) or C₁-C₆ alkyl (e.g., CH₃ and CH₂CH₃); one and only one of B′, D′, and E′ is CR₁₁′ (e.g., CH), R₁₁′ being C₁-C₆ alkyl, C₁-C₆ alkoxy, C₂-C₆ alkenyl, C₂-C₆ alkynyl, C₁-C₆ alkyl amino, C₄-C₁₀ cycloalkyl, C₄-C₁₀ cycloalkoxy, 5- to 8-membered heterocycloalkyl, 5- to 8-membered heterocycloalkoxy, aryl, aryloxy, heteroaryl, or heteroaryloxy;

X⁻ is defined above; and

X′⁻ is a counter anion selected from the group consisting of halide (e.g., F⁻ and I⁻), polyhalide anion, perchlorate, hydroxide, peroxide, siloxide, sulfate, hydrogen sulfate, sulfite, disulfite, dithionate, dithionite, halosulfate, thiosulfate, persulfate, disulfate, sulfinate (R′SO₂⁻), sulfonate (R″SO₃⁻), bis(sulfonyl)imide ((R′SO₂)₂N⁻), nitrate, nitrite, azide, cyanide, cyanate, thiocyanate, phosphate, metaphosphate, polyphosphate, hydrogen phosphate, dihydrogen phosphate, monohalophosphate, dihalophosphate, hexahalophosphate, organophosphonate (R′P(O)(O⁻)₂ or R′P(O)(OR″)(O⁻)), organophosphate (R′OP(O)(O⁻)₂ or R′OP(O)(OR″)(O⁻)), arsenate, alkoxide, alkenoxide, aryloxide, carboxylate, percarboxylate, carbonate, bicarbonate, oxalate, borate, tetrahaloborate, tetraalkylborate, tetraarylborate, carborane, and combinations thereof, each of R′ and R″, independently, being C₁-C₆ alkyl or aryl.

A further subset of the compounds of Formula (I) are compounds of Formula (IV):

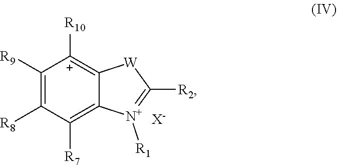

in which

R₁ is CH₃ or CH₂CH₃;

R₂, a leaving group, is RₐO₂S— (e.g., CH₃O₂S—), or R_{b}O₂S—O— (e.g., CH₃O₂S—O—);

each of R₇, R₅, R₉, and R₁₀, independently, is H or C₁-C₆ alkyl; and

W is S or NR₁₁, R₁₁ being C₁-C₆ alkyl.

In this formula, R₁ and X⁻ are defined above.

Still another subset of the compounds of Formula (I) are compounds of Formula (V):

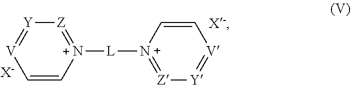

in which

one and only one of V, Y, and Z is CR₂, R₂ being F, Cl, Br, I, RₐO₂S— (e.g., CH₃O₂S—), or R_{b}O₂S—O— (e.g., CH₃O₂S—O—) as a leaving group;

the other two of V, Y, and Z is CR₁₁′ (e.g., CH), R₁₁′ being C₁-C₆ alkyl, C₁-C₆ alkoxy, C₂-C₆ alkenyl, C₂-C₆ alkynyl, C₁-C₆ alkyl amino, C₄-C₁₀ cycloalkyl, C₄-C₁₀ cycloalkoxy, 5- to 8-membered heterocycloalkyl, 5- to 8-membered heterocycloalkoxy, aryl, aryloxy, heteroaryl, or heteroaryloxy;

one and only one of V′, Y′, and Z′ is CR₂′, R₂′ being F, Cl, Br, I, Rₐ′O₂S— (e.g., CH₃O₂S—), or R_{b}′O₂S—O— (e.g., CH₃O₂S—O—) as a leaving group;

the other two of V′, Y′, and Z′ is CR₁₁′ (e.g., CH), R₁₁′ being C₁-C₆ alkyl, C₁-C₆ alkoxy, C₂-C₆ alkenyl, C₂-C₆ alkynyl, C₁-C₆ alkyl amino, C₄-C₁₀ cycloalkyl, C₄-C₁₀ cycloalkoxy, 5- to 8-membered heterocycloalkyl, 5- to 8-membered heterocycloalkoxy, aryl, aryloxy, heteroaryl, or heteroaryloxy;

L is a linker as defined above; and

X⁻ and X⁻ are counter anions as defined above.

The compounds described above can have one or any combinations of the following features:
(i) R₁ is CH₃ or CH₂CH₃;
(ii) X⁻ is fluoride, iodide, or tetrafluoroborate;
(iii) X′⁻ is fluoride, iodide, or tetrafluoroborate;
(iv) in Formula (II), at least one of R₂, R₄, and R₆ is the leaving group; in Formulas (III) and (V), R₂ and R₂′ are the leaving groups; and in Formula (IV), R₂ is the leaving group;
(v) in Formula (II), R₂, being the leaving group, is CH₃O₂S—, R₄ is H, C₁-C₁₀ alkoxy, or Het⁺, and/or R₃ or R₅ is NO₂; and
(vi) in Formula (II), at most only one of R₂, R₃, R₄, R₅, and R₆ is Het⁺ or -L-Het⁺.

Each of alkyl, alkoxy, alkenyl, alkynyl, alkyl amino, cycloalkyl, heterocycloalkyl, heterocyclic ring, phenyl, aryl, aryloxy, heteroaryl, and heteroaryloxy is optionally substituted with one or more of deuterium, halogen (e.g., F), CN, oxo, C₁-C₆ alkyl, C₃-C₆ cycloalkyl, C₁-C₆ haloalkyl, C₂-C₆ alkenyl, C₂-C₆ alkynyl, —OR′, —C(O)R′, —C(O)OR′, —O—COR′, —C(O)NR′R″, —NR′R″, —SR′, —SOR′, —S(O)₂R′, or —POR′R″, in which each of R′ and R″, independently, is H, halogen, C₁-C₆ alkyl, C₁-C₆ haloalkyl, C₃-C₆ cycloalkyl, 3-6 membered heterocycloalkyl, 5-10 membered heteroaryl, or 6 or 10 membered aryl.

Another aspect of this invention relates to a method for modifying a substrate. The method includes the steps of a) providing a substrate, and b) contacting the substrate with a pnictogen-containing heterocyclic compound as described above, thereby obtaining a conjugated substrate containing a substrate moiety derived from the substrate and a pnictogen-containing heterocyclic moiety derived from the pnictogen-containing heterocyclic compound. The substrate preferably contains a functional group selected from the group consisting of hydroxyl, thiol, selenol, and tellurol, and the substrate moiety is linked to the pnictogen-containing heterocyclic moiety via a covalent bond formed by replacing the leaving group in the pnictogen-containing compound with the 0, S, Se, or Te atom in the functional group of the substrate.

In a preferred method, the pnictogen-containing heterocyclic compound is a compound of Formula (II):

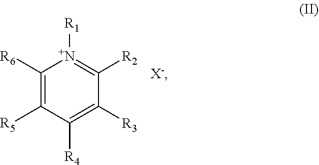

in which

R₁ is CH₃, CH₂CH₃, or -L-Het⁺;

each of R₂, R₃, R₄, R₅, and R₆, independently, is H, F, Cl, Br, I, C₁-C₁₀ alkoxy, RₐO₂S— (e.g., CH₃O₂S—), or R_{b}O₂S—O— (e.g., CH₃O₂S—O—), Het⁺, or -L-Het⁺, in which Rₐ, R_{b}, L, and Het⁺ are as defined above; and

at least one of R₂, R₃, R₄, R₅, and R₆ is the leaving group that is F, Cl, Br, I, RₐO₂S— (e.g., CH₃O₂S—), or R_{b}O₂S—O— (e.g., CH₃O₂S—O—).

The method described above can have one or any combination of the following features:
(i) at most only one of R2, R3, R4, R5, and R6 is Het⁺ or -L-Het⁺,
(ii) the contacting step is performed in a medium,
(iii) the medium is water,
(iv) the contacting step is performed in an aqueous medium having a pH of 2 to 10 (preferably, 3 to 9, and more preferably, 6 to 8), and
(v) the substrate is a biomolecule selected from the group consisting of an amino acid, a peptide, a protein, a deoxyribonucleic acid or a derivative thereof, a ribonucleic acid or a derivative thereof, a peptoid, and poly(amino acid).

Also within the scope of this invention is a conjugated biomolecule comprising (i) a biomolecule moiety derived from a biomolecule, and (ii) a pnictogen-containing heterocyclic moiety derived from any pnictogen-containing heterocyclic compound described above, in which the biomolecule is an amino acid, a peptide, a protein, a deoxyribonucleic acid or a derivative thereof, a ribonucleic acid or a derivative thereof, a peptoid, or poly(amino acid), each having a functional group selected from the group consisting of hydroxyl, thiol, selenol, and tellurol, and the biomolecule moiety is linked to the pnictogen-containing heterocyclic moiety via a covalent bond formed by substitution of the leaving group in the pnictogen-containing heterocyclic compound by the functional group in the biomolecule.

The conjugated biomolecule can further contain a therapeutic moiety derived from a therapeutic agent selected from the group consisting of an anticancer agent, a therapeutically active peptide and protein, a medication for a neurological disorder, a molecular scaffold for targeted drug delivery, or a radioactive tracer, in which the therapeutic moiety is linked to the pnictogen-containing heterocyclic moiety via a covalent bond. Preferably, the therapeutic agent contains a functional group selected from the group consisting of hydroxyl, thiol, selenol, and tellurol, and the pnictogen-containing heterocyclic compound has two leaving groups.

Still within the scope of this invention is a pharmaceutical composition containing any of the conjugated biomolecule described above and a pharmaceutically acceptable carrier thereof. The conjugated biomolecule is present at an effective amount for treating a condition or disorder in a subject.

Table 1 below shows 31 exemplary CAP compounds of the present invention, namely, CAP1-a, CAP1-b, CAP1-F, CAP1-N₃, CAP1-CCH, and CAP2-CAP27.

TABLE 1

| 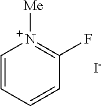 | 1-a |
|---|---|
| 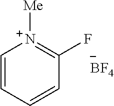 | 1-b |
| 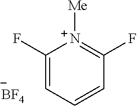 | 1-F |
| 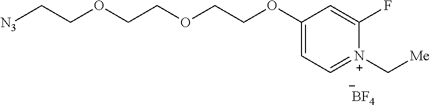 | 1-N₃ |
| 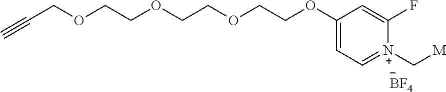 | 1-CCH |
| 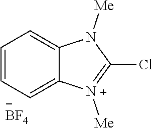 | 2 |
| 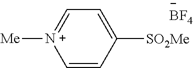 [SMILES: C[n+]1ccc(S(C)(=O)=O)cc1 ; "BF4"] | 3 |
| 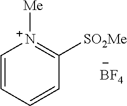 | 4 |
| 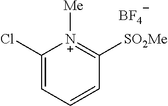 | 5 |
| 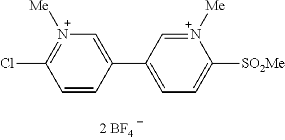 [SMILES: C[n+]1cc(-c2ccc(S(C)(=O)=O)[n+](C)c2)ccc1Cl] | 6 |
| 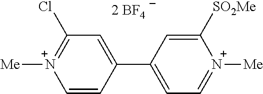 [SMILES: C[n+]1ccc(-c2cc[n+](C)c(S(C)(=O)=O)c2)cc1Cl] | 7 |
| 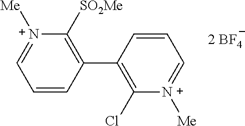 [SMILES: C[n+]1cccc(-c2ccc[n+](C)c2S(C)(=O)=O)c1Cl] | 8 |
| 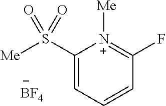 | 9 |
| 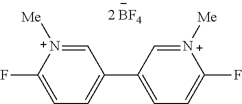 [SMILES: C[n+]1cc(-c2ccc(F)[n+](C)c2)ccc1F] | 10 |
| 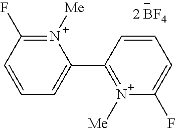 [SMILES: C[n+]1c(F)cccc1-c1cccc(F)[n+]1C] | 11 |
| 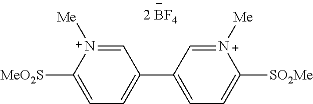 [SMILES: C[n+]1cc(-c2ccc(S(C)(=O)=O)[n+](C)c2)ccc1S(C)(=O)=O] | 12 |
| 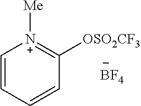 | 13 |
| 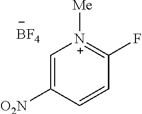 [SMILES: "BF4" ; C[n+]1cc([N+](=O)[O-])ccc1F] | 14 |
| 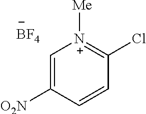 [SMILES: "BF4" ; C[n+]1cc([N+](=O)[O-])ccc1Cl] | 15 |
| 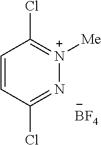 | 16 |
| 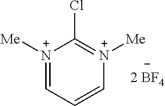 | 17 |
| 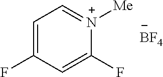 | 18 |
| 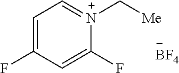 | 19 |
| 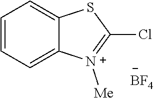 | 20 |
| 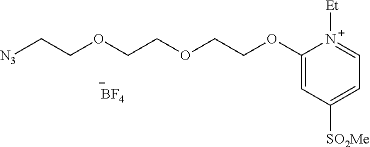 | 21 |
| 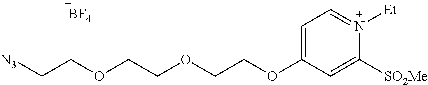 [SMILES: CC[n+]1ccc(OCCOCCOCCN=[N+]=[N-])cc1S(C)(=O)=O ; "BF4"] | 22 |
| 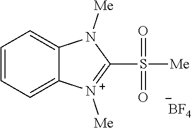 | 23 |
| 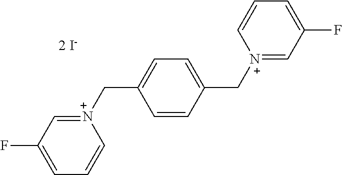 [SMILES: Fc1ccc[n+](Cc2ccc(C[n+]3cccc(F)c3)cc2)c1] | 24 |
| 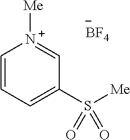 | 25 |
| 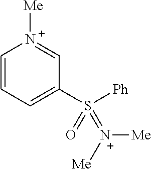 [SMILES: C[N+](C)=S(=O)(c1ccccc1)c1ccc[n+](C)c1] | 26 |
| 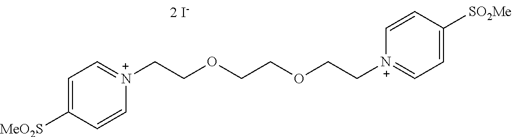 [SMILES: CS(=O)(=O)c1cc[n+](CCOCCOCC[n+]2ccc(S(C)(=O)=O)cc2)cc1] | 27 |

Preferred compounds include Compounds 1-N3, 1-CCH, 4-14, 18, 19, and 21-27.

More preferred compounds are Compounds 4-9, 21, and 22.

The term “chalcogen” herein refers a chemical element in group 16 of the periodic table including oxygen (O), sulfur (S), selenium (Se), and Tellurium (Te).

The term “pnictogen” refers to a chemical element in group 15 of the periodic table including nitrogen (N), phosphorus (P), arsenic (As), antimony (Sb), bismuth (Bi), and moscovium (Mc).

The term “halogen” herein refers to a fluoro (F), chloro (Cl), bromo Br), or iodo (I) radical. A particular halogen is a fluoro radical.

The term “alkyl” refers to a straight or branched hydrocarbon group, containing 1-20 carbon atoms (e.g., C₁₋₁₀, C₁₋₆, C₁₋₄, and C₁₋₃) and a monovalent radical center derived by the removal of a hydrogen atom from a carbon atom of a parent alkane. Exemplary alkyl groups are methyl, ethyl, n-propyl, isopropyl, n-butyl, iso-butyl, tert-butyl, n-pentyl, and n-hexyl. “Alkyl” includes “haloalkyl”, which refers to alkyl substituted with one or more halogens (fluoro, chloro, bromo, or iodo). Examples include fluoromethyl, difluoromethyl, trifluoromethyl, fluoroethyl (e.g., 1-fluoroetyl and 2-fluoroethyl), difluoroethyl (e.g., 1,1-, 1,2-, and 2,2-difluoroethyl), and trifluoroethyl (e.g., 2,2,2-trifluoroethyl).

The term “alkoxy” refers to an —O-alkyl group. The terms “alkoxy” and “alkyl ether” are used interchangeably. Examples are methoxy, ethoxy, propoxy, and isopropoxy. Alkoxy also includes haloalkoxy, namely, alkoxy substituted with one or more halogens, e.g., —O—CH₂Cl and —O—CHClCH₂Cl.

The term “cycloalkyl” refers to a nonaromatic, saturated or unsaturated monocyclic, bicyclic, tricyclic, or tetracyclic hydrocarbon group containing 3 to 12 carbons (e.g., C₃₋₁₀, C₃₋₈, C₄₋₇, and C₃₋₆). Cycloalkyl also includes fused, bridged, and spiro ring systems. Examples are cyclopropyl, cyclobutyl, cyclopentyl, cyclopentenyl, cyclohexyl, cyclohexenyl, cycloheptyl, cyclooctyl, bicyclo[1.1.1]pentyl, bicyclo[2.1.1]hexyl, bicyclo[2.1.1]heptyl, bicyclo[2.2.2]octanyl, and decahydronaphthalene. The term “cycloalkoxy” refers to —O— cycloalkyl.

The term “heterocycloalkyl” refers to a nonaromatic, saturated or unsaturated, 3-8 membered monocyclic, 8-12 membered bicyclic, or 11-14 membered tricyclic ring system having one or more heteroatoms (e.g., O, N, P, and S). The term also includes fused, bridged, and spiro ring systems. Examples include aziridinyl, azetidinyl, pyrrolidinyl, dihydrofuranyl, tetrahydrofuranyl, tetrahydropyranyl, tetrahydrothiophenyl, tetrahydro-2-H-thiopyran-1,1-dioxidyl, piperazinyl, piperidinyl, morpholinyl, imidazolidinyl, azepanyl, dihydrothiadiazolyl, dioxanyl, 2-azaspiro[3.3]heptanyl, quinuclidinyl, and 8-azabicyclo[3.2.1]octanyl. The term “heterocycloalkoxy” refers to an —O-heterocycloalkyl group.

The term “alkenyl” refers to a straight or branched, monovalent, unsaturated aliphatic chain having 2 to 20 carbon atoms (e.g., C₂₋₄, C₂₋₆, and C₂₋₁₀) and one or more carbon-carbon double bonds. Examples are ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, and 2-pentenyl. The term “alkenylene” refers to a straight or branched, bivalent, unsaturated aliphatic chain having 2 to 20 carbon atoms (e.g., C₂₋₄, C₂₋₆, and C₂ ) and one or more carbon-carbon double bonds.

The term “alkynyl” refers to a straight or branched aliphatic chain having 2 to 20 carbon atoms (e.g., C₂₋₄, C₂₋₆, and C₂ ) and one or more carbon-carbon triple bonds. Examples are ethynyl, 2-propynyl, 2-butynyl, 3-methylbutynyl, and 1-pentynyl. The term “alkynylene” refers to a straight or branched, bivalent, unsaturated aliphatic chain having 2 to 20 carbon atoms (e.g., C₂₋₄, C₂₋₆, and C₂ ) and one or more carbon-carbon triple bonds.

The term “aryl” refers a 6-carbon monocyclic, 10-carbon bicyclic, 14-carbon tricyclic aromatic ring system wherein each ring can have one or more (e.g., 1 to 10, 1 to 5, and 1 to 3) substituents. Examples include phenyl, biphenyl, 1- or 2-naphthyl, 1,2-dihydronaphthyl, 1,2,3,4-tetrahydronaphthyl, indenyl, and indanyl. The term “aryloxy” refers to —O-aryl. The term “halogenated aryl” refers to aryl substituted with one, two, three, or more halogen atoms. Examples include 2-fluorophenyl, 3-fluorophenyl, and 4-fluorophenyl.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include pyridinyl, pyridinium, pyrimidinyl, quinolinyl, isoquinolinyl, indolyl, benzimidazolyl, benzoxazolyl, benzothiophenyl, benzofuranyl, pyrazolyl, triazolyl, oxazolyl, thiadiazolyl, tetrazolyl, oxazolyl, isoxazolyl, carbazolyl, furyl, imidazolyl, thienyl, quinolinyl, indolyl, thiazolyl, and benzothiazolyl. The term “heteroaryloxy” refers to —O-heteroaryl. The term “halogenated heteroaryl” refers to heteroaryl substituted with one, two, three, or more halogen atoms.

The term “heterocyclic” includes heterocycloalkyl and heteroaryl.

Alkyl, alkylene, haloalkylene, alkoxyl, cycloalkyl, heterocycloalkyl, heterocycloalkoxy, alkenyl, alkynyl, aryl, and heteroaryl mentioned herein include both substituted and unsubstituted moieties. Examples of a substituent include deuterium, halogen (e.g., F, Cl, and Br), amino, hydroxy, alkyl and haloalkyl (e.g., methyl, fluoromethyl, difluoromethyl, trifluoromethyl, difluoroethyl, and 1,1-difluoro-2-hydroxylethan-1-yl), alkenyl and haloalkenyl (e.g., ethylenyl and 3,3-difluoro-2-methylpropen-3-yl), cycloalkyl (e.g., cyclopropyl and cyclobutyl), cycloheteroalkyl (e.g., tetrahydrofuranyl), —CN, —CONR′R″, —NR′R″, —NR′COR″, —NR′SO₂R″, —NCOOR′, —COR′, —COOR′, —SR′, —SONR′R″, and —OR′, wherein R‘ and R’ are on each occurrence independently selected from the group consisting of hydrogen, C₁₋₆ alkyl, C₂₄ alkenyl, and C₂₄ alkynyl optionally substituted with 1-3 halogens. All substituents can be further substituted.

The term “compound”, when referring to a compound of this invention, also includes its salts, solvates, hydrates, polymorph, co-crystals, tautomers, stereoisomers, or isotopically labeled derivatives thereof.

The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, acetonitrile, dimethylformamide, dimethyl sulfoxide, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereomeric mixture, a cis-isomer, or a trans-isomer. Compounds of such isomeric forms are within the scope of this invention. They can be present as a mixture or can be isolated using chiral synthesis or chiral separation technologies.

“An effective amount” refers to the amount of a compound that is required to confer the therapeutic effect. Effective doses will vary, as recognized by those skilled in the art, depending on the types of symptoms treated, route of administration, excipient usage, and the possibility of co-usage with other therapeutic treatment.

The term “subject” refers to an animal such as a mammal including a human. A human is a preferred subject.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples include colloidal silicon oxide, magnesium stearate, cellulose, sodium lauryl sulfate, and D&C Yellow #10.

The details of one or more embodiments of the invention are set forth in the description below. Other features, objects, and advantages of the invention will be apparent from the description and from the claims.

### DETAILED DESCRIPTION

The pnictogen-containing heterocyclic compounds of this invention contains at least one cationic quaternary pnictogen atom and a leaving group.

The compounds are particularly useful in modifying a substrate having a functional group such as hydroxyl (OH), thiol (SH), selenol (SeH), and tellurol (TeH). These functional group in the substrate can substitute the leaving group of the pnictogen-containing heterocyclic compound. It is unexpectedly found that reaction rate of the modification is surprisingly fast and that the reaction can be carried out in a mild condition such as in an aqueous medium at a mild pH range in the absence of a catalyst. The pnictogen-containing heterocyclic compounds of this invention each have a high water solubility. They can be used to modify a substrate in a medium with a high water content, which is not achievable with other existing reagents due to their low aqueous solubility or instability. Modification is readily carried out under mild conditions near pH 7 without the need to use a base, which is not practically possible with other existing reagents due to their slow reaction with chalcogens. As shown in examples below, the compounds of this invention allow incorporation of biomolecules with high structural complexity, which cannot be attained by other existing methods due to limited structural complexity of the employed reagents.

Suitable substrates include pharmaceutical active ingredients, amino acids, peptides, proteins, polymers, modified DNA, modified RNA, polymers, other macromolecules, and nanoparticle.

Referring to Formula (I):

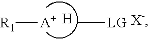

R₁ can be hydrogen, oxygen, alkyl, aryl, amino, alkylamino, arylamino, dialkylamino, diarylamino, borate (RR′R″B⁻—), or silyl (—SiRR′R″), in which each of R, R′, and R″, independently, is hydrogen, alkyl, aryl, halogen, hydroxy, alkoxy, aryloxy, siloxy, amino, alkylamino, arylamino, dialkylamino, or diarylamino. LG is a leaving group, which is replaced by the incoming sulfur nucleophile during a modification reaction. It can be selected from group consisting of hydrogen, halogen, cyano, thiolate (RS—), sulfoxide (R(O)S—), sulfonyl (R(O)₂S—), sulfonate (R(O)₂S—O—), sulfonamide, diazo, azido, nitro, nitroso, hydroxy, alkoxy, alkenoxy, aryloxy, carboxyl (RCOO—), siloxy, phosphonate ((RO)(R′O)(O)P—), phosphate ((RO)(R′O)(O)P—O—), trialkyl ammonium, triaryl ammonium, trialkyl phosphonium, and triaryl phosphonium, in which R and R′ are independently selected from the group consisting of hydrogen, alkyl, aryl, halogen, hydroxy, alkoxy, aryloxy, siloxy, amino, alkylamino, arylamino, dialkylamino, and diarylamino.

The heterocyclic compounds can be any of the compounds of the following formula:

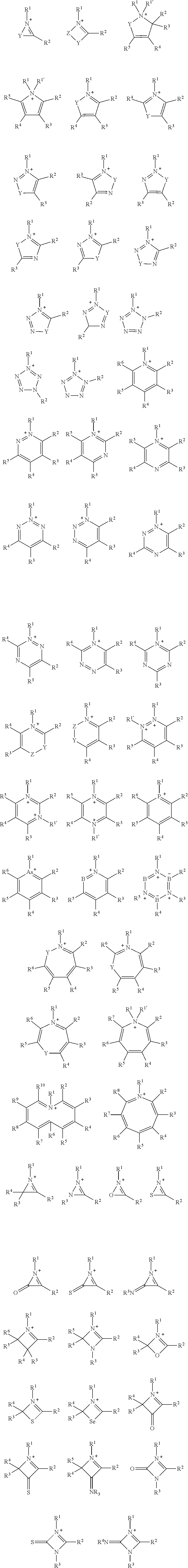

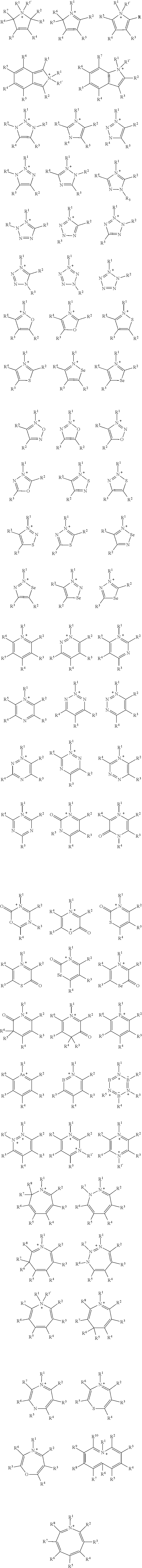

In the above formulas, R¹ and R¹′ are independently selected from the group consisting of hydrogen, oxygen, alkyl, aryl, amino, alkylamino, arylamino, dialkylamino, diarylamino, borate (RR′R″B⁻—), and silyl (—SiRR′R″), in which R, R′, and R″ are independently selected from the group consisting of hydrogen, alkyl, aryl, halogen, hydroxy, alkoxy, aryloxy, siloxy, amino, alkylamino, arylamino, dialkylamino, and diarylamino;

each of R², R³, R⁴, R⁵, R⁶, R⁷, R⁸, R⁹, and R¹⁰ is independently selected from the group consisting of hydrogen, alkyl, alkenyl, alkynyl, aryl, carbonyl, imine, iminium, cyano, halogen, sulfoxide, sulfonyl (R(O)₂S—), sulfonate (R(O)₂S—O—), sulfonamide, diazo, azido, nitro, nitroso, hydroxy, alkoxy, alkenoxy, aryloxy, carboxyl (RCOO—), siloxy, phosphonate ((RO)(R′O)(O)P—), phosphate ((RO)(R′O)(O)P—O—), amino, alkylamino, arylamino, dialkylamino, diarylamino, trialkyl ammonium, triaryl ammonium, trialkyl phosphonium, and triaryl phosphonium; and at least one of the R², R³, R⁴, R⁵, R⁶, R⁷, R⁸, R⁹, and R¹⁰ groups is a leaving group, which is replaced by the incoming sulfur nucleophile, selected from group consisting of hydrogen, halogen, cyano, thiolate (RS—), sulfoxide (R(O)S—), sulfonyl (R(O)₂S—), sulfonate (R(O)₂S—O—), sulfonamide, diazo, azido, nitro, nitroso, hydroxy, alkoxy, alkenoxy, aryloxy, carboxyl (RCOO—), siloxy, phosphonate ((RO)(R′O)(O)P—), phosphate ((RO)(R′O)(O)P—O—), trialkyl ammonium, triaryl ammonium, trialkyl phosphonium, and triaryl phosphonium; and

Y and Z are independently selected from the group consisting of oxygen, sulfur, selenium, amino group (—NR—), —CR(R′)—, carbonyl, thiocarbonyl, imine, alkylimine, and arylimine, in which R and R′ are independently selected from the group consisting of hydrogen, alkyl, aryl, halogen, hydroxy, alkoxy, alkenoxy, aryloxy, siloxy, nitro, nitroso, amino, alkylamino, arylamino, dialkylamino, diarylamino, and cyano.

In certain instances, any combination of two of R¹, R¹′, R², R³, R⁴, R₅, R⁶, R⁷, R⁸, R⁹, and R¹⁰ can be joined together to form a cyclic structure.

In other instances, any combination of three of R¹, R¹′, R², R³, R⁴, R₅, R⁶, R⁷, R⁸, R⁹, and R¹⁰ can be joined together to form a bicyclic structure.

Any compound described above, if not charge-neutral, should also contain one or more counter anions to form a charge-neutral entity. The number of net negative charges of the anion(s) matches that of the net positive charges of the pnictogen-containing heterocycle described above.

The compounds described above are useful in modifying a substrate containing a chalcogen functional groups such as OH, SH, SeH, and TeH. The modification reaction can be depicted in Schemes I-V below.

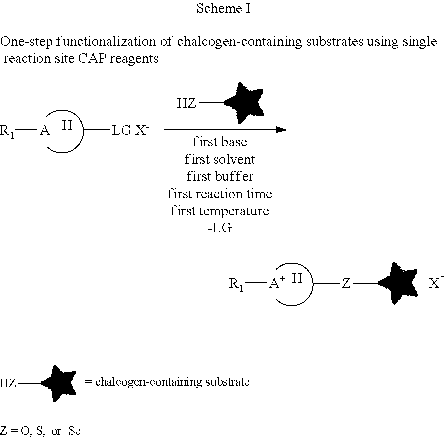

Referring to Scheme I above, R₁, A⁺,

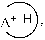

LG, and X⁻ are defined for Formula (I) above. The reaction is performed for a first reaction time (e.g., 1 minute to 48 hours, preferably 2 minutes to 12 hours, and more preferably 5 minutes to 6 hours) at a first temperature (e.g., −80° C. to 150° C., −20° C. to 100° C., and 10° C. to 40° C.), optionally in the presence of a first base, a first solvent, a first buffer, or any combination thereof.

The first base, if needed for the chemical transformation, can be ammonium hydroxide, alkylamines, dialkylamines, trialkylamines, pyridine derivatives, imidazole derivatives, guanidine derivatives, 1,8-bis(dimethylamino)naphthalene, phosphazenes, tetraalkylammonium hydroxides, trialkylarylammonium hydroxides, tetraalkylphosphonium hydroxides, tetraarylphosphonium hydroxides, alkali metal hydroxides, alkali metal alkoxides, alkali metal carbonates, alkali metal bicarbonates, alkali metal phosphates, alkaline earth metal hydroxides, alkaline earth metal alkoxides, alkaline earth metal carbonates, alkaline earth metal bicarbonates, alkaline earth metal phosphates, alkyllithium reagents, aryllithium reagents, alkali metal amides, alkali metal dialkylamides, alkali metal hexamethyldisilazides, or an acceptable salt, solvate, hydrate, polymorph, co-crystal, tautomer, or a mixture thereof;

Examples of the first solvent, if needed, include water, dimethylformamide (DMF), N-methylformamide, CH₃CN, t-butanol, (CH₃)₂CHOH, CH₃CH₂OH, CH₃OH, CF₃CH₂OH, DMSO, diethyl ether, dibutyl ether, methyl t-butyl ether, tetrahydrofuran (THF), 1,4-dioxane, dimethoxyethane, diglyme, ethylene glycol, glycerol, carbon tetrachloride, chloroform, dichloromethane, dichloroethane, acetone, 2-butanone, toluene, xylenes, fluorobenzene, chlorobenzene, α,α,α-trifluorotoluene, hexafluorobenzene, tris(2-carboxyethyl)phosphine (TCEP), and a mixture thereof.

The first buffer, if needed, typically contains Na₃PO₄, piperazine-N,N′-bis(2-ethanesulfonic acid) (PIPES)(Fisher Scientific® Company, Pittsburgh, PA), 3-(N-morpholino)propanesulfonic acid (MOPS), 4-(2-hydroxyethyl)-1-piperazineethanesulfonic acid (HEPES), 2-(N-morpholino)ethanesulfonic acid (MES), tris(hydroxymethyl)aminomethane (TRIS), tris(2-carboxyethyl)phosphine (TCEP), or a mixture thereof.

A subset of the above-described compounds each contain two, three, or more leaving groups. They can react with two functional groups as shown in Schemes II, III, and IV below.

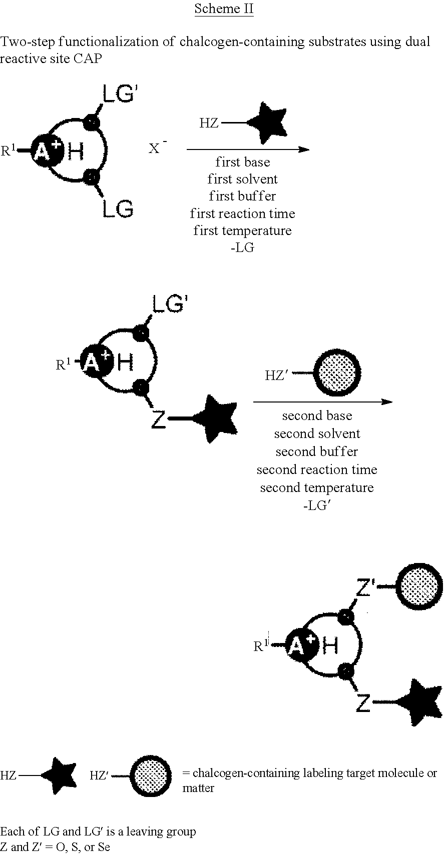

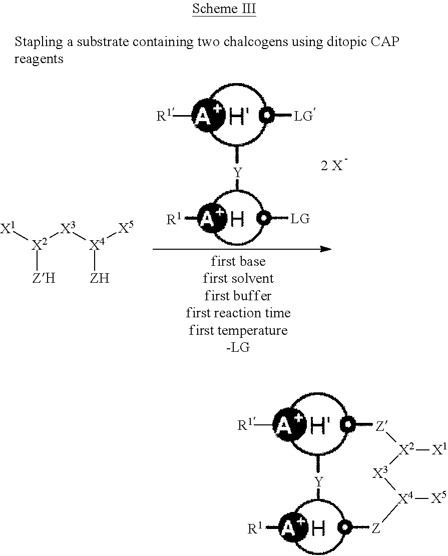

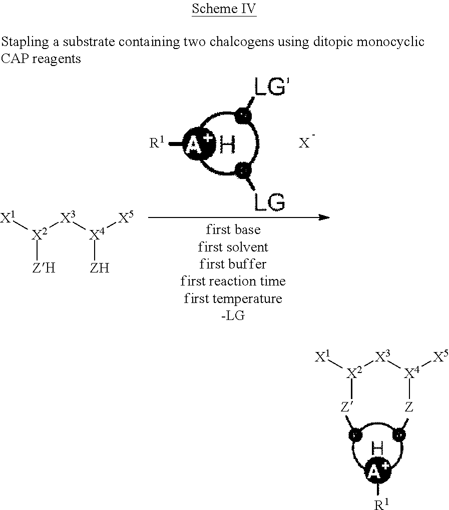

In Schemes II, III, and IV above, each of the pnictogen-containing heterocyclic compound is used to conjugate two functional groups, either in a single substrate or two separate substrates. Each substrate contains hydroxyl, thiol, or selenol, which is then covalently bond to the pnictogen-containing heterocyclic compound.

In the schemes above, LG and LG′ each are leaving groups as described above; X⁻ is a counter anion; H-Z-★ is a chalcogen-containing substrate, in which Z is oxygen, sulfur, or selenium; H-Z′-o is a chalcogen-containing substrate, wherein Z′ is oxygen, sulfur, or selenium; X¹, X², X³, X⁴, and X⁵ are each independently selected from the group consisting of a natural amino acid, an unnatural amino acid, a peptide, an oligopeptide, a polypeptide, a protein, hydrogen, an alkyl group, an alkenyl group, an alkynyl group, an aryl group, an heteroaryl group, —CR(R′)—, carbonyl, ether, thioether, sulfoxide, sulfone, phosphate, imine, alkylimine, and arylimine; wherein R and R′ are independently selected from the group consisting of hydrogen, alkyl, aryl, halogen, hydroxy, alkoxy, aryloxy, siloxy, amino, alkylamino, arylamino, dialkylamino, and diarylamino; and Y is a linker and selected from the group consisting of a natural amino acid, an unnatural amino acid, a peptide, an oligopeptide, a polypeptide, a protein, (CH₂)ₙ, an alkyl group, an alkyl group, an alkenyl group, an alkynyl group, an aryl group, an heteroaryl group, —CR(R′)—, carbonyl, ether, thioether, sulfoxide, sulfone, phosphate, imine, alkylimine, and arylimine; wherein R and R′ are independently selected from the group consisting of hydrogen, alkyl, aryl, halogen, hydroxy, alkoxy, aryloxy, siloxy, amino, alkylamino, arylamino, dialkylamino, and diarylamino, n being 0 to 50.

In Scheme II above, the CAP compound is reacted with a first substrate, followed by a reaction with a second substrate. This two-step reaction sequence leads to a covalent conjugate of two chalcogen-containing substrates.

The first base, the first solvent, the first buffer, the first temperature, and the first reaction time are as defined above.

The second reaction is carried out at a second temperature for a second reaction time, optionally in the presence of a second base, a second solvent, a second buffer, or any combinations thereof. The second base, the second solvent, the second buffer, the second temperature, and the second reaction time can be selected from the groups shown above for their counterparts in the first reaction.

In Schemes III and IV above, the substrate can a biomolecule, including peptides, proteins, modified DNA, modified RNA, synthetic polymers, and nanoparticles. The number of atoms between the ZH and Z′H ranges from 1 to 50000.

In certain embodiments, the pnictogen-containing heterocyclic compound of this invention is used to functionalize a substrate having two or more hydroxyl, thiol, and selenol groups in a sequence-dependent regioselective manner as shown in Scheme V below.

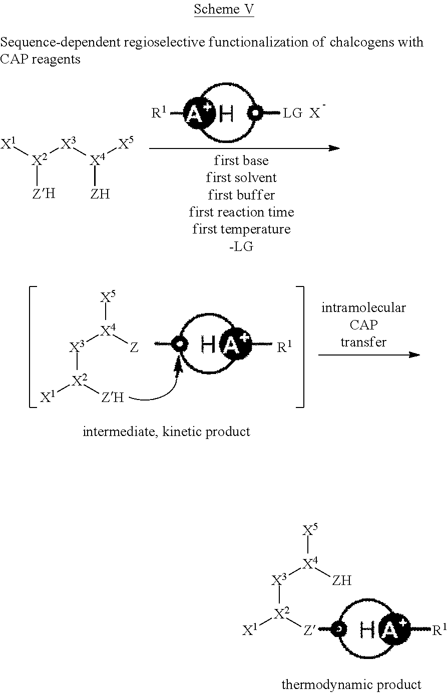

Each variable in Scheme V is as defined above. In the first step, the CAP compound reacts with the most nucleophilic hydroxyl, thiol, or selenol group (i.e., ZH). The resultant kinetic product is an intermediate, which undergoes intramolecular CAP transfer to the adjacent chalcogen (i.e., Z′H) to form a product with a higher thermodynamic stability. The sequence-dependent regioselective functionalization relies on (1) the faster reaction of the chalcogen with higher reactivity toward the CAP compound and (2) the intramolecular transfer of the CAP moiety to a spatially close nucleophilic chalcogen.

The substrate can be a biomolecule, including peptides, proteins, modified DNA, modified RNA, synthetic polymers, and nanoparticles. The number of atoms between the ZH and Z′H ranges from 1 to 50000.

Following the reaction schemes shown above, the CAP compounds of this invention are suitable to modifying substrates such as biomolecules, particularly proteins and peptides. The modified biomolecules are useful in broad applications, including the production of protein-drug conjugates for targeting specific tissues, the synthesis of stapled peptides for improved metabolic stability and cell permeability, mass spectrometry-based protein profiling studies, the incorporation of functional tags, such as fluorescent labels, into biomacromolecules for detection and imaging studies, and the development of covalent drugs by formation protein-small molecule complexes. In addition, these reactions are used in biological studies, in which protein or peptide functionality needs to be reversibly or irreversibly modified by introduction or masking of particular functional groups.

Some applications include antibody-CAP-drug conjugate and protein-CAP-drug conjugate, in which a CAP compound having two leaving groups is utilized. The antibody or protein covalently bonds the CAP compound through the substitution of the first leaving group in the CAP compound with the thiol functional group in the antibody or protein. A drug molecule is also covalently attached to the CAP compound through the substitution of the second leaving group in the CAP compound with the hydroxyl, thiol, or selenol functional group in the drug molecule.

Exemplary therapeutic protein-CAP-drug and antibody-CAP-drug conjugates are illustrated in Scheme VI below.

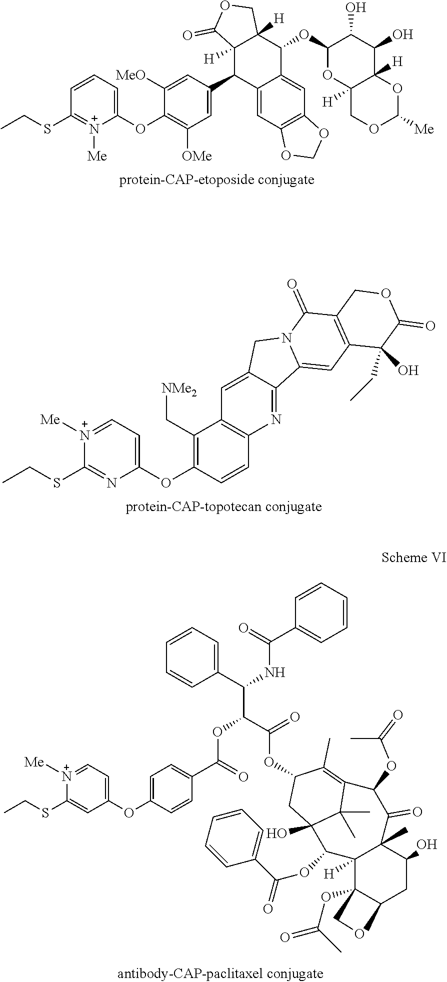 [SMILES: CCSc1cccc(Oc2c(OC)cc([C@@H]3c4cc5c(cc4[C@@H](O[C@@H]4O[C@@H]6CO[C@@H](C)O[C@H]6[C@H](O)[C@H]4O)[C@H]4COC(=O)[C@H]34)OCO5)cc2OC)[n+]1C ; CCSc1nc(Oc2ccc3nc4c(cc3c2CN(C)C)Cn2c-4cc3c(c2=O)COC(=O)[C@]3(O)CC)cc[n+]1C ; CCSc1cc(Oc2ccc(C(=O)O[C@@H](C(=O)O[C@H]3C[C@@]4(O)[C@@H](OC(=O)c5ccccc5)[C@@H]5[C@@](CO)(OC(C)=O)[C@H](C)C[C@H](O)[C@@]5(C)C(=O)[C@H](OC(C)=O)C(=C3C)C4(C)C)[C@@H](NC(=O)c3ccccc3)c3ccccc3)cc2)cc[n+]1C]

In Scheme VI above, etoposide, topotecan, and paclitaxel are all anti-cancer agents. A CAP compound is a pnictogen-containing heterocyclic compound of this invention. All proteins and antibodies can be modified by a CAP compound. Specific suitable proteins and antibodies are those containing a thiol functional group.

The compounds and conjugates described above can be prepared by synthetic methods well known in the art. See, e.g., R. Larock, Comprehensive Organic Transformations (3^{rd} Ed., John Wiley and Sons 2018); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2^{nd} ed., John Wiley and Sons 2009) and subsequent editions thereof.

The compounds thus prepared can be purified following conventional methods such as crystallization, distillation/vacuum distillation, flash chromatography over silica, and preparative liquid chromatography.

Without further elaboration, it is believed that one skilled in the art can, based on the above description, utilize the present invention to its fullest extent. The following examples are to be construed as merely illustrative and not limitative of the remainder of the disclosure in any way whatsoever.

All publications cited herein are hereby incorporated by reference in their entirety.

Set forth below are examples illustrating preparation and evaluation of compounds of this invention.

The abbreviations as used herein are provided in Table 2 below with their definitions, which are used conventionally in the art.

TABLE 2

| Abbr. | Name | Abbr. | Name |
|---|---|---|---|
| BAPNA | Nα-benzoyl-DL-arginine 4- | BSA | bovine serum albumin |
| | nitroanilide hydrochloride | | |
| CHCA | α-cyano-4- | Cys | cysteine |
| | hydroxycinnamic acid | | |
| DMF | dimethylformamide | DMSO | Dimethyl sulfoxide |
| EDTA | ethylenediaminetetraacetic | ESI-HRMS | High resolution electrospray |
| | acid | | ionization mass spectrometry |
| Et | ethyl | Et₂O | ethyl ether |
| GFP | Green Fluorescent Protein | HATU | 1-[bis(dimethylamino)- |
| | | | methylene]-1H-1,2,3-tri- |
| | | | azolo[4,5-b] pyridinium 3-oxide |
| | | | hexafluorophosphate |
| HPLC | High-Performance Liquid | MALDI-MS | matrix-assisted laser |
| | Chromatography | | desorption/ionization mass |
| | | | spectrometry |
| LCMS | Liquid Chromatography- | HD | high resolution |
| | Mass Spectrometry | | |
| Me | methyl | MeCN | acetonitrile |
| MS | mass spectrometry | NMR | nuclear magnetic resonance |
| Ph | phenyl | PIPES | piperazine-N,N′-bis(2- |
| | | | ethanesulfonic acid) |
| TAMRA | carboxytetramethyl- | TCEP | tris(2-carboxyethyl)phosphine |
| | rhodamine | | |
| THF | tetrahydrofuran | TLC | thin layer chromatography |

### EXAMPLES

Reagents were purchased from commercial sources and used as received unless otherwise indicated. Anhydrous solvents were purchased from commercial sources and stored in a dry box. Reaction mixtures were monitored by thin-layer chromatography (TLC) on pre-coated, aluminum-backed silica gel 60 F254 plates. Column chromatography was performed on silica gel 60 (230-400 mesh). NMR spectra were recorded on a Bruker® Avance III HD NMR spectrometer. ¹H and ¹³C chemical shifts are reported in ppm relative to SiMe₄ (δ=0.00 ppm). ¹H and ¹³C NMR spectra were referenced internally to residual solvent peaks. ¹⁹F spectra were referenced externally to CFCl₃ (0.0 ppm). Low-resolution electrospray mass spectra were acquired on a Shimadzu® LCMS-2020 spectrometer. High-resolution mass spectra were acquired on AB SCIEX® TripleTOF 4600 system at the RI-INBRE Centralized Research Core Facility. Compounds were purified using Agilent® 1200 Series HPLC systems fitted with multi-wavelength detectors and automated fraction collectors using a C18 reverse stationary phase (Zorbax®-SB C18 columns: preparative, 7 μm, 21.2×250 mm; semi-preparative, 5 μm 9.4×250 mm; and analytical, 5 μm, 4.6×250 mm; all commercially available from Agilent, Santa Clara, CA) and a mobile phase composed of two solvents (A: H₂O+0.1% (v/v) CF₃CO₂H; B: CH₃CN+0.1% (v/v) CF₃CO₂H) with a solvent gradient selected from those shown in Table 3 below. UV-visible spectra were recorded on a Varian® Cary 60 Bio UV-visible spectrophotometer. Quartz cuvettes with 1 cm path lengths were used for all spectroscopic measurements. Milli-Q® purified water with resistivity of at least 18 MΩ·cm⁻¹ was used to prepare all buffers. Aqueous PIPES buffers consisted of 50 mM PIPES, 100 mM KCl. Unless otherwise indicated, the pH of the buffers was adjusted to 7.

TABLE 3
HPLC Solvent Gradients

| | Time | | | Time | |
|---|---|---|---|---|---|
| Gradient | (min) | % Solvent B | Gradient | (min) | % Solvent B |
| 1^{a} | 0 | 10 | 2^{b} | 0 | 0 |
| | 3 | 35 | | 2 | 0 |
| | 15 | 38 | | 6 | 3 |
| | 17 | 100 | | 12 | 9 |
| | 19 | 10 | | 14 | 100 |
| | 20 | 10 | | 16 | 0 |
| | | | | 18 | 0 |
| 3^{b} | 0 | 10 | 4^{b} | 0 | 10 |
| | 3 | 20 | | 3 | 20 |
| | 11 | 28 | | 9 | 26 |
| | 13 | 100 | | 11 | 100 |
| | 15 | 10 | | 13 | 10 |
| | 16 | 10 | | 14 | 10 |
| 5^{b} | 0 | 10 | 6^{b} | 0 | 10 |
| | 3 | 25 | | 3 | 15 |
| | 13 | 35 | | 13 | 25 |
| | 15 | 100 | | 15 | 100 |
| | 17 | 10 | | 17 | 10 |
| | 18 | 10 | | 18 | 10 |
| 7^{b} | 0 | 10 | 8^{c} | 0 | 0 |
| | 5 | 10 | | 20 | 20 |
| | 9 | 25 | | 25 | 22.5 |
| | 14 | 100 | | 28 | 100 |
| | 16 | 10 | | 30 | 100 |
| | 19 | 10 | | 32 | 0 |
| | | | | 35 | 0 |
| 9^{c} | 0 | 0 | 10^{c} | 0 | 0 |
| | 10 | 15 | | 20 | 20 |
| | 30 | 75 | | 40 | 30 |
| | 32 | 100 | | 45 | 40 |
| | 33 | 100 | | 50 | 100 |
| | 35 | 0 | | 52 | 100 |
| | 36 | 0 | | 54 | 0 |
| | | | | 56 | 0 |
| 11^{d} | 0 | 10 | 12^{c} | 0 | 10 |
| | 5 | 10 | | 5 | 10 |
| | 30 | 100 | | 23 | 74 |
| | 33 | 100 | | 26 | 100 |
| | 36 | 10 | | 27 | 100 |
| | 40 | 10 | | 30 | 10 |
| | | | | 31 | 10 |
| 13^{c} | 0 | 0 | 14^{b} | 0 | 0 |
| | 3 | 0 | | 5 | 0 |
| | 6 | 24 | | 15 | 20 |
| | 13 | 28 | | 17 | 100 |
| | 16 | 100 | | 18 | 100 |
| | 17 | 100 | | 20 | 0 |
| | 20 | 0 | | 22 | 0 |
| | 21 | 0 | | | |
| 15^{b} | 0 | 10 | 16^{b} | 0 | 10 |
| | 5 | 28 | | 5 | 28 |
| | 19 | 69 | | 19 | 31 |
| | 21 | 100 | | 21 | 100 |
| | 22 | 100 | | 22 | 100 |
| | 25 | 10 | | 25 | 10 |
| | 30 | 10 | | 30 | 10 |
| 17^{b} | 0 | 0 | | | |
| | 5 | 5 | | | |
| | 30 | 30 | | | |
| | 33 | 33 | | | |
| | 36 | 36 | | | |
| | 40 | 40 | | | |

^{a}Flow rate was 15 mL/min.
^{b}Flow rate was 3 mL/min.
^{c}Flow rate was 4 mL/min.
^{d}Flow rate was 1 mL/min.

### 1-Iodo-9-(2-fluoro-4-pyridyl)-3,6,9-trioxanonane

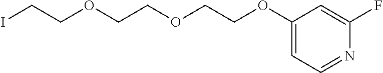 [SMILES: Fc1cc(OCCOCCOCCI)ccn1]

In a 20 mL scintillation vial, triglycol diiodide (2.22 g, 1.09 mL, 6 mmol), 2-fluoro-4-hydroxypyridine (339 mg, 3 mmol), and K₂CO₃ (415 mg, 3 mmol) were mixed in DMF (9 mL). The reaction was heated at 60° C. for 3 h, during which time the solid K₂CO₃ dissolved. The yellow reaction mixture was cooled to rt. DMF was removed by air flow. The crude product was purified by silica gel column chromatography (ethyl acetate:hexanes=1:2 to 1:1). A slightly yellow liquid was obtained (742 mg, 70% yield).

¹H NMR (400 MHz, CDCl₃) δ 8.00 (d, J=5.9 Hz, 1H), 6.73 (ddd, J=5.9, 2.1, 1.2 Hz, 1H), 6.40 (d, J=2.1 Hz, 1H), 4.21-4.16 (m, 2H), 3.91-3.86 (m, 2H), 3.74 (t, J=6.8 Hz, 2H), 3.73-3.69 (m, 2H), 3.69-3.65 (m, 2H), 3.25 (t, J=6.8 Hz, 2H). ¹⁹F NMR (377 MHz, CDCl₃) δ −66.70. ¹³C¹{H} NMR (101 MHz, CDCl₃) δ 168.80 (d, J=11.7 Hz), 165.34 (d, J=235.2 Hz), 148.15 (d, J=18.6 Hz), 109.57 (d, J=4.2 Hz), 94.83 (d, J=41.8 Hz), 72.08, 71.00, 70.35, 69.37, 68.11, 2.98. MS (ESI+): m/z calculated for [M+H]⁺ 356.0, found 356.1. MS (ESI+): m/z calculated for [M+H]⁺ 356.0, found 356.1. ESI-HRMS(+) m/z calculated for C₁₁H₁₆FINO₃⁺[M+H]⁺ 356.0154, found 356.0141.

### 1-Azido-9-(2-fluoro-4-pyridyl)-3,6,9-trioxanonane

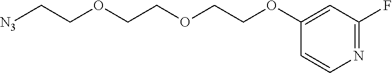 [SMILES: [N-]=[N+]=NCCOCCOCCOc1ccnc(F)c1]

In a 10 mL Schlenk flask, tetra-n-butylammonium azide (63 mg, 0.22 mmol) was added to a solution of 2-fluoro-4-(2-(2-(2-iodoethoxy)ethoxy)ethoxy)pyridine (71 mg, 0.2 mmol) in anhydrous THF (1 mL). The reaction was stirred at rt for 12 h. The solvent was removed under vacuum. The crude product was purified by silica gel column chromatography (ethyl acetate:hexanes=1:1 to 2:1). A colorless liquid was obtained (53 mg, 98% yield).

¹H NMR (400 MHz, CDCl₃) δ 8.01 (d, J=5.9 Hz, 1H), 6.73 (ddd, J=5.9, 2.1, 1.1 Hz, 1H), 6.41 (d, J=2.1 Hz, 1H), 4.21-4.17 (m, 2H), 3.92-3.86 (m, 2H), 3.74-3.71 (m, 2H), 3.69-3.65 (m, 4H), 3.38 (t, J=5.0 Hz, 2H). ¹³C¹{H} NMR (101 MHz, CDCl₃) δ 168.84 (d, J=11.7 Hz), 165.37 (d, J=235.2 Hz), 148.16 (d, J=18.5 Hz), 109.57 (d, J=3.9 Hz), 94.84 (d, J=41.8 Hz), 71.07 (s), 70.86 (s), 70.28 (s), 69.39 (s), 68.11 (s), 50.78 (s). ¹⁹F NMR (471 MHz, CDCl₃) δ −66.7 (s). MS (ESI+): m/z calculated for [M+H]⁺ 271.1, found 271.2. ESI-HRMS(+) m/z calculated for C₁₁H₁₆FN₄O₃⁺[M+H]⁺ 271.1201, found 271.1189.

### 1-(2-Fluoro-4-pyridyl)-10-propargyl-1,4,7,10-tetraoxadecane

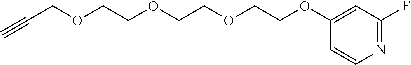 [SMILES: C#CCOCCOCCOCCOc1ccnc(F)c1]

In a 50 mL pressure tube, 3-(2-(2-(2-iodoethoxy)ethoxy)-ethoxy)prop-1-yne (149 mg, 0.5 mMol), 2-fluoro-4-hydroxypyridine (62 mg, 0.55 mmol), and K₂CO₃ (84 mg, 0.61 mmol) were mixed in anhydrous acetone (5 mL). The reaction was heated in an oil bath (80° C.) for 24 h. The solvent was removed under vacuum. The crude product was purified by silica gel column chromatography (ethyl acetate:hexanes=1:1 to 2:1). A colorless liquid was obtained (113 mg, 80% yield).

¹H NMR (400 MHz, CDCl₃) δ 8.01 (d, J=5.9 Hz, 1H), 6.73 (ddd, J=5.9, 2.0, 1.1 Hz, 1H), 6.41 (d, J=2.1 Hz, 1H), 4.21-4.14 (m, 4H), 3.90-3.85 (m, 2H), 3.75-3.64 (m, 8H), 2.42 (t, J=2.4 Hz, 1H). ¹³C¹{H} NMR (101 MHz, CDCl₃) δ 168.86 (d, J=11.6 Hz), 165.37 (d, J=235.1 Hz), 148.15 (d, J=18.6 Hz), 109.60 (d, J=3.9 Hz), 94.85 (d, J=41.8 Hz), 79.72, 74.69, 71.05, 70.79, 70.62, 69.31, 69.22, 68.14, 58.55. ¹⁹F NMR (377 MHz, CDCl₃) δ −66.8 (s). MS (ESI+): m/z calculated for [M+H]⁺ 284.1, found 284.2. MS (ESI+): m/z calculated for [M+H]⁺ 284.1, found 284.2. ESI-HRMS(+) m/z calculated for C₁₄H₁₉FNO₄+[M+H]⁺ 284.1293, found 284.1303.

### N¹-Methyl-N²-Methyl-N²-4-hydroxybenzoyl-1,2-ethylenediamine

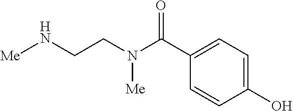 [SMILES: CNCCN(C)C(=O)c1ccc(O)cc1]

A mixture of 4-acetoxybenzoic acid (77 mg, 0.427 mmol) and thionyl chloride (286 μL, 3.84 mmol) was heated at 85° C. in a OH, pressure tube for 1 h. The mixture was cooled to room temperature. Excess thionyl chloride was removed under vacuum to afford crude 4-(chlorocarbonyl)phenyl acetate as a colorless oil. The crude 4-(chlorocarbonyl)phenyl acetate was dissolved in anhydrous CH₂Cl₂ (2 mL). To this mixture, t-butyl methyl(2-ethyl)carbamate (755 μL, 3.84 mmol) was added. The reaction was stirred at room temperature for 12 h before the addition of water (20 mL). The mixture was then extracted with CH₂Cl₂ (15 mL×3). The combined organic phase was dried over Na₂SO₄. The solvent was removed under vacuum. The crude product was passed through a pad of silica gel with ethyl acetate as the eluent to afford t-butyl (2-(4-hydroxy-N-methylbenzamido)ethyl)(methyl)-carbamate as a viscous colorless oil (121 mg, 85% yield). A portion of oil (100 mg, 0.32 mmol) was mixed with CH₂Cl₂ (2.5 mL) and trifluoroacetic acid (2.5 mL). After 30 min, the solvent and trifluoroacetic acid were removed under vacuum. A slightly yellow solid was obtained (76.2 mg as a trifluoroacetic acid salt) and used in the next step without further purification.

¹H NMR (400 MHz, MeOD) δ 7.41 (d, J=8.5 Hz, 2H), 6.88-6.82 (m, 2H), 3.80 (t, J=5.7 Hz, 2H), 3.28 (t, J=5.7 Hz, 2H, partially overlapping with CD₂HOD), 3.10 (s, 3H), 2.76 (s, 3H). ¹³C¹{H} NMR (101 MHz, MeOD) δ 175.36, 160.99, 130.75, 126.82, 116.07, 48.46 (partially overlapping with CD₃OD), 46.15 (br), 38.58 (br), 34.04. MS (ESI+): m/z calculated for M⁺ 209.1, found 209.3.

### N¹-Methyl-N-5-carbonyltetramethylrhodamine-N²-methyl-N²-4-hydroxybenzoyl-1,2-ethylenediamine

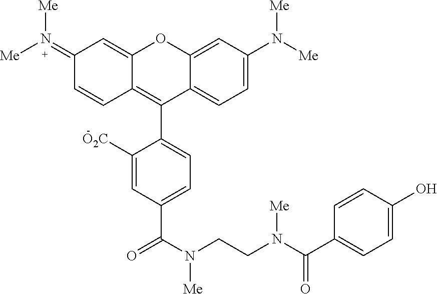

To a stirred mixture of 5-carboxytetramethylrhodamine (14.2 mg, 0.03 mmol) in DMF (100 μL), HATU (19.5 mg, 0.05 mmol) and N,N-diisopropylethylamine (50 μL, 0.3 mmol) were added at room temperature. After 5 min, a solution of N¹-Methyl-N²-Methyl-N²-4-hydroxybenzoyl-1,2-ethylenediamine (7.4 mg, 0.04 mmol) in DMF (40 μL) was added to the above mixture. The reaction was stirred at room temperature for 2 h. The reaction mixture was purified by preparative HPLC using solvent gradient 1 (Table 3, T_{R}=19.55 min) to obtain a dark purple solid (5.2 mg as a mixture with trifluoroacetic acid). Analytical HPLC using solvent gradient 11, T_{R}=18.08 min. MS (ESI+): m/z calculated for [M+H]⁺ 621.3, found 621.5. ESI-HRMS(+) m/z calculated for C₃₆H₃₇N₄O₆⁺[M+H]⁺ 621.2708, found 621.2733.

### N¹-Methyl-N-biotin-N²-methyl-N²-4-hydroxybenzoyl-1, 2-ethylenediamine

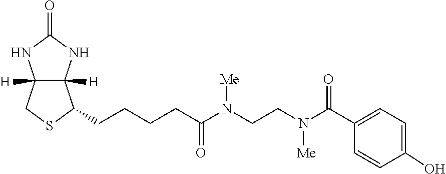 [SMILES: CN(CCN(C)C(=O)c1ccc(O)cc1)C(=O)CCCC[C@@H]1SC[C@@H]2NC(=O)N[C@@H]21]

To a stirred mixture of biotin (32 mg, 0.13 mmol) in DMF (300 μL), HATU (65.7 mg, 0.17 mmol) and N,N-diisopropyl-ethylamine (150 μL, 0.9 mmol) were added at room temperature. After 5 min, a solution of N¹-Methyl-N²-Methyl-N²-4-hydroxybenzoyl-1,2-ethylenediamine (39 mg, 0.19 mmol) in DMF (150 μL) was added to the above mixture. The reaction was stirred at room temperature for 1 h. The reaction mixture was purified by column chromatography (CH₂C₁₂: MeOH: Et₃N=8:1:0.2). The purified product was dried under vacuum to give a white solid (11.9 mg, 21% yield).

MS (ESI−): m/z calculated for [M−H]⁻ 433.2, found 433.3. ESI-HRMS(+) m/z calculated for C₂₁H₃₁N₄O₄S′ [M+H]⁺ 435.2061, found 435.2064.

### 6-Chloro-6′-(methylsulfonyl)-3,3′-bipyridine

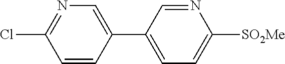 [SMILES: CS(=O)(=O)c1ccc(-c2ccc(Cl)nc2)cn1]

A mixture of THF and water (10 mL, 3:1, v/v) was added to a Schlenk flask containing 6-chloro-3-pyridinylboronic acid (47.2 mg, 0.3 mmol) and K₂CO₃ (82.9 mg, 0.6 mmol). The mixture was degassed with N₂ for 15 minutes before adding tetrakis(triphenylphosphine)palladium (57.8 mg, 0.05 mmol) and a solution of 5-iodo-2-(methylsulfonyl)pyridine (99.1 mg, 0.35 mmol) in THF (1 mL). The reaction mixture was degassed for additional two min. Under positive N₂ flow, the septum was replaced with a condenser. The stirred reaction mixture was heated to reflux for 15 hours under N₂. The reaction mixture was cooled to room temperature and filtered through a layer of diatomaceous earth (Celite®, commercially available from MilliporeSigma, Burlington, MA). THF was removed under vacuum. The resulting mixture was extracted with ethyl acetate (30 mL×4). The combined organic phase was dried over MgSO₄. The crude product was purified by column chromatography (ethyl acetate:hexanes=1:1) to afford a yellow crystalline solid (58.1 mg, 72% yield).

¹H NMR (400 MHz, CDCl₃) δ 8.99-8.86 (m, 1H), 8.66 (d, J=2.6 Hz, 1H), 8.27-8.18 (m, 1H), 8.13 (dd, J=8.1, 2.1 Hz, 1H), 7.90 (dt, J=8.3, 1.8 Hz, 1H), 7.52 (d, J=8.3 Hz, 1H), 3.28 (d, J=1.0 Hz, 3H). ¹³C{¹H} NMR (101 MHz, CDCl₃) δ 157.75, 152.71, 148.23, 148.13, 137.36, 136.43, 136.23, 130.76, 124.96, 121.45, 40.11. ESI-HRMS(+) m/z calculated for C₁₁H₁₀ClN₂O₂S⁺[M+H]⁺ 269.0147, found 269.0137.

### 2-Chloro-2′-(methylsulfonyl)-4,4′-bipyridine

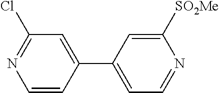 [SMILES: CS(=O)(=O)c1cc(-c2ccnc(Cl)c2)ccn1]

A mixture of THF and water (10 mL, 3:1, v/v) was added to a Schlenk flask containing 2-chloro-4-pyridinylboronic acid (39.4 mg, 0.25 mmol) and K₂CO₃ (82.9 mg, 0.6 mmol). The mixture was degassed with N₂ for 15 minutes before adding tetrakis(triphenylphosphine)palladium (57.8 mg, 0.05 mmol) and a solution of 4-iodo-2-(methylsulfonyl)pyridine (84.9 mg, 0.3 mmol) in THF (1 mL). The reaction mixture was degassed for additional two min. Under positive N₂ flow, the septum was replaced with a condenser. The stirred reaction mixture was heated to reflux for 15 hours under N₂. It was subsequently cooled to room temperature and filtered through a layer of Celite®. THF was removed under vacuum. The resulting mixture was extracted with ethyl acetate (30 mL×4). The combined organic phases were dried over MgSO₄. The crude product was purified by column chromatography (ethyl acetate:hexanes=2:1) to afford a yellow crystalline powder (35 mg, 42% yield).

¹H NMR (400 MHz, CDCl₃) δ 8.87 (d, J=5.0 Hz, 1H), 8.58 (d, J=5.2 Hz, 1H), 8.31 (d, J=1.7 Hz, 1H), 7.77 (dd, J=5.0, 1.8 Hz, 1H), 7.63 (d, J=1.6 Hz, 1H), 7.51 (dd, J=5.2, 1.6 Hz, 1H), 3.30 (s, 3H). ¹³C{¹H} NMR (101 MHz, CDCl₃) δ 159.59, 153.19, 151.26, 151.08, 147.19, 146.97, 125.07, 122.31, 120.31, 118.94, 40.10. ESI-HRMS(+) m/z calculated for C₁₁H₁₀ClN₂O₂S⁺[M+H]⁺ 269.0147, found 269.0137.

### 2-Chloro-2′-(methylsulfonyl)-3,3′-bipyridine

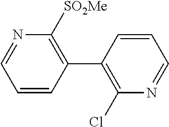 [SMILES: CS(=O)(=O)c1ncccc1-c1cccnc1Cl]

A mixture of THF and water (10 mL, 3:1, v/v) was added to a Schlenk flask containing 2-chloro-3-pyridinylboronic acid (47.2 mg, 0.3 mmol) and K₂CO₃ (82.9 mg, 0.6 mmol). The mixture was degassed with N₂ for 15 minutes before adding tetrakis(triphenylphosphine)palladium (57.8 mg, 0.05 mmol) and a solution of 3-iodo-2-(methylsulfonyl)pyridine (99.1 mg, 0.35 mmol) in THF (1 mL). Under positive N₂ flow, the septum was replaced with a condenser. The stirred reaction mixture was heated to reflux for 15 hours under N₂. The reaction mixture was cooled to room temperature and filtered through a layer of Celite©. THF was removed under vacuum. The resulting mixture was extracted with ethyl acetate (30 mL×4). The combined organic phase was dried over MgSO₄. The crude product was purified by column chromatography (ethyl acetate:hexanes=1.5:1 to 2:1) to afford a yellow crystalline solid (46.1 mg, 57% yield).

¹H NMR (400 MHz, CDCl₃) δ 8.74 (dd, J=4.6, 1.6 Hz, 1H), 8.49 (dd, J=4.8, 1.9 Hz, 1H), 7.79 (ddd, J=11.6, 7.7, 1.8 Hz, 2H), 7.64 (dd, J=7.8, 4.7 Hz, 1H), 7.36 (dd, J=7.6, 4.8 Hz, 1H), 3.31 (s, 3H). ¹³C{¹H} NMR (101 MHz, CDCl₃) δ 155.86, 149.96, 149.55, 148.90, 141.22, 140.14, 131.73, 131.01, 126.63, 121.92, 40.34. ESI-MS(+) m/z calculated for [M+H]⁺ 269.0, found 269.1. ESI-HRMS(+) m/z calculated for C₁₁H₁₀ClN₂O₂S⁺[M+H]⁺ 269.0147, found 269.0135.

### 5-Iodo-2-(methylsulfonyl)pyridine

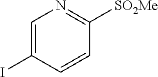 [SMILES: CS(=O)(=O)c1ccc(I)cn1]

An aliquot of H₂O₂(50 mM, 30 wt % in water, 5.11 mL) was added dropwise to a stirred mixture of 5-iodo-2-(methylthio)pyridine (251 mg, 1 mmol), sodium tungstate dihydrate (132 mg, 0.4 mmol), water (4 mL), and ethyl acetate (6 mL) at room temperature. The progress of the reaction was monitored by TLC. Upon the completion, the reaction mixture was cooled to 0° C. and quenched with solid Na₂SO₃ (0.5 g). The aqueous layer was separated from the organic phase and extracted with ethyl acetate (30 mL×3). The combined organic phase was dried over MgSO₄. The solvents were removed under vacuum. The crude product was purified by column chromatography (ethyl acetate:hexanes=1:1). A colorless crystals were obtained (235 mg, 83%).

¹H NMR (400 MHz, CDCl₃) δ 8.93 (d, J=2.0 Hz, 1H), 8.31 (dd, J=8.2, 2.0 Hz, 1H), 7.85 (dd, J=8.1, 0.8 Hz, 1H), 3.21 (s, 3H). ¹³C{¹H} NMR (101 MHz, CDCl₃) δ 157.08, 156.26, 146.75, 122.65, 98.57, 40.19. ESI-HRMS(+) m/z calculated for C₆H₇INO₂S⁺[M+H]⁺ 283.9237, found 283.9217.

### 4-Iodo-2-(methylsulfonyl)pyridine

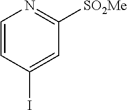 [SMILES: CS(=O)(=O)c1cc(I)ccn1]

An aliquot of H₂O₂(36.5 mM, 30 wt % in water, 3.73 mL) was added dropwise to a stirred mixture of 4-iodo-2-(methylthio)pyridine (204 mg, 0.81 mmol), sodium tungstate dihydrate (17.2 mg, 0.052 mmol), water (3 mL), and ethyl acetate (6 mL) at room temperature. The progress of the reaction was monitored by TLC. Upon the completion, the reaction mixture was cooled to 0° C. and quenched with solid Na₂SO₃ (0.5 g). The aqueous layer was separated from the organic phase and extracted with ethyl acetate (30 mL×3). The combined organic phase was dried over MgSO₄. The solvents were removed under vacuum. The crude product was purified by column chromatography (ethyl acetate:hexanes=1:1). Colorless crystals were obtained (187 mg, 82% yield).

¹H NMR (400 MHz, CDCl₃) δ 8.45 (d, J=1.5 Hz, 1H), 8.37 (d, J=5.0 Hz, 1H), 7.95 (dt, J=5.1, 1.3 Hz, 1H), 3.23 (s, 3H). ¹³C{¹H} NMR (101 MHz, CDCl₃) δ 158.38, 150.27, 136.86, 130.41, 107.18, 40.13. ESI-MS(+) m/z calculated for [M+H]⁺ 283.9, found 284.0.

ESI-HRMS(+) m/z calculated for C₆H₇INO₂S⁺[M+H]⁺ 283.9237, found 283.9217.

### 3-Iodo-2-(methylsulfonyl)pyridine

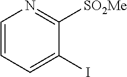 [SMILES: CS(=O)(=O)c1ncccc1I]

An aliquot of H₂O₂(70 mM, 30 wt % in water, 7.15 mL) was added dropwise to a stirred mixture of 4-iodo-2-(methylthio)pyridine (251 mg, 1 mmol), sodium tungstate dihydrate (33 mg, 0.1 mmol), water (4 mL), and ethyl acetate (6 mL) at room temperature. The progress of the reaction was monitored by TLC. Upon the completion, the reaction mixture was cooled to 0° C. and quenched with solid Na₂SO₃ (0.5 g). The aqueous layer was separated from the organic phase and extracted with ethyl acetate (30 mL×3). The combined organic phase was dried over MgSO₄. The solvents were removed under vacuum. The crude product was purified by column chromatography (ethyl acetate:hexanes=1:1). Colorless crystals were obtained (209 mg, 74% yield).

¹H NMR (400 MHz, CDCl₃) δ 8.57 (dd, J=4.7, 1.4 Hz, 1H), 8.40 (dt, J=8.0, 1.2 Hz, 1H), 7.25-7.18 (m, 1H), 3.44 (d, J=0.9 Hz, 3H). ¹³C¹{H} NMR (101 MHz, CDCl₃) δ 158.19, 150.71, 147.07, 127.42, 85.05, 39.54. ESI-HRMS(+) m/z calculated for C₆H₇INO₂S⁺[M+H]⁺ 283.9237, found 283.9229.

### 5-Iodo-2-(methylthio)pyridine

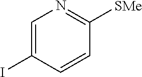

A portion of 2-fluoro-5-iodopyridine (446 mg, 2 mmol) and sodium thiomethoxide (147 mg, 2.1 mmol) were mixed in anhydrous THF (2 mL) in a vial in the glovebox. The reaction stirred at room temperature for 15 h. The solvent was removed under vacuum. The crude product was purified by column chromatography (hexanes:ethyl acetate=97:3) to give a white solid (495 mg, 99%).

¹H NMR (400 MHz, CDCl₃) δ 8.62 (d, J=1.9 Hz, 1H), 7.72 (dd, J=8.5, 2.2 Hz, 1H), 6.99 (d, J=8.5 Hz, 1H), 2.53 (s, 3H). ¹³C{¹H} NMR (101 MHz, CDCl₃) δ 159.36, 155.17, 143.58, 123.22, 86.92, 13.28. ESI-HRMS(+) m/z calculated for C₆H₇INS⁺[M+H]⁺ 251.9338, found 251.9333.

### 4-Iodo-2-(methylthio)pyridine

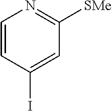

A portion of 2-fluoro-4-iodopyridine (446 mg, 2 mmol) and sodium thiomethoxide (147 mg, 2.1 mmol) were mixed in anhydrous THF (2 mL) in a vial in the glovebox. The reaction stirred at room temperature for 15 h. The solvent was removed under vacuum. The crude product was purified by column chromatography (hexanes:ethyl acetate=95: 5) to give a white solid (435 mg, 87% yield).

¹H NMR (400 MHz, CDCl₃) δ 8.07 (d, J=5.3 Hz, 1H), 7.57 (d, J=1.5 Hz, 1H), 7.33 (dd, J=5.3, 1.5 Hz, 1H), 2.54 (s, 3H). ¹³C{¹H} NMR (101 MHz, CDCl₃) δ 161.67, 149.55, 130.15, 128.30, 105.08, 13.43, 0.14. ESI-HRMS(+) m/z calculated for C₆H₇INS⁺[M+H]⁺ 251.9338, found 251.9325.

### 2-Fluoro-6-(methylthio)pyridine

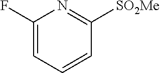 [SMILES: CS(=O)(=O)c1cccc(F)n1]

An aliquot of H₂O₂(12 mM, 30 wt % in water, 1.23 mL) was added dropwise to a stirred mixture of 2-fluoro-6-(methylthio)pyridine (572 mg, 4 mmol), sodium tungstate dihydrate (132 mg, 0.4 mmol), water (1 mL), and ethyl acetate (8 mL) at room temperature. The progress of the reaction was monitored by TLC. After three hours, the reaction mixture was cooled to 0° C. and quenched with solid Na₂SO₃ (0.6 g). The aqueous layer was separated from the organic phase and extracted with ethyl acetate (30 mL×3). The combined organic phase was dried over Na₂SO₄. The solvents were removed under vacuum. The crude product was purified by column chromatography (ethyl acetate:hexanes=1:1). A slightly yellow oil was obtained (625 mg, 89%).

¹H NMR (400 MHz, CDCl₃) δ 8.09 (dd, J=15.5, 7.4 Hz, 1H), 8.00 (ddd, J=7.4, 1.9, 0.6 Hz, 1H), 7.23 (ddd, J=8.2, 2.5, 0.5 Hz, 1H), 3.23 (s, 3H). ¹³C{¹H} NMR (101 MHz, CDCl₃) δ 162.79 (d, J=250.0 Hz), 156.06 (d, J=11.8 Hz), 143.48 (d, J=7.9 Hz), 118.84 (d, J=4.1 Hz), 114.73 (d, J=35.4 Hz), 39.96 (s). ESI-HRMS(+) m/z calculated for C₆H₇FNO₂S⁺[M+H]⁺ 176.0177, found 176.0162.

### 1-Azido-9-(4-methylthio-2-pyridyl)-3,6,9-trioxanonane

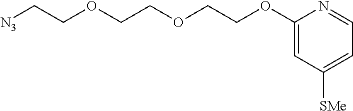 [SMILES: CSc1ccnc(OCCOCCOCCN=[N+]=[N-])c1]

A portion of sodium hydride (7.2 mg, 0.3 mmol) was added to a solution of 2-[2-(2-azidoethoxy)ethoxy]ethanol (43.8 mg, 38.6 μL, 0.25 mmol) in anhydrous 1,4-dioxane (1 mL) in a vial in the glovebox. A portion of 2-chloro-4-(methylthio)pyridine (47.9 mg, 0.3 mmol) was added to this mixture. The vial was transferred out of the glovebox and the reaction was heated at 100° C. for 1.5 h. The reaction was cooled to room temperature and quenched with MeOH (10 μL) under N₂ atmosphere. The solvent was removed under vacuum. The crude product was purified by column chromatography (ethyl acetate:hexanes=1:3 to 1:2 to 1:1). A colorless oil was obtained (51.4 mg, 69% yield).

¹H NMR (400 MHz, CDCl₃) δ 7.90 (d, J=5.6 Hz, 1H), 6.71 (dd, J=5.6, 1.6 Hz, 1H), 6.56 (d, J=1.4 Hz, 1H), 4.48-4.44 (m, 2H), 3.86-3.82 (m, 2H), 3.74-3.64 (m, 6H), 3.37 (t, J=5.1 Hz, 2H), 2.44 (s, 3H). ¹³C NMR (101 MHz, CDCl₃) δ 163.71, 153.08, 145.41, 114.55, 106.04, 70.82 (2C), 70.19, 69.91, 65.54, 50.79, 13.89. ESI-HRMS(+) m/z calculated for C₁₂H₁₉N₄O₃S⁺[M+H]⁺ 299.1173, found 299.1186.

### 1-Azido-9-(4-methylsulfonyl-2-pyridyl)-3,6,9-trioxanonane

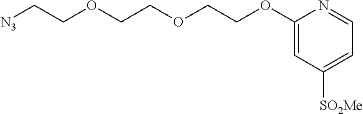 [SMILES: CS(=O)(=O)c1ccnc(OCCOCCOCCN=[N+]=[N-])c1]

An aliquot of H₂O₂(4.08 mM, 30 wt % in water, 417 μL) was added dropwise to a stirred mixture of 1-azido-9-(4-methylthio-2-pyridyl)-3,6,9-trioxanonane (50.7 mg, 0.17 mmol), sodium tungstate dihydrate (5.6 mg, 0.017 mmol), water (213 μL), and ethyl acetate (1.7 mL) at room temperature. The progress of the reaction was monitored by TLC. After 12 h, the reaction mixture was cooled to 0° C. and quenched with solid Na₂SO₃ (0.5 g). The aqueous layer was separated from the organic phase and extracted with ethyl acetate (30 mL×3). The combined organic phase was dried over Na₂SO₄. The solvents were removed under vacuum. The crude product was purified by column chromatography (ethyl acetate: hexanes=1:1). A slightly yellow oil was obtained (49.2 mg, 88%).

¹H NMR (400 MHz, CDCl₃) δ 8.37 (d, J=5.3 Hz, 1H), 7.32 (dd, J=5.3, 1.4 Hz, 1H), 7.29 (s, 1H), 4.56-4.51 (m, 2H), 3.90-3.85 (m, 2H), 3.75-3.65 (m, 6H), 3.38 (t, J=5.0 Hz, 2H), 3.05 (s, 3H). ¹³C NMR (101 MHz, CDCl₃) δ 164.53, 150.90, 148.99, 113.16, 109.71, 70.89, 70.85, 70.23, 69.59, 66.35, 50.79, 43.80. ESI-MS(+) m/z calculated for [M+H]⁺ 331.1, found 331.2. ESI-HRMS(+) m/z calculated for C₁₂H₁₉N₄O₅S⁺[M+H]⁺ 331.1071, found 331.1059.

### 1-Azido-9-(2-methylthio-4-pyridyl)-3,6,9-trioxanonane

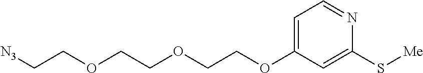 [SMILES: CSc1cc(OCCOCCOCCN=[N+]=[N-])ccn1]

Sodium thiomethoxide (30.8 mg, 0.44 mmol) was suspended in anhydrous THF (0.8 mL). A portion of 1-azido-9-(2-fluoro-4-pyridyl)-3,6,9-trioxanonane (108 mg, 0.4 mmol) in anhydrous THF (0.8 mL) was added to this mixture dropwise at 0° C. The reaction was gradually warmed to r.t. and stirred for 24 h. The solvents were evaporated and purified by column chromatography (ethyl acetate:hexanes=1:1). A colorless liquid was obtained (102 mg, 85% yield).

¹H NMR (400 MHz, CDCl₃) δ 8.24 (d, J=5.9 Hz, 1H), 6.71 (d, J=2.2 Hz, 1H), 6.58 (dd, J=5.8, 2.2 Hz, 1H), 4.17-4.13 (m, 2H), 3.87-3.84 (m, 2H), 3.74-3.64 (m, 6H), 3.37 (t, J=5.0 Hz, 2H), 2.55 (s, 3H). ¹³C NMR (101 MHz, CDCl₃) δ 165.13, 161.46, 149.93, 107.64, 106.76, 71.03, 70.84, 70.24, 69.48, 67.52, 50.77, 13.70. ESI-HRMS(+) m/z calculated for C₁₂H₁₉N₄O₃S⁺[M+H]⁺ 299.1173, found 299.1166.

### 1-Azido-9-(2-methylsulfonyl-4-pyridyl)-3,6,9-trioxanonane

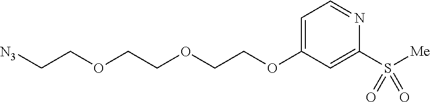 [SMILES: CS(=O)(=O)c1cc(OCCOCCOCCN=[N+]=[N-])ccn1]

An aliquot of H₂O₂(2 mM, 30 wt % in water, 204 μL) was added in two portions dropwise to a stirred mixture of 1-azido-9-(2-methylthio-4-pyridyl)-3,6,9-trioxanonane (149 mg, 0.5 mMol), sodium tungstate dihydrate (16.5 mg, 0.05 mmol), water (3 mL), and ethyl acetate (6 mL) at room temperature. The progress of the reaction was monitored by TLC. The reaction mixture was cooled to 0° C. and quenched with solid Na₂SO₃ (0.5 g). The aqueous layer was separated from the organic phase and extracted with ethyl acetate (30 mL×3). The combined organic phase was dried over Na₂SO₄. The solvents were removed under vacuum. The crude product was purified by column chromatography (ethyl acetate:hexanes=3:1 to 1:0). A slightly yellow oil was obtained (116 mg, 70%).

¹H NMR (400 MHz, CDCl₃) δ 8.50 (t, J=9.2 Hz, 1H), 7.62 (d, J=2.4 Hz, 1H), 7.04 (dd, J=5.6, 2.5 Hz, 1H), 4.30-4.25 (m, 2H), 3.93-3.88 (m, 2H), 3.70 (ddd, J=10.3, 7.3, 4.1 Hz, 6H), 3.38 (t, J=5.0 Hz, 2H), 3.21 (s, 3H). ¹³C NMR (101 MHz, CDCl₃) δ 166.69, 159.66, 151.33, 114.28, 107.71, 71.12, 70.86, 70.29, 69.36, 68.47, 50.80, 40.13. ESI-MS(+) m/z calculated for [M+Na]⁺353.1, found 353.2.

### Example 1-a: N-methyl-2-fluoropyridinium iodide (CAP1-a)

In a 50 mL pressure tube, o-fluoropyridine (583 mg, 516 μL, 6 mmol) and methyl iodide (1.7 g, 747 μL, 12 mmol) were mixed and heated at 80° C. A bright yellow solid was formed after 24 h and another portion of methyl iodide (1.7 g, 747 μL, 12 mmol) was added. The reaction was heated at 80° C. for an additional 72 h and then cooled to rt. The resultant solid was collected by suction filtration and washed with a small amount of cold MeCN (−20° C.). A slightly yellow solid was obtained (1.08 g, 75%). Crystallization was achieved by layering a solution of the solid in MeCN (about 6 mL) with dry Et₂O (about 40 mL). White needles were formed after 24 h and collected after seven days (723 mg, 50%). The brown mother liquor (about 6 mL) was layered with Et₂O (about 40 mL). White needles formed after 24 h and were collected after three days (101 mg, 7%). The overall yield was 57%. See Table 1 above for structure of CAP1-a as well as structures of CAP1-b, 1F, 1-N₃, 1-CCH, and 2-27.

¹H NMR (400 MHz, D₂O) δ 8.79-8.59 (m, 2H), 7.99-7.80 (m, 2H), 4.28 (d, J=3.8 Hz, 3H). ¹⁹F NMR (376 MHz, D₂O) δ −77.80 (s). MS (ESI+): m/z calculated for M⁺ 112.1, found 112.5. ESI-HRMS(+) m/z calculated for C₆H₇FN⁺[M]⁺112.0558, found 112.0555.

### Example 1-b: N-methyl-2-fluoropyridinium tetrafluoroborate (CAP1-b)

In a 50 mL pressure tube, o-fluoropyridine (583 mg, 516 μL, 6 mmol) and methyl iodide (1.7 g, 747 μL, 12 mmol) were mixed and heated at 80° C. A bright yellow solid formed after 24 h and another portion of methyl iodide (1.7 g, 747 μL, 12 mmol) was added. The reaction was heated at 80° C. for an additional 72 h. The reaction was cooled to rt. The solid was collected by suction filtration and washed with a small amount of cold MeCN (−20° C.). A slightly yellow solid was obtained (1.08 g, 75%). Crystallization was achieved by layering a solution of the solid in MeCN (about 6 mL) with dry Et₂O (about 40 mL). White needles formed after 24 h and were collected after seven days (723 mg, 50%). The brown mother liquor (about 6 mL) was layered with Et₂O (about 40 mL). White needles formed after 24 h and were collected after three days (101 mg, 7%). The overall yield was 57%.

¹H NMR (400 MHz, D₂O) δ 8.79-8.59 (m, 2H), 7.99-7.80 (m, 2H), 4.28 (d, J=3.8 Hz, 3H). ¹⁹F NMR (376 MHz, D₂O) δ −77.80 (s).−151.87 (s, ¹⁰BF₄⁻), −151.92 (s, ¹¹BF₄⁻).

### Example 1-F: 2,6-Difluoro-N-methylpyridinium tetrafluoroborate (CAP1-F)

In a glovebox, 2,6-difluoropyridine (1.72 g, 1.35 mL, 14.9 mmol) and trimethyloxonium tetrafluoroborate (651 mg, 4.4 mmol) were added to a vial. The mixture was stirred. A brown slurry formed after 48 h. The mixture was diluted with anhydrous MeCN (about 4 mL) and layered with anhydrous Et₂O (about 30 mL). Colorless needles formed after several days. The crystals were collected and washed with anhydrous Et₂O and dried under vacuum (871 mg, 91% yield).

¹H NMR (400 MHz, CD₃CN) δ 8.75 (tt, J=8.6, 6.8 Hz, 1H), 7.72 (d, J=8.6 Hz, 2H), 4.04 (t, J=3.1 Hz, 3H). ¹³C¹{H} NMR (101 MHz, CD₃CN) δ 158.20 (d, J=282.2 Hz), 154.90 (t, J=12.1 Hz), 111.26-110.79 (m), 35.33 (t, J=6.2 Hz). ¹⁹F NMR (377 MHz, CD₃CN) δ −79.54 (2F), −151.89 (s, ¹⁰BF₄⁻), −151.94 (s, ¹¹BF₄⁻).

ESI-HRMS(+) m/z calculated for C₆H₆F₂N⁺[M]⁺130.0463, found 130.0463.

### Example 1-N₃: 1-ethyl-4-(9-azido-1,4,7-trioxanonanyl)-2-fluoro-4-pyridinium tetrafluoroborate (CAP1-N₃)

In a glovebox, Et₃OBF₄ crystals (0.105 mmol, 20 mg) were added to 1-azido-9-(2-fluoro-4-pyridyl)-3,6,9-trioxanonane (27 mg, 0.1 mmol) in CH₂Cl₂ (400 μL). The reaction was stirred at r.t. for 24 h. The solvent was removed under vacuum. A sticky liquid was obtained (38.2 mg, 99%).

¹H NMR (400 MHz, CD₃CN) δ 8.30 (dd, J=7.3, 5.9 Hz, 1H), 7.30 (dd, J=7.3, 2.7 Hz, 1H), 7.26 (dd, J=7.1, 2.7 Hz, 1H), 4.48-4.44 (m, 2H), 4.39 (qd, J=7.3, 2.3 Hz, 2H), 3.88-3.85 (m, 2H), 3.66-3.58 (m, 6H), 3.35 (t, J=4.9 Hz, 2H), 1.49 (t, J=7.3 Hz, 3H). ¹³C¹{H} NMR (101 MHz, CD₃CN) δ 175.56 (d, J=14.0 Hz), 161.03 (d, J=272.7 Hz), 144.07 (d, J=4.7 Hz), 113.25 (d, J=1.4 Hz), 100.06 (d, J=24.7 Hz), 72.57 (s), 71.41 (s), 71.03 (s), 70.54 (s), 69.25 (s), 51.46 (s), 50.66 (d, J=4.5 Hz), 15.00 (s). ¹⁹F NMR (377 MHz, CD₃CN) δ −82.57-−82.60 (m, 1F), −151.62 (s, ¹⁰BF₄⁻), −151.67 (s, ¹¹BF₄⁻). MS (ESI+): m/z calculated for [M]⁺299.2, found 299.2. MS (ESI+): m/z calculated for [M]⁺299.2, found 299.2. ESI-HRMS(+) m/z calculated for C₁₃H₂₀FN₄O₃⁺[M]⁺299.1514, found 299.1519).

### Example 1-CCH: 1-Ethyl-4-(10-propargyl-1,4,7,10-tetraoxadecaneyl)-2-fluoro-4-pyridinium tetrafluoroborate (CAP1-CCH)

In a glovebox, Et₃OBF₄ crystals (0.105 mmol, 20 mg) were added to a solution of 1-(2-fluoro-4-pyridyl)-10-propargyl-1,4,7,10-tetraoxadecane (28.3 mg, 0.1 mmol) in CH₂Cl₂ (400 μL). The reaction was stirred at r.t. for 24 h. The solvent was removed under vacuum. A sticky liquid was obtained (39.5 mg, 99%).

¹H NMR (400 MHz, CD₃CN) δ 8.31 (dd, J=7.2, 5.9 Hz, 1H), 7.32 (dd, J=7.3, 2.7 Hz, 1H), 7.26 (dd, J=7.1, 2.7 Hz, 1H), 4.49-4.44 (m, 2H), 4.40 (qd, J=7.3, 2.3 Hz, 2H), 4.14 (d, J=2.4 Hz, 2H), 3.89-3.84 (m, 2H), 3.65-3.53 (m, 8H), 2.71 (t, J=2.4 Hz, 1H), 1.50 (t, J=7.3 Hz, 3H). ¹³C¹{H} NMR (101 MHz, CD₃CN) δ 175.55 (d, J=14.0 Hz), 161.03 (d, J=272.3 Hz) 144.12 (d, J=4.8 Hz), 113.26 (d, J=2.0 Hz), 100.10 (d, J=24.3 Hz), 80.94, 75.76, 72.58, 71.43, 71.03, 70.92, 69.93, 69.22, 58.69, 50.67 (d, J=4.8 Hz), 15.02. ¹⁹F NMR (376 MHz, CD₃CN) δ −82.54 −82.58 (m, 1F), −151.58 (s, ¹⁰BF₄⁻),−151.63 (s, ¹¹BF₄⁻). MS (ESI+): m/z calculated for M⁺ 312.4, found 312.2. MS (ESI+): m/z calculated for M⁺ 312.4, found 312.2.

ESI-HRMS(+) m/z calculated for C₁₆H₂₃FNO₄⁺[M]⁺312.1606, found 312.1602.

### Example 2: 2-Chloro-1,3-dimethyl-1H-benzimidazol-3-ium tetrafluoroborate (CAP2)

In a glovebox, 2-chloro-1-methyl-benzimidazole (666 mg, 4 mmol) and trimethyloxonium tetrafluoroborate (651 mg, 4.4 mmol) were mixed with anhydrous MeCN (4 mL). The mixture was stirred for 24 h. The mixture was filtered through a cotton plug and diluted with anhydrous MeCN (about 4 mL) and layered with anhydrous Et₂O (about 30 mL). After seven days, formed colorless needles were collected and washed with anhydrous Et₂O (953 mg, 89% yield).

¹H NMR (400 MHz, CD₃CN) δ 7.88-7.82 (m, 2H), 7.75-7.69 (m, 2H), 4.02 (s, 6H). ¹³C¹{H} NMR (101 MHz, CD₃CN) δ 141.93, 132.56, 128.36, 118.36, 113.93, 33.75. MS (ESI+): m/z calculated for M+181.1, found 181.6. ESI-HRMS(+) m/z calculated for C₉H₁₀ClN₂+[M]⁺181.0528, found 181.0528.

### Example 3: N-Methyl-4-(methylsulfonyl)pyridinium tetrafluoroborate (CAP3)

In a glovebox, 4-(methylsulfonyl)pyridine (157 mg, 1 mmol) and trimethyloxonium tetrafluoroborate (177 mg, 1.2 mmol) were mixed in anhydrous MeNO₂ (200 μL). The reaction was stirred at rt for 36 h, which led to the formation of a large amount white solid. The reaction mixture was dissolved MeCN (10 mL) and filtered through a cotton plug. Diethyl ether was diffused into the filtrate. After a week, colorless crystals formed. The crystals were collected after seven days (235 mg, 91% yield).

¹H NMR (400 MHz, CD₃CN) δ 8.95 (d, J=6.2 Hz, 2H), 8.44 (dt, J=6.4, 2.6 Hz, 2H), 4.42 (s, 3H), 3.28 (s, 3H). ¹³C{¹H} NMR (101 MHz, CD₃CN) δ 155.92 (t, J=3.3 Hz), 148.86 (t, J=9.5 Hz), 126.98, 50.17 (t, J=5.1 Hz), 43.35. ¹⁹F NMR (376 MHz, CD₃CN) δ −151.49 (s, ¹⁰BF₄), −151.55 (s, ¹¹BF₄⁻). MS (ESI+): m/z calculated for M⁺ 172.2, found 172.2. ESI-HRMS(+) m/z calculated for C₇H₁₀NO₂S⁺[M]⁺172.0427, found 172.0443.

### Example 4: N-methyl-2-(methylsulfonyl)pyridinium tetrafluoroborate (CAP4)

In a glovebox, Me₃OBF₄ (222 mg, 1.5 mmol) was mixed with the o-(methyl-sulfonyl)pyridine (157 mg, 1 mmol). A portion of anhydrous MeNO₂ (100 μL) was added. The reaction was stirred at room temperature for 36 h, during which the white solid slowly dissolved. The reaction mixture was then dissolved in anhydrous MeCN (4 mL) and filtered through a 0.2 μm PTFE syringe filter. Diethyl ether was diffused into the filtrate at −35° C. After 24 h, additional diethyl ether (30 mL) was added to the top of the mixture. Colorless crystals were collected after a week (231 mg, 89% yield).

¹H NMR (400 MHz, D₂O) δ 9.13 (d, J=6.0 Hz, 1H), 8.87 (td, J=7.9, 1.1 Hz, 1H), 8.81 (dd, J=8.0, 1.6 Hz, 1H), 8.37 (ddd, J=7.7, 6.1, 1.6 Hz, 1H), 4.74 (s, 3H), 3.64 (s, 3H). ¹³C¹{H} NMR (101 MHz, D₂O) δ 150.52, 148.30, 132.09, 130.32, 47.82, 42.95. ESI-HRMS(+) m/z calculated for C₇H₁₀NO₂S⁺[M]⁺172.0427, found 172.0442.

### Example 5: 2-Chloro-1-methyl-6-(methylsulfonyl)pyridin-1-ium tetrafluoroborate (CAP5)

A portion of 2-chloro-6-(methylsulfonyl)pyridine (96 mg, 0.5 mmol) and trimethyloxonium tetrafluoroborate (89 mg, 0.6 mmol) were mixed with anhydrous CH₃NO₂ (185 μL) in a vial in the glovebox. The reaction was stirred at room temperature for 36 hours. The reaction mixture was diluted with anhydrous CH₃CN (4 mL) and filtered through a cotton plug. The filtrate was diffused with anhydrous Et₂O. Colorless crystals formed after seven days. The crystals were washed with Et₂O and dried under vacuum (32 mg, 22%).

¹H NMR (400 MHz, CD₃CN) δ 8.79-8.62 (m, 2H), 8.42 (dd, J=7.8, 2.1 Hz, 1H), 4.61 (s, 3H), 3.52 (s, 3H). ¹³C{¹H} NMR (101 MHz, CD₃CN) δ 154.18, 153.94, 149.28, 135.48, 130.13, 45.40, 44.50.

### Example 6: 6-Chloro-1,1′-dimethyl-6′-(methylsulfonyl)-[3,3′-bipyridine]-1,1′-diium bis(tetrafluoroborate) (CAP6)

A portion of 6-chloro-6′-(methylsulfonyl)-3,3′-bipyridine (20 mg, 0.074 mmol) and trimethyloxonium tetrafluoroborate (46.1 mg, 0.312 mmol) were mixed with anhydrous CH₃NO₂ (200 μL) in a vial in the glovebox. The reaction was stirred at room temperature for 36 hours. The reaction mixture was diluted with anhydrous CH₃CN (3 mL) and filtered through a cotton plug. The filtrate was diffused with anhydrous CH₂Cl₂. After six days, the off-white crystals were collected, washed with anhydrous Et₂O, and dried under vacuum (26 mg, 74%).

¹H NMR (400 MHz, CD₃CN) δ 9.31 (d, J=2.0 Hz, 1H), 9.23 (d, J=2.2 Hz, 1H), 9.06 (dd, J=8.4, 2.0 Hz, 1H), 8.86 (d, J=8.4 Hz, 1H), 8.82 (dd, J=8.7, 2.3 Hz, 1H), 8.37 (d, J=8.7 Hz, 1H), 4.74 (s, 3H), 4.43 (s, 3H), 3.55 (s, 3H). ¹³C{¹H} NMR (101 MHz, CD₃CN) δ 151.93, 150.88, 150.29, 148.05, 147.33, 146.65, 137.59, 132.01, 131.84, 131.61, 49.47, 49.29, 44.46. ESI-HRMS(+) m/z calculated for C₁₃H₁₅ClN₂O₂S²⁺[M]²⁺149.0266, found 149.0236.

### Example 7: 2-Chloro-1,1′-dimethyl-2′-(methylsulfonyl)-[4,4′-bipyridine]-1,1′-diium bis(tetrafluoroborate) (CAP7)

A portion of 2-chloro-2′-(methylsulfonyl)-4,4′-bipyridine (26.9 mg, 0.1 mmol) and trimethyloxonium tetrafluoroborate (35.9 mg, 0.243 mmol) were mixed with anhydrous CH₃NO₂ (100 μL) in a vial in the glovebox. The reaction was stirred at room temperature for 24 hours. The reaction mixture was diluted with anhydrous CH₃CN (2 mL) and filtered through a cotton plug. The filtrate was diffused with anhydrous Et₂O. After ten days, an off-white crystalline powder was collected, washed with anhydrous Et₂O, and dried under vacuum (20 mg, 42%).

¹H NMR (400 MHz, CD₃CN) δ 9.13 (d, J=6.3 Hz, 1H), 9.02-8.94 (m, 2H), 8.67 (q, J=3.0 Hz, 2H), 8.41 (dd, J=6.6, 2.2 Hz, 1H), 4.71 (s, 3H), 4.40 (s, 3H), 3.57 (s, 3H). ¹³C{¹H} NMR (101 MHz, CD₃CN) δ 152.58, 152.49, 152.14, 150.86, 150.06, 149.78, 131.50, 130.33, 130.01, 126.05, 49.25, 48.92, 44.35. ESI-HRMS(+) m/z calculated for C₁₃H₁₅ClN₂O₂S²⁺[M]²⁺149.0266, found 149.0223.

### Example 8: 2-Chloro-1,1′-dimethyl-2′-(methylsulfonyl)-[3,3′-bipyridine]-1,1′-diium bis(tetrafluoroborate) (CAP8)

A portion of 2-chloro-2′-(methylsulfonyl)-3,3′-bipyridine (134 mg, 0.5 mmol) and trimethyloxonium tetrafluoroborate (178 mg, 1.2 mmol) were mixed with anhydrous CH₃NO₂ (330 μL) in a vial in the glovebox. The reaction was stirred at room temperature for 24 hours. The reaction mixture was diluted with anhydrous CH₃CN (3 mL) and filtered through a cotton plug. The filtrate was diffused with anhydrous CH₂Cl₂. After seven days, an oil residue formed. This oil residue was separated from the solvents and dissolved in anhydrous CH₃CN (5 mL) and diffused with CH₂Cl₂. After ten days, a white crystalline powder was collected, washed with anhydrous Et₂O, and dried under vacuum (183 mg, 77%).

¹H NMR (400 MHz, CD₃CN) δ 9.14 (d, J=6.0 Hz, 1H), 8.90 (d, J=6.3 Hz, 1H), 8.58 (d, J=8.3,Hz, 1H), 8.53-8.39 (m, 2H), 8.10 (dd, J=8.1, 6.2 Hz, 1H), 4.77 (s, 3H), 4.41 (s, 3H), 3.51 (s, 3H). ¹³C{¹H} NMR (101 MHz, CD₃CN) δ 154.00, 151.84, 149.99, 149.57, 147.59, 147.24, 137.67, 137.40, 133.31, 127.02, 51.30, 49.80, 45.93. ESI-HRMS(+) m/z calculated for C₁₃H₁₅ClN₂O₂S²⁺[M]²⁺149.0266, found 149.0234.

### Example 9: N-Methyl-2-fluoro-6-(methylsulfonyl)pyridinium tetrafluoroborate (CAP9)

A portion of 2-fluoro-6-(methylsulfonyl)pyridine (175 mg, 1 mmol) and trimethyloxonium tetrafluoroborate (222 mg, 1.5 mmol) were mixed in anhydrous CH₃NO₂ (100 μL) in a glovebox. The reaction was stirred at room temperature for 36 h. The reaction mixture was diluted with anhydrous MeCN (4 mL) and filtered through a 0.2 μm PTFE syringe filter. The filtrate was layered with Et₂O (25 mL) at −35° C. After seven days, the white crystals were collected and washed with Et₂O (232 mg, 65% yield).

¹H NMR (400 MHz, CD₃CN) δ 8.87 (td, J=8.3, 5.8 Hz, 1H), 8.57 (d, J=7.9 Hz, 1H), 8.14 (dd, J=8.7, 4.4 Hz, 1H), 4.45 (d, J=4.2 Hz, 3H), 3.51 (s, 3H). ¹³C{¹H} NMR (101 MHz, CD₃CN) δ 161.54 (d, J=286.6 Hz), 153.44 (d, J=13.6 Hz), 149.86 (d, J=7.9 Hz), 128.06 (d, J=2.0 Hz), 120.80 (d, J=22.7 Hz), 44.52 (s), 39.75 (d, J=8.7 Hz). ¹⁹F NMR (377 MHz, CD₃CN) δ −68.44−68.55 (m, 1F), −151.89 (s, ¹⁰BF₄⁻), −151.94 (s, ¹¹BF₄⁻). ESI-HRMS(+) m/z calculated for C₇H₉FNO₂S⁺[M]⁺190.0333, found 190.0330.

### Example 10: 6,6′-Difluoro-1,1′-dimethyl-[3,3′-bipyridine]-1,1′-diium bis(tetrafluoroborate) (CAP10)

In a glovebox, 6,6′-difluoro-3,3′-bipyridine (192 mg, 1 mmol) and trimethyloxonium tetrafluoroborate (325 mg, 2.2 mmol) were mixed in anhydrous MeCN (6 mL). The reaction was stirred at rt for 24 h, which led to the formation of a large amount white solid. The reaction mixture was dissolved in MeCN (8 mL) and filtered through a cotton plug. The filtrate was layered with Et₂O (25 mL). After a week, colorless crystals formed. After seven days, the crystals were collected and washed with anhydrous Et₂O (281 mg, 71% yield).

¹H NMR (400 MHz, CD₃CN) δ 8.95-8.87 (m, 4H), 8.02 (dd, J=8.7, 3.8 Hz, 2H), 4.29 (d, J=3.8 Hz, 6H). ¹³C¹{H} NMR (101 MHz, CD₃CN) δ 160.61 (d, J=282.5 Hz), 150.88 (d, J=12.2 Hz), 144.58 (d, J=6.7 Hz), 130.62 (d, J=3.8 Hz), 116.28 (d, J=21.2 Hz), 43.43 (d, J=5.2 Hz). ¹⁹F NMR (377 MHz, CD₃CN) δ −77.00 (dt, J=7.7, 3.9 Hz, 2F),−151.45 (s, ¹⁰BF₄⁻), −151.50 (s, ¹¹BF₄⁻).

### Example 11: 6,6′-Difluoro-1,1′-dimethyl-[2,2′-bipyridine]-1,1′-diium bis(tetrafluoroborate) (CAP11)

In a glovebox, 6,6′-difluoro-2,2′-bipyridine (67 mg, 0.35 mmol) and trimethyloxonium tetrafluoroborate (123 mg, 2.4 mmol) were mixed in anhydrous MeNO₂ (150 μL). The reaction was stirred at rt for 36 h. The reaction mixture was dissolved in MeCN (10 mL) and filtered through a cotton plug. Anhydrous Et₂O was diffused into the filtrate. After a week, colorless crystals formed. After seven days, the crystals were collected and washed with anhydrous Et₂O (61.9 mg, 45% yield).

¹H NMR (400 MHz, CD₃CN) δ 8.89 (td, J=8.2, 6.0 Hz, 2H), 8.18 (ddd, J=8.8, 4.3, 0.8 Hz, 2H), 8.11 (d, J=7.7 Hz, 2H), 3.97 (d, J=4.1 Hz, 6H). ¹³C NMR (101 MHz, CD₃CN) δ 161.77 (d, J=284.8 Hz), 153.53 (d, J=13.6 Hz), 141.22 (d, J=7.4 Hz), 129.29 (d, J=2.7 Hz), 119.43 (d, J=21.6 Hz), 40.85 (d, J=7.1 Hz).

### Example 12: 6,6′-Dimethylsulfonyl-1,1′-dimethyl-[3,3′-bipyridine]-1,1′-diium bis(tetrafluoroborate) (CAP12)

In a glovebox, 6,6′-dimethylsulfonyl-3,3′-bipyridine (250 mg, 0.8 mmol) and trimethyloxonium tetrafluoroborate (355 mg, 2.4 mmol) were mixed in anhydrous CH₃NO₂ (380 μL). The reaction was stirred at rt for 72 h to form a white slurry. The slurry was washed with anhydrous MeCN (1 mL×5) and anhydrous Et₂O (1 mL). The solid was dried under vacuum to give a white powder (117 mg, 28% yield).

¹H NMR (400 MHz, CD₃CN) δ 9.35 (d, J=1.4 Hz, 2H), 9.12 (dd, J=8.4, 1.8 Hz, 2H), 8.91 (d, J=8.4 Hz, 2H), 4.76 (s, 6H), 3.57 (s, 6H). ¹³C NMR (101 MHz, CD₃CN) δ 152.57, 150.66, 148.00, 137.07, 132.23, 49.66, 44.55.

### Example 13: 1-Methyl-2-(((trifluoromethyl)sulfonyl)oxy)pyridin-1-ium tetrafluoroborate (CAP13)

In a glovebox, 2-pyridyl trifluoromethanesulfonate (909 mg, 615 μL, 4 mmol) and trimethyloxonium tetrafluoroborate (592 mg, 4 mmol) were added to a vial. The reaction was stirred at rt for 48 h. The formed slurry was dissolved in MeCN (6 mL) and filtered through a cotton plug. The filtrate was layered with Et₂O (25 mL). After ten days, colorless needles formed along with fine amorphous opaque material. The crystals were washed with Et₂O. The amorphous material was removed by pipetting. The crystals were collected and dried under vacuum (894 mg, 68% yield).

¹H NMR (400 MHz, CD₃CN) δ 8.80-8.66 (m, 2H), 8.14-7.98 (m, 2H), 4.25 (s, 3H). ¹³C¹{H} NMR (101 MHz, CD₃CN) δ 151.81 (s), 150.85 (s), 148.51 (s), 127.92 (s), 120.43 (s), 119.36 (q, J=320.6 Hz), 45.32 (s). ¹⁹F NMR (376 MHz, CD₃CN) δ −72.91 (s, 3F), −151.89 (s, ¹⁰BF₄⁻), −151.94 (s, ¹¹BF₄⁻).

### Example 14: N-Methyl-2-fluoro-5-nitropyridinium tetrafluoroborate (CAP14)

In a glovebox, 2-fluoro-5-nitropyridine (284 mg, 2 mmol) and Me₃OBF₄ (311 mg, 2.1 mmol) were mixed in anhydrous MeCN (4 mL). The reaction was stirred at room temperature for 48 h. The reaction mixture was filtered through a cotton plug and diluted with MeCN (6 mL). The filtrate was layered with Et₂O (about 25 mL). After seven days, colorless needles were collected and washed with Et₂O (145 mg, 30% yield).

¹H NMR (400 MHz, CD₃CN) δ 9.52 (pseudo t, J=3.2 Hz, 1H), 9.29 (ddd, J=9.3, 4.5, 2.8 Hz, 1H), 8.05 (dd, J=9.4, 3.5 Hz, 1H), 4.29 (d, J=3.9 Hz, 1H). ¹³C¹{H}NMR (101 MHz, CD₃CN) δ 162.72 (d, J=289.9 Hz), 146.68 (d, J=13.7 Hz), 144.16 (d, J=3.6 Hz), 143.52 (d, J=5.2 Hz), 116.75 (d, J=22.6 Hz), 44.24 (d, J=5.1 Hz). ¹⁹F NMR (377 MHz, CD₃CN) δ-68.65 (ddq, J=7.6, 3.8, 3.8 Hz, 1F), −151.91 (s, ¹⁰BF₄⁻), −151.96 (s, ¹¹BF₄⁻).

### Example 15: N-Methyl-2-chloro-5-nitropyridinium tetrafluoroborate (CAP15)

In a glovebox, 2-chloro-5-nitropyridine (634 mg, 4 mmol) and Me₃OBF₄ (651 mg, 4.4 mmol) were mixed in anhydrous MeCN (4 mL). The reaction was stirred at room temperature for 24 h. The reaction mixture was filtered through a cotton plug and diluted with MeCN (4 mL). The filtrate was layered with Et₂O (about 30 mL). After seven days, slightly brown needles were collected and washed with Et₂O (724 mg, 70% yield).

¹H NMR (400 MHz, CD₃CN) δ 9.70 (d, J=2.5 Hz, 1H), 9.12 (dd, J=9.0, 2.5 Hz, 1H), 8.37 (d, J=9.1 Hz, 1H), 4.43 (s, 3H). ¹³C¹{H} NMR (101 MHz, CD3CN) δ 154.83 (s), 146.03 (s), 145.86 (s), 142.09 (s), 131.99 (s), 49.92 (s).

### Example 16: 3,6-Dichloro-1-methylpyridazin-1-ium tetrafluoroborate (CAP16)

In a glovebox, 3,6-dichloropyridazine (447 mg, 3 mmol) and Me₃OBF₄ (887 mg, 6 mmol) were mixed in anhydrous MeCN (2 mL). The reaction was stirred at room temperature for 24 h. The reaction mixture was filtered through a cotton plug and diluted with MeCN (4 mL). The filtrate was layered with Et₂O (about 25 mL). After seven days, a mixture of brown oil and colorless crystals formed. The oil was carefully removed using a pipette. The remaining oil on the crystals were quickly washed away using a small amount of anhydrous acetone (2 mL×2). The crystals were then washed with anhydrous Et₂O (3 mL×4) and dried under vacuum. Colorless blocks were obtained (294 mg, 39% yield).

¹H NMR (400 MHz, CD₃CN) δ 8.53 (d, J=9.1 Hz, 1H), 8.46 (dq, J=9.1, 0.5 Hz, 1H), 4.56 (s, 3H). ¹³C¹{H} NMR (101 MHz, CD₃CN) δ 157.42 (s), 156.05 (s), 140.90 (s), 140.42 (s), 52.85 (s). ¹⁹F NMR (376 MHz, CD₃CN) δ −152.05 (s, ¹⁰BF₄⁻), −152.10 (s, ¹¹BF₄).

### Example 17: 2-Chloro-1,3-dimethylpyrimidine-1,3-diium bis(tetrafluoroborate) (CAP17)

In a glovebox, 2-chloropyrimidine (115 mg, 1 mmol) and Me₃OBF₄ (325 mg, 2.2 mmol) were mixed in anhydrous MeNO₂ (100 μL). The reaction was stirred at room temperature for 72 h. The reaction mixture was diluted with anhydrous MeCN (8 mL) and filtered through a cotton plug. The filtrate was layered with Et₂O (about 25 mL). After seven days, a mixture of orange oil and colorless crystals formed. The oil was separated from the crystals using a pipette. The crystals, contaminated with a small amount of the orange oil, were dissolved in anhydrous MeCN and crystalized by diffusing Et₂O into this solution. After seven days, colorless crystals formed along with a small amount of oil on the surface. The crystals were collected and dissolved in anhydrous MeCN. Et₂O was diffused into this solution. After seven days, formed slightly yellow crystals were collected and washed with anhydrous Et₂O (41 mg, blocks, 13% yield).

¹H NMR (400 MHz, CD₃CN) δ 8.63 (d, J=6.3 Hz, 1H), 6.84 (t, J=6.3 Hz, 1H), 3.72 (s, 3H). ¹³C¹{H} NMR (101 MHz, CD₃CN) δ 161.47 (s), 148.99 (s), 104.46 (s), 41.56 (s). ¹⁹F NMR (376 MHz, CD₃CN) δ −151.80 (s, ¹⁰BF₄), −151.85 (s, ¹¹BF₄⁻).

### Example 18: N-Methyl-2,4-difluoropyridinium tetrafluoroborate (CAP18)

In a glovebox, 2,4-difluoropyridine (230 mg, 182 μL, 2 mmol) and Me₃OBF₄ (443.7 mg, 3 mmol) were mixed in anhydrous MeCN (500 μL). The reaction was stirred at room temperature for 24 h. The reaction mixture was diluted with anhydrous MeCN (4 mL) and filtered through a cotton plug. The filtrate was layered with Et₂O (25 mL) and stored at −40° C. After seven days, colorless blocks formed and were collected. The crystals were washed with Et₂O (4 mL×3) and dried under vacuum (420 mg, 91% yield).

¹H NMR (400 MHz, CD₃CN) δ 8.61 (dd, J=12.9, 5.7 Hz, 1H), 7.75-7.63 (m, 2H), 4.13 (d, J=3.8 Hz, 3H). ¹³C NMR (101 MHz, CD₃CN) δ 176.15 (dd, J=280.5, 14.2 Hz), 162.22 (dd, J=277.6, 18.8 Hz), 148.51 (br dd, J=13.5, 5.6 Hz), 114.58 (dd, J=22.8, 3.0 Hz), 104.45 (dd, J=26.7, 24.5 Hz), 42.54 (d, J=5.1 Hz). ¹⁹F NMR (377 MHz, CD₃CN) δ −72.81 (dtq, J=30.0, 8.0, 3.9 Hz), −75.86 (dq, J=29.6, 6.3 Hz), −151.78 (s, ¹⁰BF₄⁻), −151.83 (s, ¹¹BF₄⁻).

### Example 19: N-Ethyl-2,4-difluoropyridinium tetrafluoroborate (CAP19)

In a glovebox, 2,4-difluoropyridine (230 mg, 182 μL, 2 mmol) and Et₃OBF₄ (399 mg, 2.1 mmol) were mixed in anhydrous CH₂Cl₂ (1 mL). The reaction was stirred at room temperature for 24 h. A large amount of white solid formed. The solvent was removed by pipette. The solid was dissolved in anhydrous MeCN (4 mL) and filtered through a cotton plug. The filtrate was layered with Et₂O (25 mL). After three days, a small amount of crystals formed along with a large amount of colorless oil. The mixture was stored at −40° C. for four hours. The oil solidified. The solvents were quickly removed by pipette. The white crystalline solid was washed with anhydrous Et₂O and dried under vacuum (356 mg, 77% yield).

¹H NMR (400 MHz, CD₃CN) δ 8.65 (dt, J=6.9, 5.6 Hz, 1H), 7.76-7.65 (m, 2H), 4.55 (qd, J=7.3, 2.5 Hz, 2H), 1.55 (t, J=7.3 Hz, 3H). ¹³C NMR (101 MHz, CD₃CN) δ 176.13 (dd, J=279.5, 13.3 Hz), 161.98 (br d, J=277.2 Hz), 147.32 (dd, J=13.1, 5.1 Hz), 115.09 (dd, J=22.8, 3.0 Hz), 104.90 (dd, J=26.8, 24.9 Hz), 52.52 (d, J=4.5 Hz), 14.81 (s). ¹⁹F NMR (377 MHz, CD₃CN) δ −75.31 (dtt, J=29.6, 4.8, 2.2 Hz, 1F),−75.87 (pseudo dq, J=29.6, 6.2 Hz, 1F),−151.79 (s, ¹⁰BF₄⁻),−151.84 (s, ¹¹BF₄⁻).

### Example 20: 2-Chloro-3-methylbenzothiazol-3-ium tetrafluoroborate (CAP20)

In a glovebox, 2-chlorobenzothiazole (678 mg, 4 mmol) and Me₃OBF₄ (651 mg, 4.4 mmol) were mixed in anhydrous MeCN (4 mL). The reaction was stirred at room temperature for 24 h. The reaction mixture was diluted with anhydrous MeCN (4 mL) and filtered through a cotton plug. The filtrate was layered with Et₂O (30 mL). After seven days, the white crystals were collected and washed with Et₂O (815 mg, 75% yield).

¹H NMR (400 MHz, CD₃CN) δ 8.26 (ddd, J=8.2, 1.2, 0.6 Hz, 1H), 8.11 (d, J=8.4 Hz, 1H), 7.95 (ddd, J=8.6, 7.4, 1.2 Hz, 1H), 7.87 (ddd, J=8.4, 7.4, 1.1 Hz, 1H), 4.24 (s, 3H). ¹³C¹{H} NMR (101 MHz, CD₃CN) δ 165.40 (s), 141.44 (s), 131.53 (s), 130.66 (s), 130.00 (s), 125.15 (s), 118.14 (s), 38.47 (s). ¹⁹F NMR (377 MHz, CD₃CN) δ −151.70 (s, ¹⁰BF₄⁻), −151.75 (s, ¹¹BF₄⁻).

### Example 21:1-Ethyl-2-(9-azido-1,4,7-trioxanonanyl)-4-methylsulfonyl pyridinium tetrafluoroborate (CAP21)

A portion of triethyloxonium tetrafluoroborate (16 mg, 0.084 mmol) were added to a solution of 1-azido-9-(4-methylsulfonyl-2-pyridyl)-3,6,9-trioxanonane (26.4 mg, 0.080 mmol) in CH₂Cl₂ (320 μL). The reaction was stirred at r.t. for 24 h. The solvent was removed under vacuum. A sticky liquid was obtained (35.3 mg, 99%).

¹H NMR (400 MHz, CD₂Cl₂) δ 8.58 (d, J=6.6 Hz, 1H), 7.99 (d, J=5.3 Hz, 1H), 7.84 (dd, J=6.6, 1.4 Hz, 1H), 4.87-4.71 (m, 2H), 4.54 (q, J=7.2 Hz, 2H), 4.05-3.94 (m, 2H), 3.77-3.54 (m, 6H), 3.37-3.30 (m, 2H), 3.25 (s, 3H), 1.55 (t, J=7.3 Hz, 3H). ¹³C NMR (101 MHz, CD₂Cl₂) δ 161.44, 157.43, 144.85, 116.65, 111.98, 73.99, 71.06, 70.73, 70.18, 68.91, 52.47, 51.04, 43.22, 14.29. ESI-MS(+) m/z calculated for [M+H]⁺ 359.1, found 359.2. ESI-HRMS(+) m/z calculated for C₁₄H₂₃N₄O₅S⁺[M]⁺359.1384, found 359.1378.

### Example 22:1-Ethyl-4-(9-azido-1,4,7-trioxanonanyl)-2-methylsulfonyl pyridinium tetrafluoroborate (CAP22)

A portion of triethyloxonium tetrafluoroborate (20 mg, 0.105 mmol) were added to a solution of 1-azido-9-(2-methylsulfonyl-4-pyridyl)-3,6,9-trioxanonane (33 mg, 0.1 mmol) in CH₂Cl₂ (400 μL). The reaction was stirred at r.t. for 24 h. The solvent was removed under vacuum. A sticky liquid was obtained (44.1 mg, 99%).

¹H NMR (400 MHz, CD₂Cl₂) δ 8.71 (d, J=7.1 Hz, 1H), 8.07 (d, J=2.8 Hz, 1H), 7.66 (dd, J=7.1, 2.8 Hz, 1H), 4.83 (q, J=7.1 Hz, 2H), 4.60-4.56 (m, 2H), 3.96-3.92 (m, 2H), 3.70-3.60 (m, 6H), 3.46 (s, 3H), 3.35 (t, J=4.8 Hz, 2H), 1.64 (t, J=7.1 Hz, 3H). ¹³C NMR (101 MHz, CD₂Cl₂) δ 172.66, 152.15, 150.42, 118.68, 116.47, 72.10, 71.15, 70.76, 70.21, 69.09, 55.04, 51.07, 44.68, 17.26. ESI-MS(+) m/z calculated for [M+H]⁺ 359.1, found 359.3. ESI-HRMS(+) m/z calculated for C₁₄H₂₃N₄O₅S⁺[M]⁺359.1384, found 359.1393.

### Example 23: 1,3-dimethyl-2-(methylsulfonyl)-1H-benzo[d]imidazol-3-ium tetrafluoroborate (CAP23)

A portion of N-methyl-2-methylsulfonebenzoimidazole (52.6 mg, 0.25 mmol) was mixed with trimethyloxonium tetrafluoroborate (44.3, 0.3 mmol) in anhydrous nitromethane (25 μL) in a glovebox. The mixture was stirred at r.t. for 24 h, diluted with anhydrous acetonitrile (3 mL), and filtered through a cotton plug. The desired product was crystallized by diffusing anhydrous Et₂O into the above filtrate. After seven days, clear crystals were obtained (47.6 mg, 61%).

1H NMR (400 MHz, D2O) δ 8.08−7.98 (m, 2H), δ 7.92−7.85 (m, 2H), 4.41 (s, 6H), 3.75 (s, 3H). The compound exhibits moderate stability in D₂O and completely hydrolyzes at r.t. after 12 h.]

### Example 24: 1,1′-(1,4-phenylenebis(methylene))bis(3-fluoropyridin-1-ium) diiodide (CAP24)

A mixture of 3-fluoropyridine (258 μL, 3 mmol) and 1,4-bis(bromomethyl)-benzene (264 mg, 1 mmol) in anhydrous acetonitrile (1 mL) was heated at 80° C. for 72 h. The formed solid was suspended in MeCN (2 mL) and stirred for 30 min. The solid was isolated by suction filtration and washed with MeCN. The product was dried under vacuum to give a white solid (450 mg, 98% yield).

¹H NMR (400 MHz, D₂O) δ 9.11-8.99 (m, 2H), 8.87 (d, J=6.0 Hz, 2H), 8.51-8.36 (m, 2H), 8.20-8.07 (m, 2H), 7.57 (d, J=0.4 Hz, 4H), 5.90 (s, 4H). ¹⁹F NMR (376 MHz, D₂O) δ −114.7 (ddd, J=7.1, 5.4, 3.4 Hz).

### Example 25: N-Methyl-3-(methylsulfonyl)pyridinium tetrafluoroborate (CAP25)

3-(Methylsulfonyl)pyridinium tetrafluoroborate is synthesized by methylating known compound 3-(methylsulfonyl)pyridine with trimethyloxonium tetrafluoroborate in anhydrous nitromethane. The reaction is carried out under conditions similar to that for preparing pyridinium salts described above.

### Example 26: N-Methyl-3-(N′,N′-dimethylphenylsulfoximinium) pyridinium bis(tetrafluoroborate) (CAP26)

CAP26 is synthesized via two steps starting with a known compound, 3-(phenylsulfoximine) pyridine (see Org. Lett. 2020, 22, 2776). The imine nitrogen of 3-(phenylsulfoximine) pyridine is methylated with trimethyloxonium tetrafluoroborate to give 3-(N′-methylphenylsulfoximine)pyridine (see J. Fluorine Chem. 2011, 132, 792). This synthetic intermediate, 3-(N′-methylphenylsulfoximine)pyridine, is further methylated with excess trimethyloxonium tetrafluoroborate in anhydrous nitromethane under conditions similar to those for preparing pyridinium salts described above, generating the desired dicationic salt.

Following a similar procedure, CAP27 can also be prepared using commercially available reagents.

### Example 27: Labeling Glutathione (GSH) Using CAP1-a

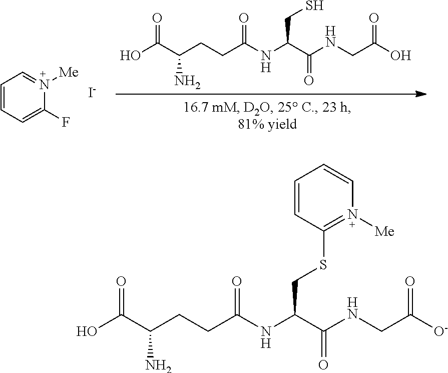

A stock solution of CAP1-a (2.39 mg, 0.01 mmol) in D₂O (300 μL, 33.3 mM) and GSH (3.09 mg, 0.01 mM) in D₂O (300 μL, 33.3 mM) were mixed and kept at 25° C. The progress of the reaction was monitored by ¹H NMR spectroscopy at 25° C. According to the ¹H NMR integration, the GSH-CAP1-a was obtained in 81% yield after 23 h.

¹H NMR (400 MHz, D₂O) δ 8.68 (dd, J=6.7, 5.8 Hz, 1H), 8.32 (dt, J=7.4, 1.3 Hz, 1H), 8.04 (d, J=8.2 Hz, 1H), 7.69 (ddd, J=7.6, 6.4, 1.2 Hz, 1H), 4.85 (dd, J=7.9, 5.6 Hz, 1H), 4.19 (s, 3H), 3.98 (s, 1H), 3.97 (s, 1H), 3.91 (dd, J=14.2, 5.6 Hz, 1H), 3.86 (t, J=6.5 Hz, 1H), 3.70 (dd, J=14.2, 8.0 Hz, 1H), 2.51 (dd, J=8.9, 6.3 Hz, 2H), 2.23-2.07 (m, 2H). ¹³C¹{H} NMR (101 MHz, D₂O) δ 174.44, 172.87, 171.94, 171.06, 158.43, 146.85, 143.72, 125.50, 123.02, 52.56, 51.62, 46.21, 41.23, 33.62, 30.96, 25.47. MS (ESI+): m/z calculated for M⁺ 399.1, found 399.1.

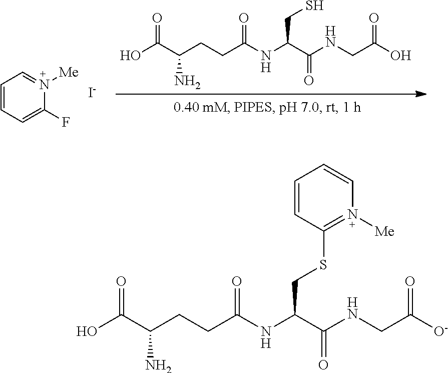

The reaction was performed again under the procedure as follows. To a mixture of 50 mM PIPES buffer (40 μL, pH 7) and water (355 μL), an aliquot of GSH (1.6 μL, 100 mM in H₂O) was added. To this mixture, CAP1-a (3.2 μL, 50 mM in H₂O) was added. The mixture was kept at room temperature for 1 h and then purified by HPLC using solvent gradient 2 (Table 3, semi prep HPLC T_{R}=12.31 min) to obtain a white solid as a trifluoroacetic acid salt. Analytical HPLC using solvent gradient 11, T_{R}=3.37 min.

MS (ESI+): m/z calculated for M+399.1, found 399.1. ESI-HRMS(+) m/z calculated for C₁₆H₂₃N₄O₆S⁺ M⁺ 399.1333, found 399.1320. The structure of the product was also confirmed by tandem MS study.

### Example 28: Labeling GSH Using CAP1-N₃

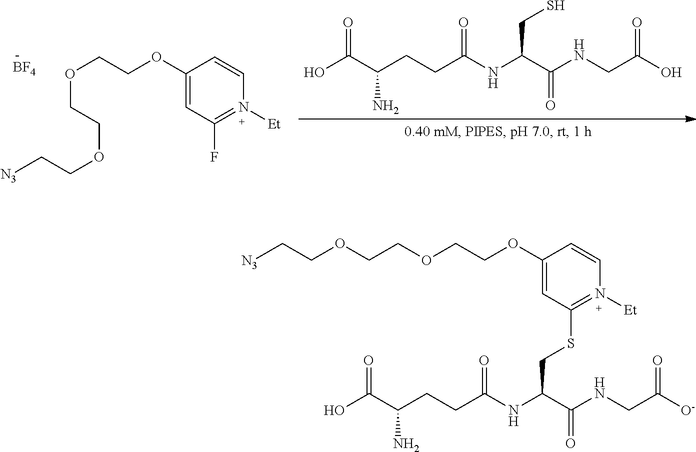

To a mixture of 50 mM PIPES buffer (40 μL, pH 7) and water (355 μL), an aliquot of GSH (1.6 μL, 100 mM in H₂O) was added. To this mixture, CAP1-N₃ (3.2 μL, 50 mM in H₂O) was added. The mixture was kept at room temperature for 1 h. The reaction mixture was purified by HPLC using solvent gradient 3 (Table 3, semi prep HPLC T_{R}=8.48 min) to obtain a white solid as a trifluoroacetic acid salt. Analytical HPLC using solvent gradient 11, T_{R}=11.97 min.

MS (ESI+): m/z calculated for M⁺586.2, found 586.4. ESI-HRMS(+) m/z calculated for C₂₃H₃₆N₇O₉S⁺ M⁺586.2290, found 586.2276. The structure of the product was also confirmed by tandem MS study.

### Example 29: Labeling GSH Using CAP1-CCH

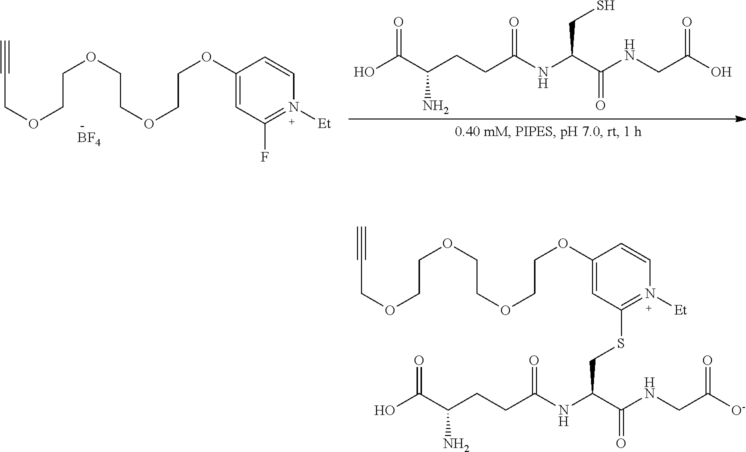

To a mixture of 50 mM PIPES buffer (40 μL, pH 7) and water (355 μL), an aliquot of GSH (1.6 μL, 100 mM in H₂O) was added. To this mixture, CAP1-CCH (3.2 μL, 50 mM in H₂O) was added. The mixture was kept at room temperature for 1 h. The reaction mixture was purified by HPLC using solvent gradient 3 (Table 3, semi prep HPLC T_{R}=8.50 min) to obtain a white solid as a trifluoroacetic acid salt. Analytical HPLC using solvent gradient 11, T_{R}=11.99 min.

MS (ESI+): m/z calculated for M⁺599.2, found 599.4. ESI-HRMS(+) m/z calculated for C₂₆H₃₉N₄O₁₀S⁺ M⁺599.2382, found 599.2372. The structure of the product was also confirmed by tandem MS study.

### Example 30: One-Pot Two-Step Labeling of GSH Using CAP1-F and Phenol in PIPES Buffer

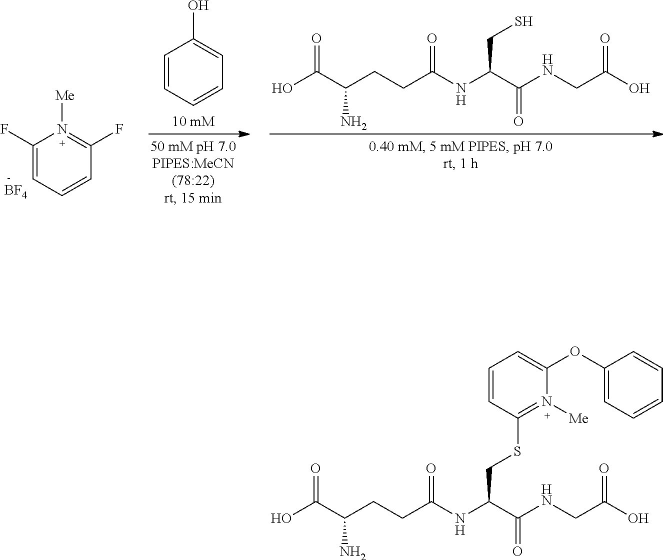

To a mixture of PIPES buffer (31.6 μL, pH 7) and PhOH (4 μL, 100 mM in MeCN), an aliquot of CAP1-F (4.4 μL, 100 mM in MeCN) was added. This mixture was kept at room temperature for 15 min. An aliquot of this reaction mixture (16 μL) was added to a mixture of GSH (1.6 μL, 100 mM in H₂O), 50 mM PIPES buffer (40 μL, pH 7), and water (342 μL). The mixture was kept at room temperature for 1 h. The reaction mixture was purified by HPLC using solvent gradient 4 (Table 3, semi prep HPLC T_{R}=8.67 min) to obtain a white solid as a trifluoroacetic acid salt. Analytical HPLC using solvent gradient 11, T_{R}=11.94 min.

MS (ESI+): m/z calculated for M⁺ 491.2, found 491.2. ESI-HRMS(+) m/z calculated for C₂₂H₂₇N₄O₇S⁺ M⁺491.1595, found 491.1606. The structure of the product was also confirmed by tandem MS study.

### Example 31: One-Pot Two-Step Labeling of GSH Using CAP1-F and Phenol with DIPEA in MeCN and then in PIPES Buffer

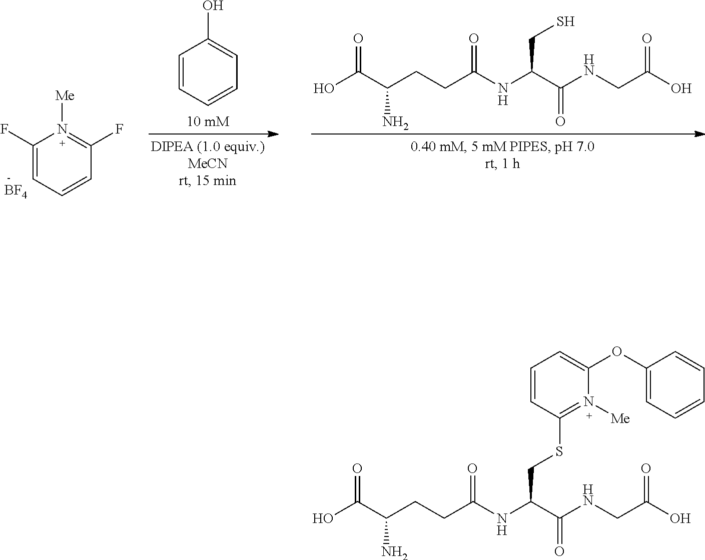

To a mixture of DIPEA (50 μL, 100 mM in MeCN) and PhOH (50 μL, 100 mM in MeCN) in MeCN (350 μL), an aliquot of CAP1-F (50 μL, 100 mM in MeCN) was added. This mixture was kept at room temperature for 20 min. An aliquot of this reaction mixture (16 μL) was added to 50 mM PIPES buffer (40 μL). The mixture was diluted with water (342 μL). An aliquot of GSH (1.6 μL, 100 mM in H₂O) was then added to this mixture. The mixture was kept at room temperature for 1 h. The reaction mixture was purified by HPLC using solvent gradient 4 (Table 3, semi prep HPLC T_{R}=8.67 min) to obtain a white solid as a trifluoroacetic acid salt.

MS (ESI+): m/z calculated for M⁺ 491.2, found 491.2.

### Example 32: One-Pot Two-Step Labeling of GSH Using CAP1-F and Phenol with DIPEA in MeCN and DMF and then in PIPES Buffer

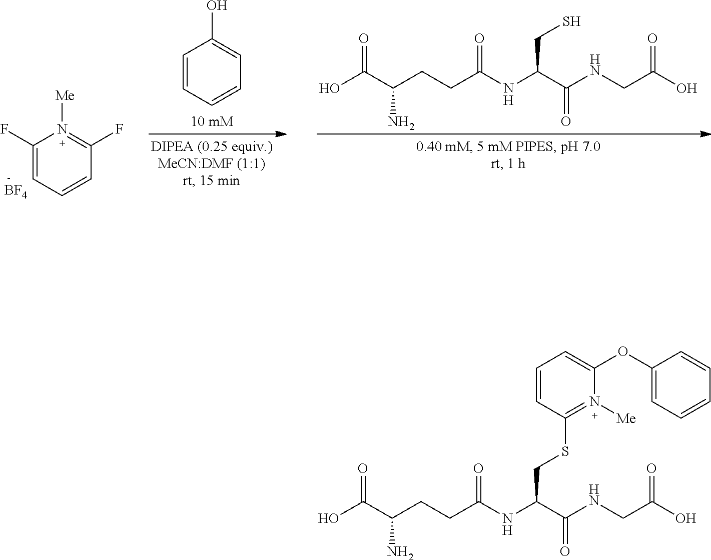

To a mixture of DIPEA (12.5 μL, 100 mM in MeCN) and PhOH (50 μL, 100 mM in MeCN) in MeCN (137.5 μL) and DMF (250 μL), an aliquot of CAP1-F (50 μL, 100 mM in MeCN) was added. This mixture was kept at room temperature for 20 min. An aliquot of this reaction mixture (16 μL) was added to 50 mM PIPES buffer (40 μL). The mixture was diluted with water (342 μL). An aliquot of GSH (1.6 μL, 100 mM in H₂O) was then added to this mixture. The mixture was kept at room temperature for 1 h. The reaction mixture was purified by HPLC using solvent gradient 4 (Table 3, semi prep HPLC T_{R}=8.67 min) to obtain a white solid as a trifluoroacetic acid salt. MS (ESI+): m/z calculated for M⁺ 491.2, found 491.2.

### Example 33: One-Pot Two-Step Labeling of GSH Using CAP1-F and Tetramethylrhodamine Phenol Derivative in PIPES Buffer

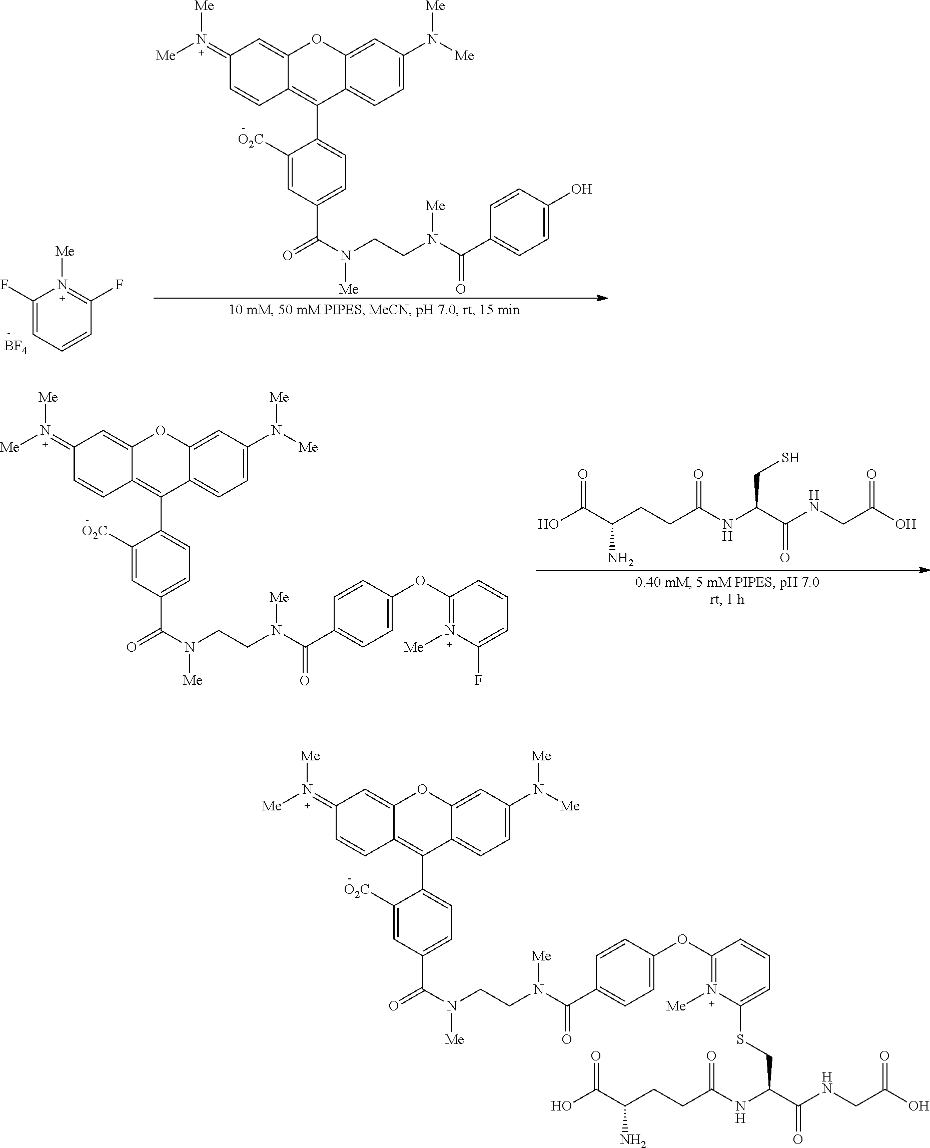

To a mixture of PIPES buffer (24.2 μL, pH 7) and tetramethylrhodamine phenol derivative (11.4 μL, 35 mM in MeCN), an aliquot of CAP1-F (4.4 μL, 100 mM in MeCN) was added. This mixture was kept at room temperature for 15 min. An aliquot of this reaction mixture (16 μL) was added to a mixture of GSH (1.6 μL, 100 mM in H₂O), 50 mM PIPES buffer (40 μL, pH 7), and water (342 μL). The reaction mixture was purified by HPLC using solvent gradient 5 (Table 3, semi prep HPLC T_{R}=12.13 min) to obtain a purple solid as a trifluoroacetic acid salt. Analytical HPLC using solvent gradient 11, T_{R}=18.08 min.

MS (ESI+): m/z calculated for [M+H]²⁺509.2, found 509.6. ESI-HRMS(+) m/z calculated for C₅₂H₅₈N₈O₁₂S²⁺ M⁺509.1942, found 509.1931. The structure of the product was also confirmed by tandem MS study.

### Example 34: One-Pot Two-Step Labeling of GSH Using CAP1-F and Biotin Phenol Derivative in PIPES Buffer

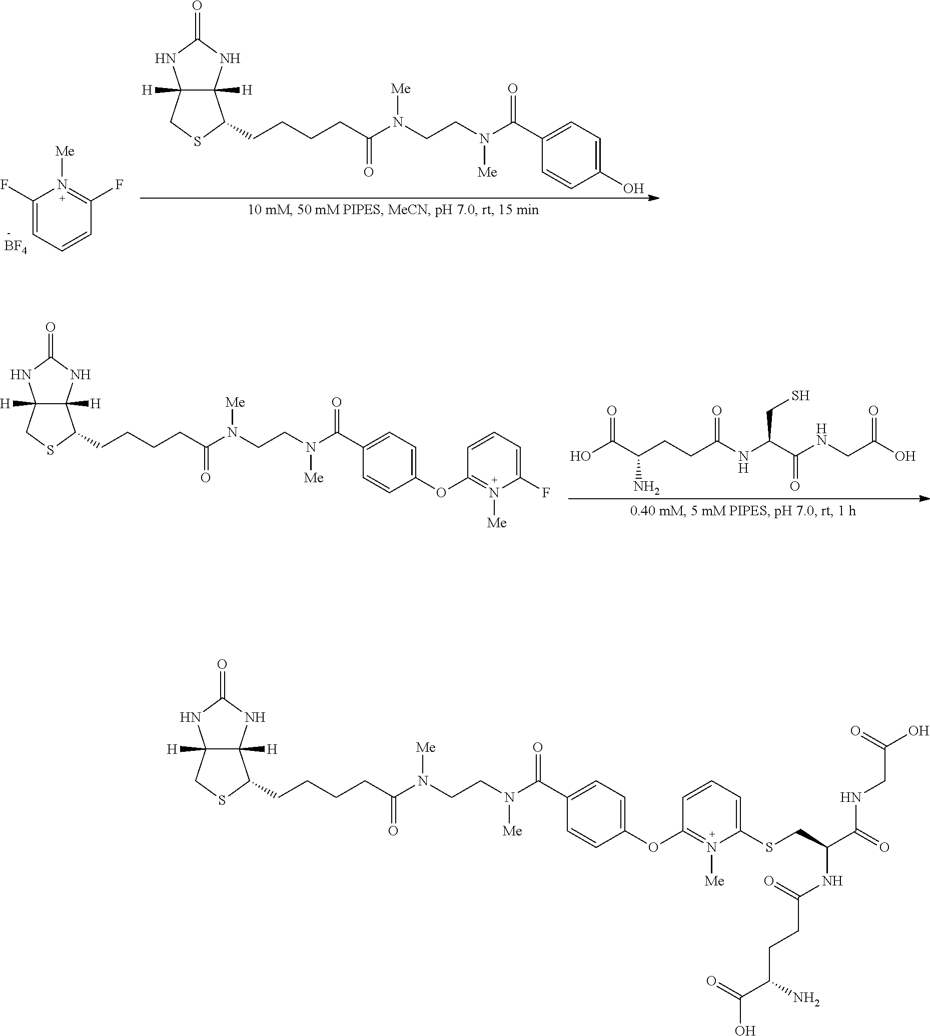

To a mixture of PIPES buffer (27.6 μL, pH 7) and biotin phenol derivative (8 μL, 50 mM in MeCN), an aliquot of CAP1-F (4.4 μL, 100 mM in MeCN) was added. This mixture was kept at room temperature for 15 min. An aliquot of this reaction mixture (16 μL) was added to a mixture of GSH (1.6 μL, 100 mM in H₂O), 50 mM PIPES buffer (40 μL, pH 7), and water (342 μL). The reaction mixture was purified by HPLC using solvent gradient 6 (Table 3, semi prep HPLC T_{R}=12.96 min) to obtain a white solid as a trifluoroacetic acid salt. Analytical HPLC using solvent gradient 11, T_{R}=12.38 min.

MS (ESI+): m/z calculated for [M+H]²⁺416.1, found 416.4. ESI-HRMS(+) m/z calculated for C₃₇H₅₂N₈O₁₀S²⁺ M⁺416.1619, found 416.1612. The structure of the product was also confirmed by tandem MS study.

### Example 35: Labeling GSH Using CAP2

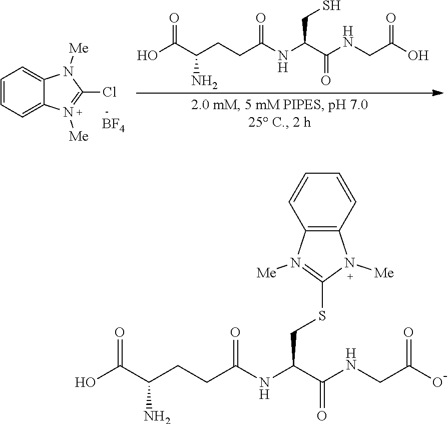

To a mixture of 50 mM PIPES buffer (40 μL, pH 7) and water (336 μL), an aliquot of GSH (8 μL, 100 mM in H₂O) was added. To this mixture, CAP2 (16 μL, 50 mM in H₂O) was added. The reaction mixture was purified by HPLC using solvent gradient 7 (Table 3, semi prep HPLC T_{R}=8.86 min) to obtain a white solid as a trifluoroacetic acid salt. Analytical HPLC using solvent gradient 11, T_{R}=6.22 min. to obtain a white solid as a trifluoroacetic acid salt.

MS (ESI+): m/z calculated for [M]⁺452.2, found 226.6. ESI-HRMS(+) m/z calculated for C₁₉H₂₆N₅O₆S⁺ M⁺452.1599, found 452.1603. The structure of the product was also confirmed by tandem MS study.

### Example 36: Labeling Reduced Oxytocin Using CAP1

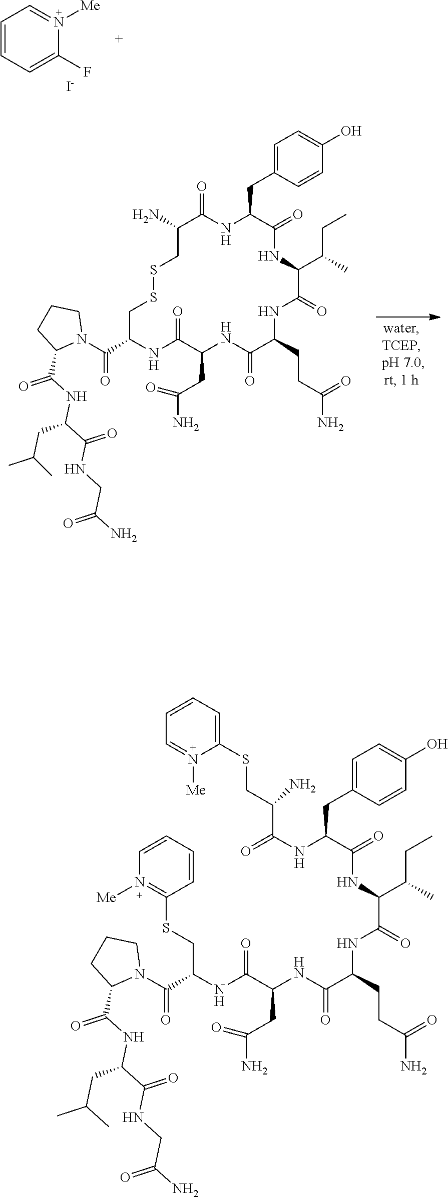

A stock solution of oxytocin (8 μL, 20 mM in water) was mixed with TCEP (4 μL, 0.5 M, pH 7) and then diluted with water (228 μL). After 5 min, an aliquot of CAP1 (160 μL, 5 mM) was added to the above mixture. The mixture was kept at room temperature for 1 h. The reaction mixture was purified by HPLC using solvent gradient 8 (Table 3, semi prep HPLC T_{R}=12.96 min) to obtain a white solid as a trifluoroacetic acid salt. Analytical HPLC using solvent gradient 11, T_{R}=12.03 min.

MS (ESI+): m/z calculated for M²+596.3, found 596.6. ESI-HRMS(+) m/z calculated for C₅₅H₈₀N₁₄O₁₂S₂²⁺ M²+596.2756, found 596.2714. The structure of the product was also confirmed by tandem MS study.

### Example 37: Labeling Reduced Oxytocin Using CAP10

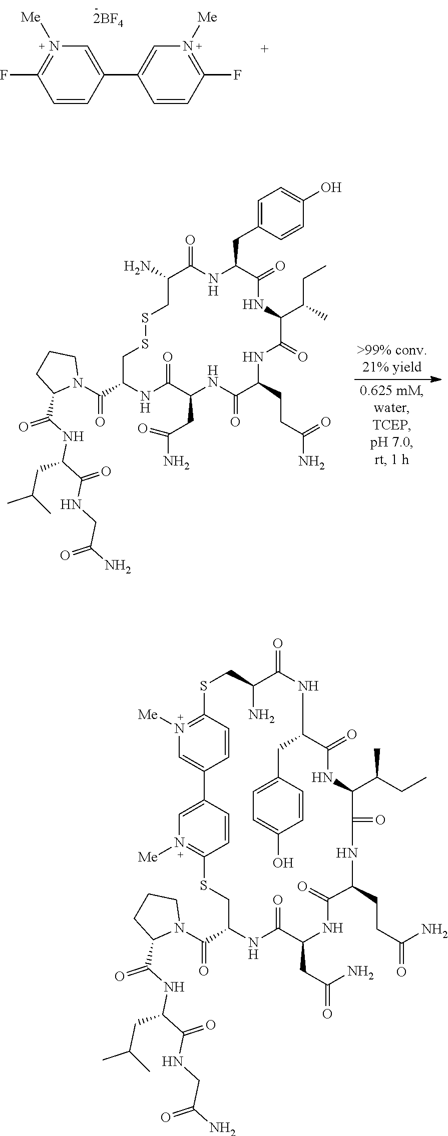

A stock solution of oxytocin (8 μL, 20 mM in water) was mixed with TCEP (4 μL, 0.5 M, pH 7) and then diluted with water (228 μL). After 5 min, an aliquot of CAP10 (16 μL, 10 mM) was added to the above mixture. The mixture was kept at room temperature for 1 h. The reaction mixture was purified by HPLC using solvent gradient 9 (Table 3, semi prep HPLC T_{R}=29.22 min) to obtain a white solid as a trifluoroacetic acid salt. Analytical HPLC using solvent gradient 11, T_{R}=13.75 min. MS (ESI+): m/z calculated for M²+595.2, found 595.8.

### Example 38: Labeling Cysteine Residues of Reduced Oxytocin Using CAP2

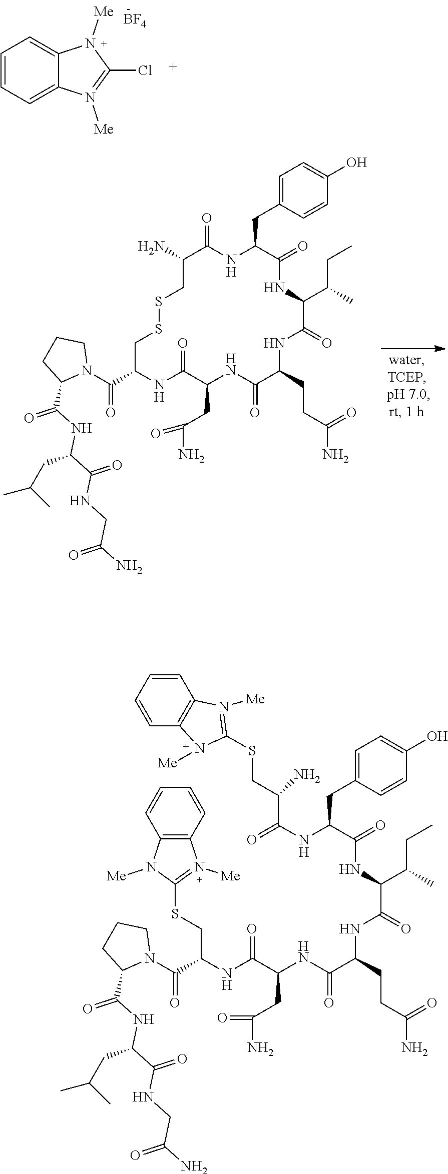

A stock solution of oxytocin (8 μL, 20 mM in water) was mixed with TCEP (4 μL, 0.5 M, pH 7) and then diluted with water (308 μL). After 5 min, an aliquot of CAP2 (80 μL, 5 mM) was added to the above mixture. The mixture was kept at room temperature for 1 h. The reaction mixture was purified by HPLC using solvent gradient 10 (Table 3, semi prep HPLC T_{R}=28.6 min) to obtain a white solid as a trifluoroacetic acid salt. Analytical HPLC using solvent gradient 11, T_{R}=13.43 min.

MS (ESI+): m/z calculated for M²+649.3, found 649.7. ESI-HRMS(+) m/z calculated for C₆₁H₈₆N₁₆O₁₂S₂²⁺ M²+649.3021, found 649.2945. The structure of this kinetic product was also confirmed by tandem MS study.

### Example 39: Labeling Cysteine Side Chains and the N-Terminus of Reduced Oxytocin Using CAP2

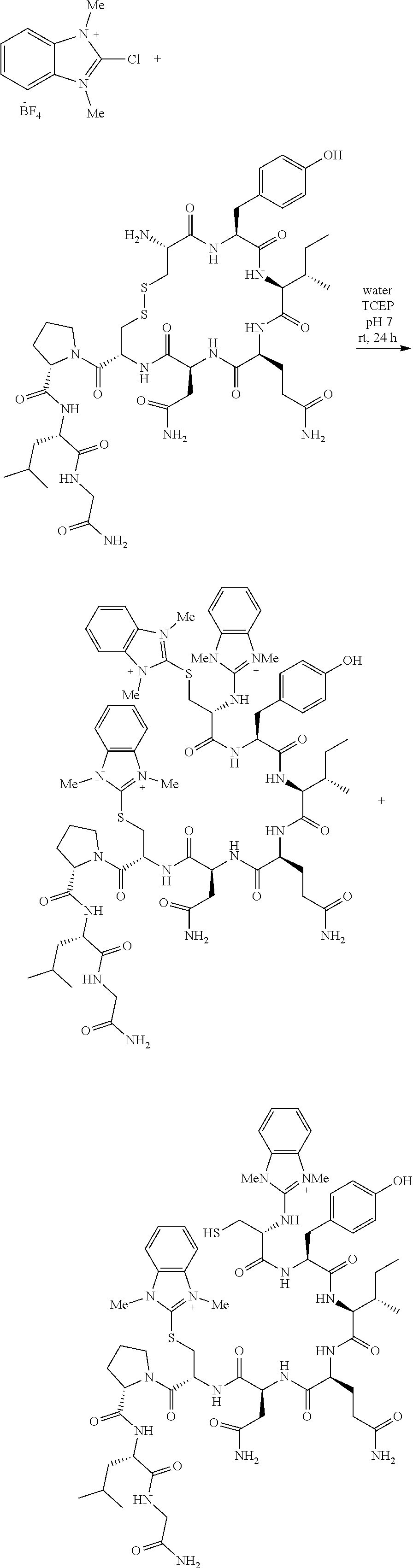

A stock solution of oxytocin (8 μL, 20 mM in water) was mixed with TCEP (4 μL, 0.5 M, pH 7) and then diluted with water (308 μL). After 5 min, an aliquot of CAP2 (80 μL, 5 mM) was added to the above mixture. The mixture was kept at room temperature for 24 h. The reaction mixture was purified by HPLC using solvent gradient 10 (Table 3). Two products were obtained as white solids. The product at T_{R}=38.4 min was functionalized by CAP2 on the N-terminus and Cys⁶ side chain. The structure of this thermodynamic product was also confirmed by tandem MS study. This product was converted from Cys¹,Cys⁶ doubly sulfur labeled oxytocin, which underwent the intramolecular CAP2 transfer from Cys¹ side chain to the N-terminus. Analytical HPLC using solvent gradient 11, T_{R}=15.07 min. MS (ESI+): m/z calculated for M²+649.3, found 649.7. The product at T_{R}=35.6 min was functionalized by CAP2 on Cys¹ side chain, Cys⁶ side chain, and the N-terminus. This product formed from the above the N-terminus, Cys⁶ doubly labeled oxytocin, the Cys¹ side chain of which further reacted with CAP2 in an intermolecular fashion. Analytical HPLC using solvent gradient 11, T_{R}=14.49 min.

MS (ESI+): m/z calculated for [M−H]²⁺721.3, found 721.8. ESI-HRMS(+) m/z calculated for C₆₁H₈₆N₁₆O₁₂S₂²⁺ M²+649.3021, found 649.2945. The structure of this thermodynamic product was also confirmed by tandem MS study.

### Example 40: Labeling the Cysteine Side Chain and the N-Terminus of Reduced H₂N—YC—CONH₂ Using CAP2

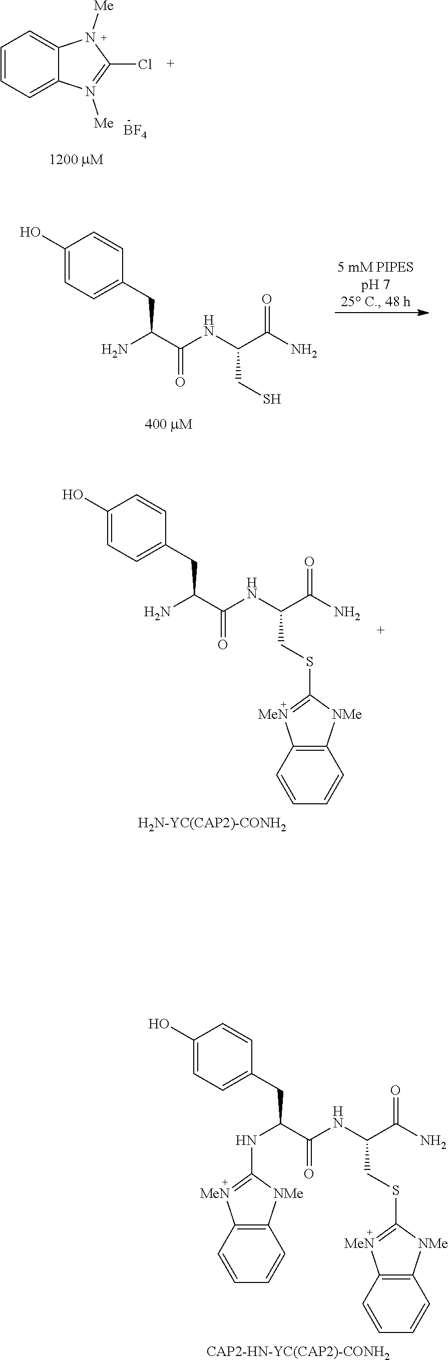

A stock solution of H₂N—YC—CONH₂ (4.6 μL, 35 mM in water) was diluted with water (346 μL) and PIPES buffer (50 mM in water, pH 7, 40 μL). An aliquot of CAP2 (9.6 μL, 50 mM) was added to the above mixture. The mixture was kept at room temperature for 48 h. The reaction mixture was purified by HPLC using solvent gradient 12 (Table 3). Two products were obtained as white solids. The product at T_{R}=11.4 min was functionalized by CAP2 on the Cys side chain. MS (ESI+): m/z calculated for [M−H]²⁺428.2, found 428.3. ESI-HRMS(+) m/z calculated for C₂₁H₂₆N₅O₃S⁺ M⁺428.1751, found 428.1717. The structure of this product was confirmed by tandem MS study. The doubly labeled product was functionalized by CAP2 on the Cys side chain and the N-terminus. Analytical HPLC using solvent gradient 11, T_{R}=14.7 min.

MS (ESI+): m/z calculated for [M−H]²⁺286.6, found 286.8. ESI-HRMS(+) m/z calculated for C₃₀H₃₄N₇O₃S⁺ M⁺286.6256, found 286.6244. The structure of this product was confirmed by tandem MS study.

### Example 41: Labeling the Cysteine Side Chain and the N-Terminus of Reduced H₂N—CY—CONH₂ Using CAP2

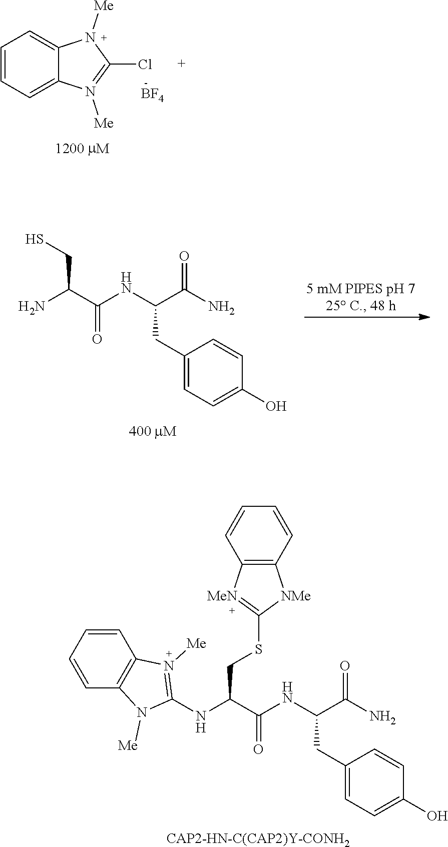

A stock solution of H₂N—CY—CONH₂ (8.4 μL, 19 mM in water) was diluted with water (342 μL) and PIPES buffer (50 mM in water, pH 7, 40 μL). An aliquot of CAP2 (9.6 μL, 50 mM) was added to the above mixture. The mixture was kept at room temperature for 48 h. The reaction mixture was purified by HPLC using solvent gradient 12 (Table 3). The doubly labeled product was functionalized by CAP2 on the Cys side chain and the N-terminus. Analytical HPLC using solvent gradient 12, T_{R}=14.2 min.

MS (ESI+): m/z calculated for [M−H]²⁺286.6, found 286.8. ESI-HRMS(+) m/z calculated for C₃₀H₃₄N₇O₃S⁺ M⁺286.6256, found 286.6246. The structure of this product was confirmed by tandem MS study.

### Example 42: Labeling Cysteine Side Chain and Tyrosine Side Chain of N-AcYPPPCNH₂ Using CAP2

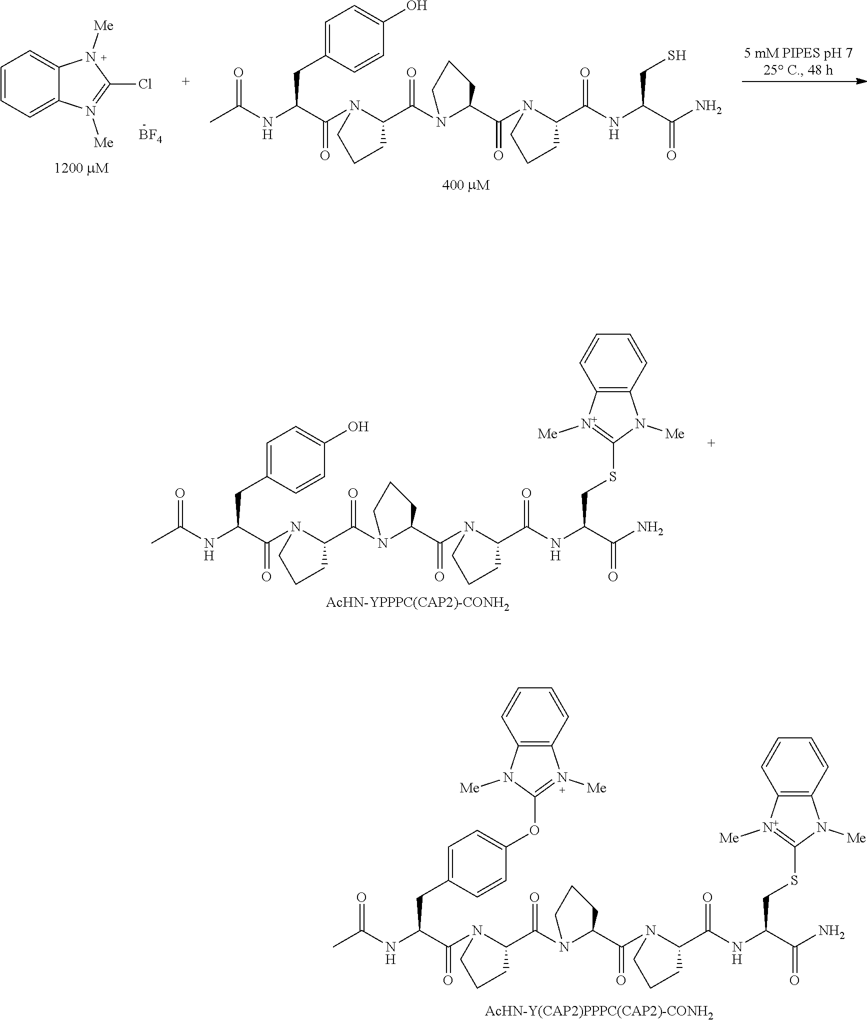

A stock solution of AcNH—YPPPC—CONH₂ (16 μL, 10 mM in water) was diluted with water (334 μL) and PIPES buffer (50 mM in water, pH 7, 40 μL). An aliquot of CAP2 (9.6 μL, 50 mM) was added to the above mixture. The mixture was kept at room temperature for 48 h. The reaction mixture was purified by HPLC using solvent gradient 13 (Table 3). Two products were obtained as white solids. The product at T_{R}=11.1 min was functionalized by CAP2 on the Cys side chain. MS (ESI+): m/z calculated for [M−H]²⁺761.3, found 761.5. ESI-HRMS(+) m/z calculated for C₃₈H₄₉N₈O₇S⁺ M⁺761.3440, found 761.3394. The structure of this product was confirmed by tandem MS study. The doubly labeled product was functionalized by CAP2 on the Cys side chain and the Tyr side chain. Analytical HPLC using solvent gradient 13, T_{R}=12.4 min.

MS (ESI+): m/z calculated for [M−H]²⁺453.2, found 453.5. ESI-HRMS(+) m/z calculated for C₄₇H₅₈N₁₀O₇S²⁺ M²⁺453.2100, found 453.2070. The structure of this product was confirmed by tandem MS study.

### Example 43: Labeling GSH with CAP3

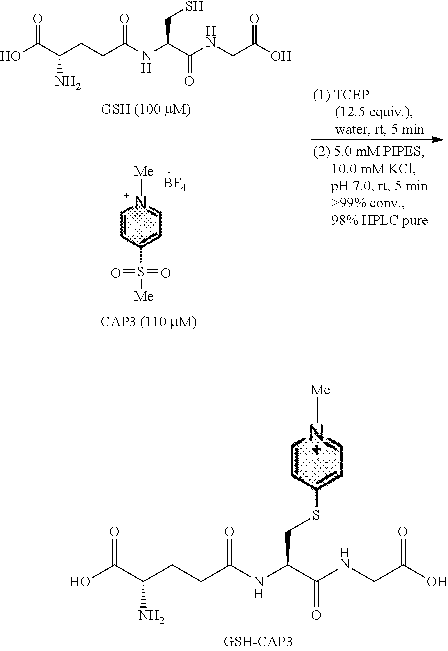

A stock solution of GSH (1.6 μL, 50 mM in water) was mixed with water (717 μL) and PIPES buffer (80 μL, pH 7, 50 mM PIPES, 100 mM KCl). An aliquot of CAP3 (1.76 μL, 50 mM in water) was added to this mixture. The reaction was kept at room temperature for 5 min. The crude product was purified by HPLC using solvent gradient 14 (Table 3, semi prep HPLC T_{R}=13.72 min, conversion 97.6%) to obtain a white solid as a trifluoroacetic acid salt. Analytical HPLC using solvent gradient 6, T_{R}=3.36 min.

MS (ESI+): m/z calculated for M⁺ 399.1, found 399.2. ESI-HRMS(+) m/z calculated for C₁₆H₂₃N₄O₆S⁺ M⁺399.1333, found 399.1325.

### Example 44: Labeling GSH with CAP4

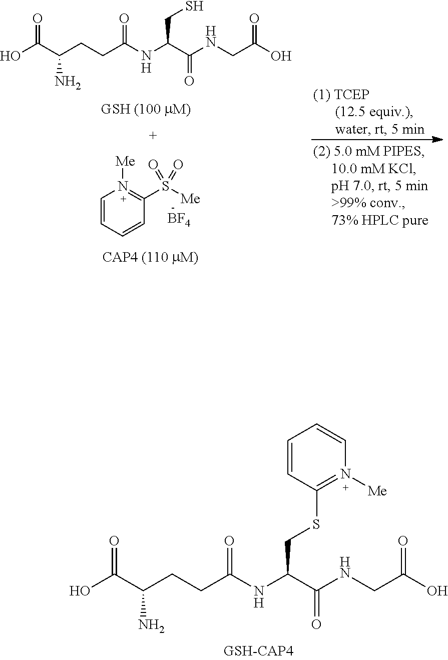

A stock solution of GSH (1.6 μL, 50 mM in water) was mixed with water (717 μL) and PIPES buffer (80 μL, pH 7, 50 mM PIPES, 100 mM KCl). An aliquot of CAP3 (1.76 μL, 50 mM in water) was added to this mixture. The reaction was kept at room temperature for 5 min. The crude product was purified by HPLC using solvent gradient 14 (Table 3, semi prep HPLC T_{R}=13.36 min, conversion 73.1%) to obtain a white solid as a trifluoroacetic acid salt. Analytical HPLC using solvent gradient 6, T_{R}=3.35 min. ESI-HRMS(+) m/z calculated for C₁₆H₂₃N₄O₆S⁺ M⁺399.1333, found 399.1345.

### Example 45: Labeling Reduced Somatostatin with CAP3

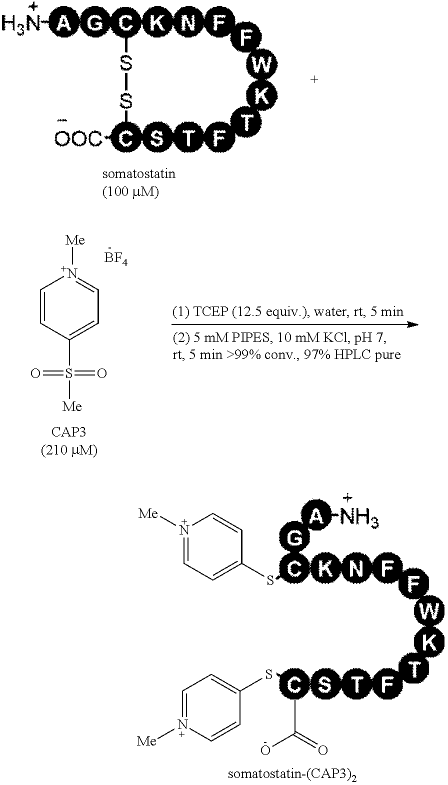

A stock solution of somatostatin (4.59 μL, 4.36 mM in water) was mixed with TCEP (0.5 μL, 0.5 M, pH 7). After 5 min, the mixture was diluted with water (173 μL) and PIPES buffer (20 μL, pH 7, 50 mM PIPES, 100 mM KCl). An aliquot of CAP4 (0.84 μL, 50 mM in water) was added to this mixture. The reaction was kept at room temperature for 5 min. The crude product was purified by HPLC using solvent gradient 15 (Table 3, semi prep HPLC T_{R}=10.69 min, conversion 96.5%) to obtain a white solid as a trifluoroacetic acid salt. Analytical HPLC using solvent gradient 6, T_{R}=14.29 min.

MS (ESI+): m/z calculated for [M+H]³⁺608.3, found 608.5. ESI-HRMS(+) m/z calculated for C₈₈H₁₁₈N₂₀O₁₉S₂²⁺ M²⁺911.4157, found 911.4141.

### Example 46: Labeling Reduced Somatostatin with CAP4

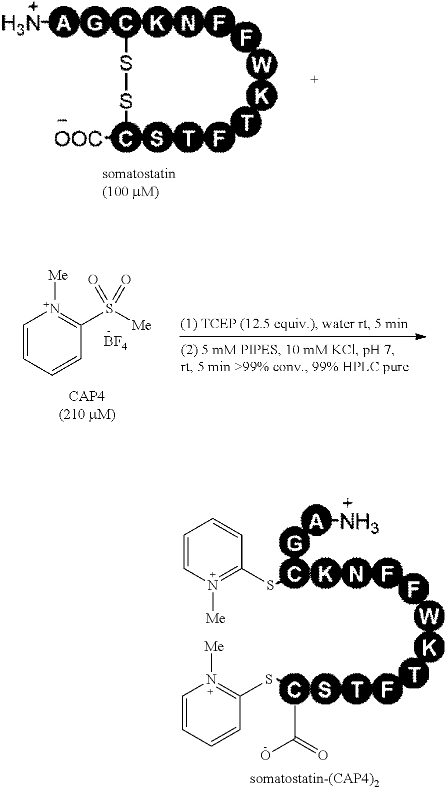

A stock solution of somatostatin (5.62 μL, 3.56 mM in water) was mixed with TCEP (0.5 μL, 0.5 M, pH 7). After 5 min, the mixture was diluted with water (173 μL) and PIPES buffer (20 μL, pH 7, 50 mM PIPES, 100 mM KCl). An aliquot of CAP4 (0.84 μL, 50 mM in water) was added to this mixture. The reaction was kept at room temperature for 5 min. The crude product was purified by HPLC using solvent gradient 15 (Table 3, semi prep HPLC T_{R}=10.74 min, conversion 100%) to obtain a white solid as a trifluoroacetic acid salt. Analytical HPLC using solvent gradient 6, T_{R}=14.35 min.

ESI-HRMS(+) m/z calculated for C₈₈H₁₁₈N₂₀O₁₉S₂²⁺ M²⁺911.4157, found 911.4137.

### Example 47: Labeling Reduced Oxytocin with CAP3

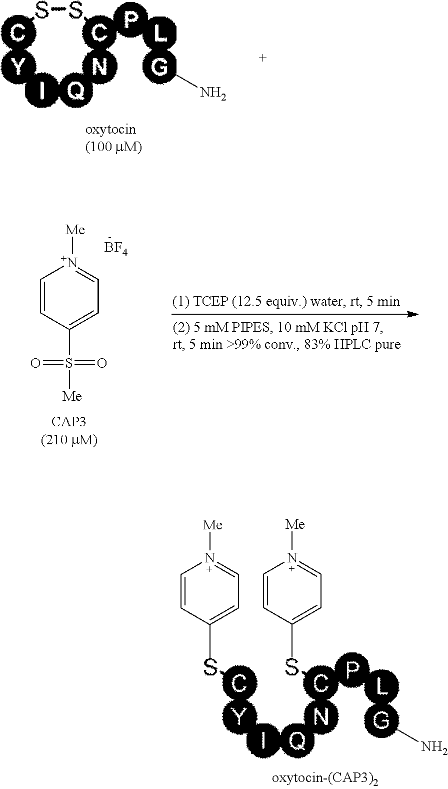

A stock solution of oxytocin (4 μL, 20 mM in water) was mixed with TCEP (2 μL, 0.5 M, pH 7). After 5 min, the mixture was diluted with water (711 μL) and PIPES buffer (80 μL, pH 7, 50 mM PIPES, 100 mM KCl). An aliquot of CAP3 (3.36 μL, 50 mM in water) was added to this mixture. The reaction was kept at room temperature for 5 min. The crude product was purified by HPLC using solvent gradient 10 (Table 3, semi prep HPLC T_{R}=23.14 min, conversion 82.6%) to obtain a white solid as a trifluoroacetic acid salt. Analytical HPLC using solvent gradient 6, T_{R}=12.10 min.

MS (ESI+): m/z calculated for M²⁺596.3, found 596.8. ESI-HRMS(+) m/z calculated for C₅₅H₈₀N₁₄O₁₂S₂²⁺ M²⁺596.2756, found 596.2739.

### Example 48: Labeling Reduced Oxytocin with CAP4

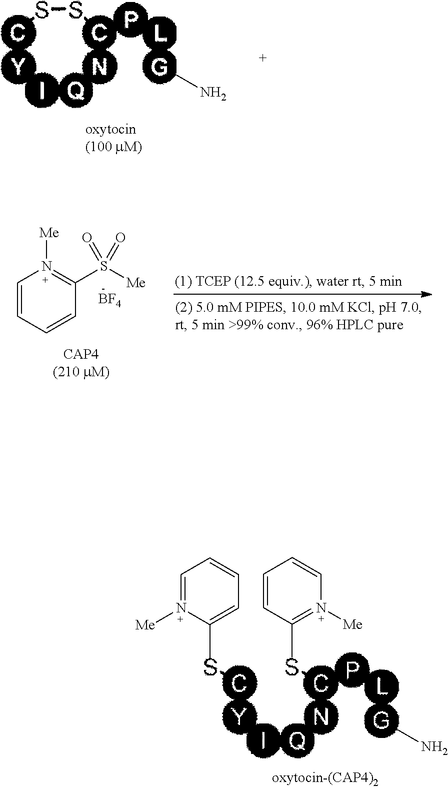

A stock solution of oxytocin (4 μL, 20 mM in water) was mixed with TCEP (2 μL, 0.5 M, pH 7). After 5 min, the mixture was diluted with water (711 μL) and PIPES buffer (80 μL, pH 7, 50 mM PIPES, 100 mM KCl). An aliquot of CAP4 (3.36 μL, 50 mM in water) was added to this mixture. The reaction was kept at room temperature for 5 min. The crude product was purified by HPLC using solvent gradient 10 (Table 3, semi prep HPLC T_{R}=22.89 min, conversion 95.7%) to obtain white solid oxytocin-(CAP4)₂ as trifluoroacetic acid salt. Analytical HPLC using solvent gradient 6, T_{R}=12.15 min.

ESI-HRMS(+) m/z calculated for C₅₅H₈₀N₁₄O₁₂S₂²⁺ M²⁺596.2756, found 596.2718.

### Example 49: Stapling Reduced Somatostatin with CAP5

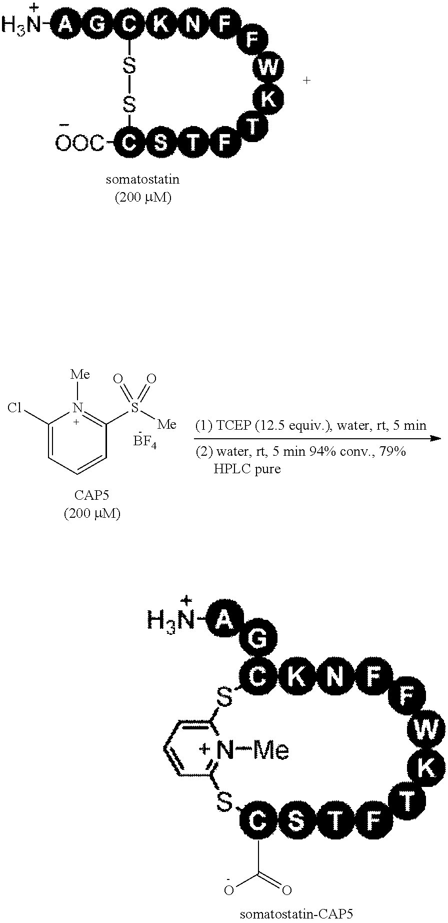

A stock solution of somatostatin (11.24 μL, 3.56 mM in water) was mixed with TCEP (1 μL, 0.5 M, pH 7). After 5 min, the mixture was diluted with water (183.8 μL). An aliquot of freshly prepared CAP5 in MeCN (4 μL, 10 mM) was added to this mixture. The reaction was kept at room temperature for 5 min. The crude product was purified by HPLC using solvent gradient 16 (Table 3, semi prep HPLC T_{R}=12.63 min, conversion 78.6%) to obtain white solid somatostatin-CAP5 as a trifluoroacetic acid salt. Analytical HPLC using solvent gradient 6, T_{R}=14.68 min.

MS (ESI+): m/z calculated for [M+H]²⁺864.9, found 865.6. ESI-HRMS(+) m/z calculated for C₈₂H₁₁₁N₁₉O₁₉S₂²⁺[M+H]²⁺864.8867, found 864.8845.

Results obtained under different conditions are summarized in Table 4 below.

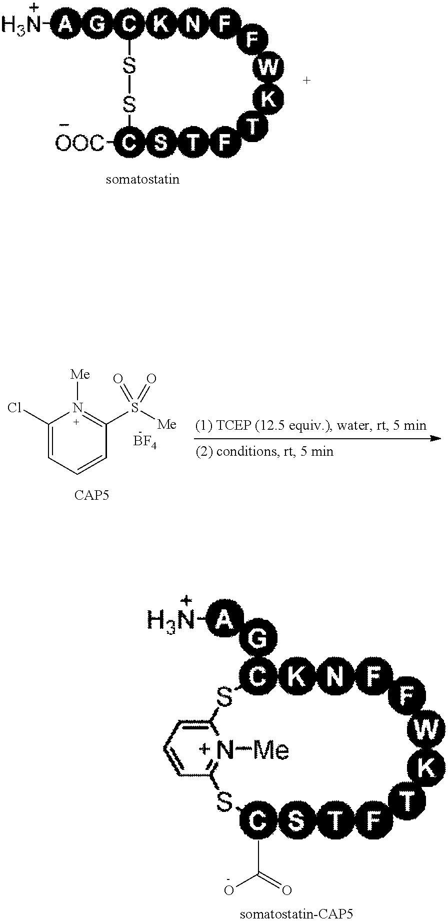

TABLE 4

| [Somatostatin] | [CAP5] | | Conv. | Purity |
|---|---|---|---|---|
| (μM) | (μM) | media | (%)^{a} | (%)^{a} |
| 200 | 200 | Water, 25° C. | 94 | 79 |
| 100 | 100 | water, 25° C. | 93 | 82 |
| 200 | 200 | 5 mM PIPES, 10 mM KCl, | 96 | 84 |
| | | pH 7, 25° C. | | |
| 100 | 100 | 5 mM PIPES, 10 mM KCl, | 96 | 87 |
| | | pH 7, 25° C. | | |

^{a}Based on HPLC chromatogram integration at 250 nm.

### Example 50: Stapling Reduced Somatostatin with CAP6

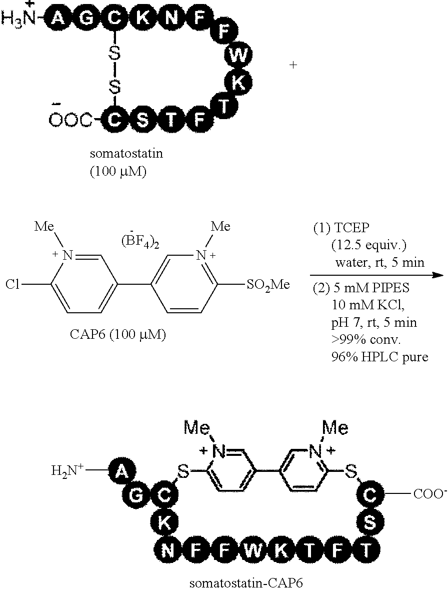

A stock solution of somatostatin (5.62 μL, 3.56 mM in water) was mixed with TCEP (0.5 μL, 0.5 M, pH 7). After 5 min, the mixture was diluted with water (171.9 μL) and PIPES buffer (20 μL, pH 7, 50 mM PIPES, 100 mM KCl). An aliquot of freshly prepared CAP6 in MeCN (2 μL, 10 mM) was added to this mixture. The reaction was kept at room temperature for 5 min. The crude product was purified by HPLC using solvent gradient 16 (Table 3, semi prep HPLC T_{R}=10.35 min) to obtain a white solid as a trifluoroacetic acid salt. Analytical HPLC using solvent gradient 6, T_{R}=14.19 min.

MS (ESI+): m/z calculated for [M+H]³⁺607.6, found 607.8. ESI-HRMS(+) m/z calculated for C₈₈H₁₁₇N₂₀O₁₉S₂³⁺[M+H]³⁺607.2743, found 607.2757.

Results obtained under different conditions are summarized in Table 5 below.

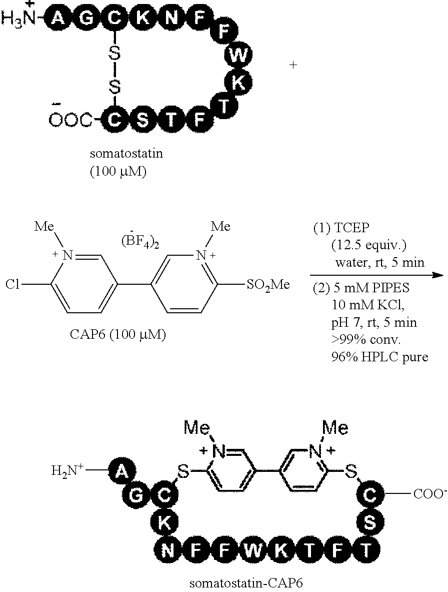

TABLE 5

| [Somatostatin] | [CAP6] | | Conv. | Purity |
|---|---|---|---|---|
| (μM) | (μM) | media | (%)^{a} | (%)^{a} |
| 100 | 100 | 5 mM PIPES, 10 mM KCl, | >99 | 96 |
| | | pH 7, 25° C. | | |
| 100 | 100 | water, 25° C. | >99 | 83 |
| 50 | 50 | 5 mM PIPES, 10 mM KCl, | >99 | 87 |
| | | pH 7, 25° C. | | |
| 25 | 25 | 5 mM PIPES, 10 mM KCl, | >99 | 92 |
| | | pH 7, 25° C. | | |

^{a}Based on HPLC chromatogram integration at 250 nm.

### Example 51: Stapling Reduced Somatostatin with CAP7

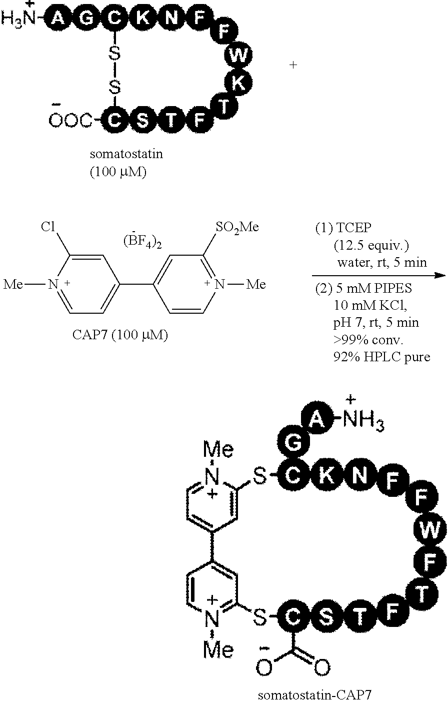

A stock solution of somatostatin (5.62 μL, 3.56 mM in water) was mixed with TCEP (0.5 μL, 0.5 M, pH 7). After 5 min, the mixture was diluted with water (171.9 μL) and PIPES buffer (20 μL, pH 7, 50 mM PIPES, 100 mM KCl). An aliquot of freshly prepared CAP7 in MeCN (2 μL, 10 mM) was added to this mixture. The reaction was kept at room temperature for 5 min. The crude product was purified by HPLC using solvent gradient 16 (Table 3, semi prep HPLC T_{R}=11 min, conversion 91.5%) to obtain white solid somatostatin-CAP7 as a trifluoroacetic acid salt.

Analytical HPLC using solvent gradient 6, T_{R}=14.55 min.

ESI-HRMS(+) m/z calculated for C₈₈H₁₁₇N₂₀O₁₉S₂³⁺[M+H]³⁺607.2743, found 607.2780.

### Example 52: Stapling Reduced Somatostatin with CAP8

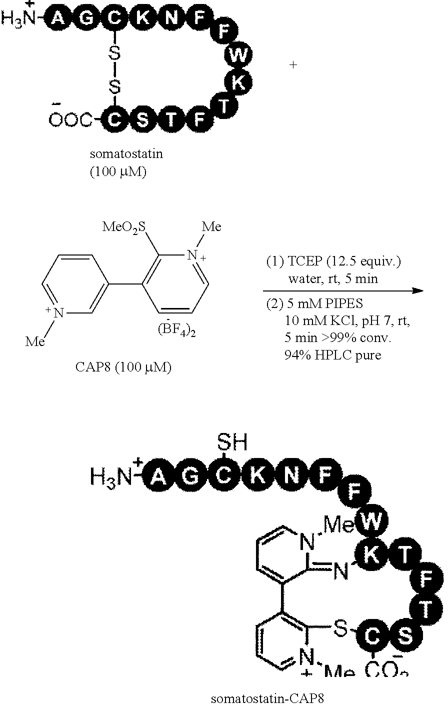

A stock solution of somatostatin (5.62 μL, 3.56 mM in water) was mixed with TCEP (0.5 μL, 0.5 M, pH 7). After 5 min, the mixture was diluted with water (171.9 μL) and PIPES buffer (20 μL, pH 7, 50 mM PIPES, 100 mM KCl). An aliquot of freshly prepared CAP8 in MeCN (2 μL, 10 mM) was added to this mixture. The reaction was kept at room temperature for 5 min. The crude product was purified by HPLC using solvent gradient 16 (Table 3, semi prep HPLC T_{R}=11.1 min, conversion 93.6%) to obtain white solid somatostatin-CAP8 as a trifluoroacetic acid salt.

Analytical HPLC using solvent gradient 6, T_{R}=14.66 min. ESI-HRMS(+) m/z calculated for C₈₈H₁₁₇N₂₀O₁₉S₂³⁺[M+H]³⁺607.2743, found 607.2742.

### Example 53: Stapling Reduced Oxytocin with CAP5

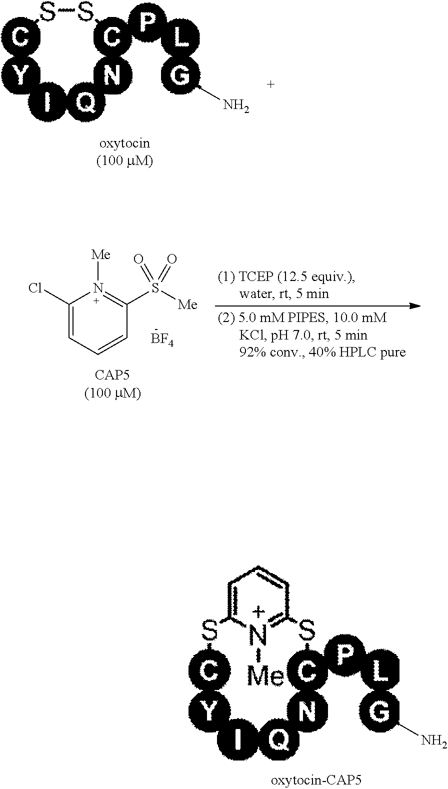

A stock solution of oxytocin (4 μL, 20 mM in water) was mixed with TCEP (2 μL, 0.5 M, pH 7). After 5 min, the mixture was diluted with water (706 μL) and PIPES buffer (80 μL, pH 7, 50 mM PIPES, 100 mM KCl). An aliquot of freshly prepared CAP5 in MeCN (8 μL, 10 mM) was added to this mixture. The reaction was kept at room temperature for 5 min. The crude product was purified by HPLC using solvent gradient 10 (Table 3, semi prep HPLC T_{R}=26.26 min, conversion 43.6%) to obtain white solid oxytocin-CAP5 as a trifluoroacetic acid salt. Analytical HPLC using solvent gradient 6, T_{R}=12.91 min.

MS (ESI+): m/z calculated for [M+H]²⁺1098.5, found 1098.7. ESI-HRMS(+) m/z calculated for C₄₉H₇₂N₁₃O₁₂S₂⁺[M+H]⁺ 1098.4860, found 1098.4872.

### Example 54: Stapling Reduced Oxytocin with CAP6

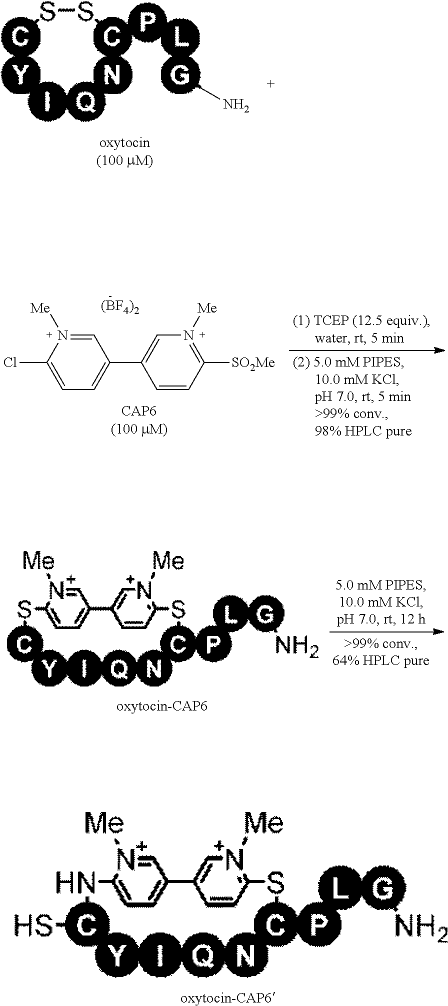

A stock solution of oxytocin (4 μL, 20 mM in water) was mixed with TCEP (2 μL, 0.5 M, pH 7). After 5 min, the mixture was diluted with water (706 μL) and PIPES buffer (80 μL, pH 7, 50 mM PIPES, 100 mM KCl). An aliquot of freshly prepared CAP6 in MeCN (8 μL, 10 mM) was added to this mixture. The reaction was kept at room temperature for 5 min. The crude product was purified by HPLC using solvent gradient 10 (Table 3, semi prep HPLC T_{R}=22.98 min, conversion 97.6%) to obtain white solid oxytocin-CAP6 as a trifluoroacetic acid salt. Analytical HPLC using solvent gradient 6, T_{R}=12.09 min.

MS (ESI+): m/z calculated for M²+595.2, found 595.8. ESI-HRMS(+) m/z calculated for C₅₅H₇₈N₁₄O₁₂S₂²⁺ M²⁺595.2677, found 595.2674. The obtained cysteine-arylated product oxytocin-CAP6 isomerized through intramolecular S→N-terminus transfer after storing in pH 7 PIPES buffer at room temperature for 12 h. (Table 3, solvent gradient 3, semi prep HPLC T_{R}=30.25 min, conversion 64%) to obtain white solid oxytocin-CAP6 isomer as a trifluoroacetic acid salt. Analytical HPLC using solvent gradient 6, T_{R}=13.69 min.

MS (ESI+): m/z calculated for M²+595.2, found 595.8. ESI-HRMS(+) m/z calculated for C₅₅H₇₈N₁₄O₁₂S₂²⁺ M²⁺595.2677, found 595.2686.

### Example 55: One-Pot Two-Step Labeling of GSH with CAP9 and Phenol

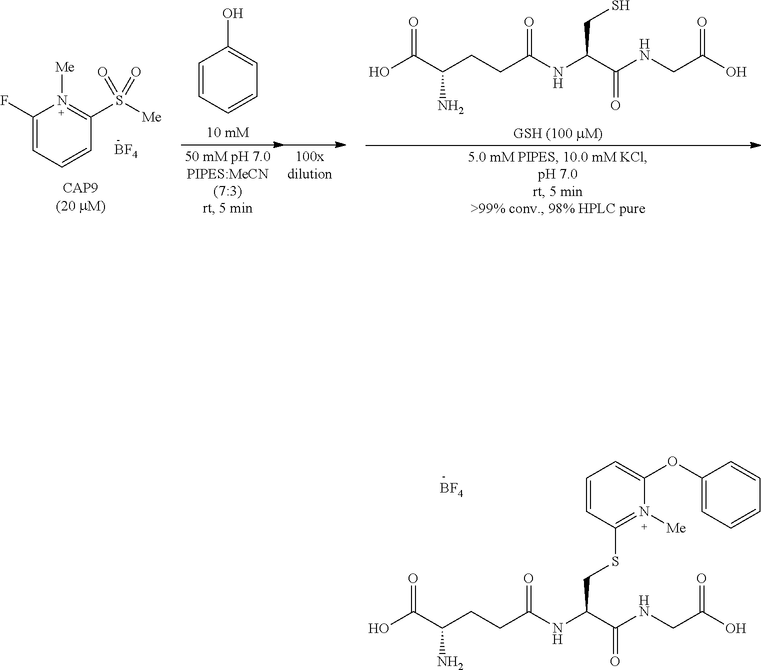

To a mixture of PIPES buffer (14 μL, pH 7) and PhOH (2 μL, 100 mM in MeCN), an aliquot of CAP9 (4 μL, 100 mM in MeCN) was added. The reaction was kept at room temperature for 5 min. An aliquot of this solution (4 μL) was added to a mixture of GSH (4 μL, 10 mM in water), PIPES buffer (40 μL, pH 7, 50 mM PIPES, 100 mM KCl), and water (352 μL). This reaction was kept at room temperature for 5 min. The crude product was purified by HPLC using solvent gradient 17 (Table 3, semi prep HPLC T_{R}=13.67 min) to obtain a white solid as a trifluoroacetic acid salt.

MS (ESI+): m/z calculated for M⁺ 491.2, found 491.2. ESI-HRMS(+) m/z calculated for C₂₂H₂₇N₄O₇S⁺ M⁺491.1595, found 491.1586.

### Example 56: Labeling Papain with CAP1 in PIPES Buffer and the Subsequent Kinetic Study

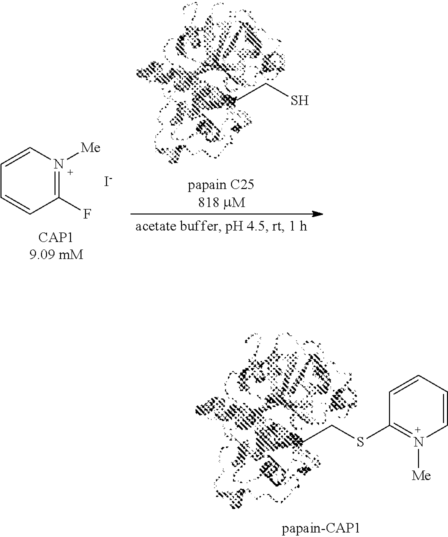

An aliquot of papain (9 μL, 1 mM suspension in 50 mM sodium acetate, pH 4.5, containing 0.01% thymol) was mixed with 50 mM CAP1 (2 μL, in water). This solution was kept at room temperature for 40 min. A portion of this solution (9 μL) was added to a cuvette containing Nu-benzoyl-DL-arginine 4-nitroanilide hydrochloride (BAPNA, 3 mM in DMSO, 491 μL) and 20 mM PIPES buffer (2500 μL, pH 6.27) with 2 mM EDTA and 300 mM NaCl. The progress of BAPNA hydrolysis was monitored at 400 nm by UV-Vis spectroscopy with stirring at 25° C.

The control experiment was conducted under similar conditions. An aliquot of papain (9 μL, 1 mM suspension in 50 mM sodium acetate, pH 4.5, containing 0.01% thymol) was mixed with a 20 mM PIPES buffer (2 μL, pH 6.27) containing 2 mM EDTA and 300 mM NaCl. This solution was kept at room temperature for 40 min. A portion of this solution (9 μL) was added to a cuvette containing BAPNA (3 mM in DMSO, 491 μL) and 20 mM PIPES buffer (2500 μL, pH 6.27) with 2 mM EDTA and 300 mM NaCl. The progress of BAPNA hydrolysis was monitored at 400 nm by UV-Vis spectroscopy with stirring at 25° C., which revealed inhibited enzymatic activity of papain, in turn, suggesting the arylation of cysteine at the active site.

### Example 57: Labeling BSA with CAP1 in PIPES Buffer

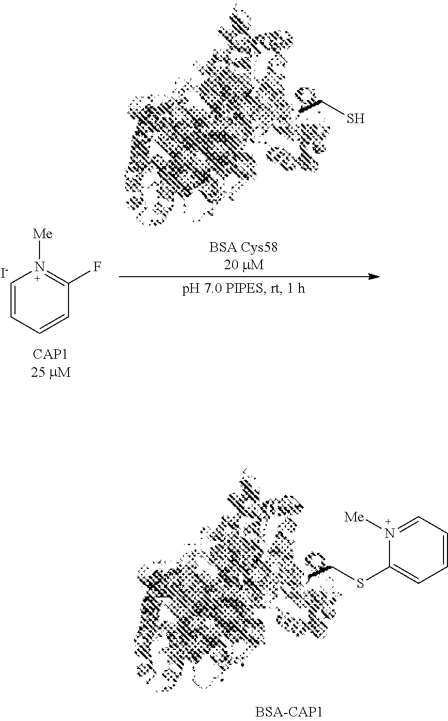

The stock solution of bovine serum albumin (BSA) in 0.9% saline solution with 0.05% sodium azide (2 mg/mL, 30 μM, 20 μL) was mixed with pH 7 PIPES (8.5 μL). An aliquot of CAP1 in water (0.5 mM, 1.5 μL, 1.25 equiv.) was added to this mixture. The reaction was kept at room temperature for 1 h to generate a solution of BSA-CAP1.

This sample was digested using Trypsin Singles, Proteomic Grade Kit (Sigma-Aldrich, Catalog Number: T7575) according to the manufacturer's directions. Briefly, trypsin solubilizing reagent (1 μL) was added to a trypsin singles vial. For native protein digestion, BSA (2 mg/mL, 30 μM, 20 μL) and tris buffer (pH 10.4, 20 μL) were combined in a microcentrifuge tube and transferred to the trypsin singles vial.

For arylated protein digestion, the sample from the previous step was added to the respective trypsin singles vial. In both cases, trypsin reaction buffer (49 μL, 40 mM NH₄HCO₃ aq. and 9% v/v CH₃CN) was transferred to each vial. The digestion mixture was vortexed and centrifuged before a 5 h incubation at 37° C. The samples were acidified with 10% v/v CF₃CO₂H aq. (1.6 μL). The samples were diluted 1:10 with CH₃CN and analyzed by MALDI-TOF spectrometry with u-cyano-4-hydroxycinnamic acid (CHCA) matrix. Mass data were compared to mass lists generated from Expasy PeptideMass database (ID: ALBU_BOV) and indicated the formation of desired BSA-CAP1.

### Example 58: Labeling GFP V150C with CAP1-CCH in PIPES and Phosphate Buffer

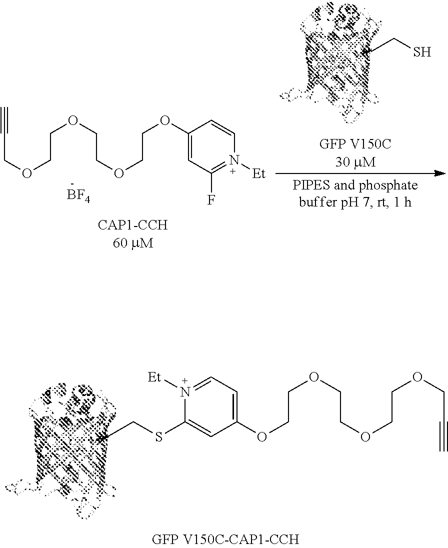

The stock solution of GFP V150C (10 μL, 60 μM in 50 mM phosphate buffer) was mixed with pH 7 PIPES (8.8 μL). An aliquot of CAP1-CCH in water (1 mM, 1.2 μL, 2 equiv.) was added to this mixture. The reaction was kept at room temperature for 1 h. The resulting mixture was analyzed with MALDI-MS using super-DHB matrix (Supelco). MS (MALDI+): m/z calculated for M+30120, found 30126.

### Example 59

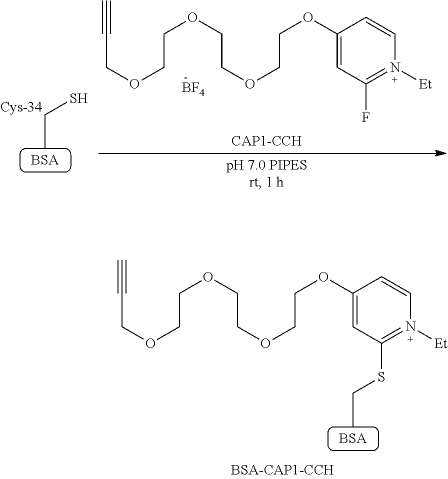

A stock solution of BSA (2 mg/L, 30 μM, in 0.9% NaCl saline with NaN₃ inhibitor, 10 μL) was diluted with PIPES buffer (50 mM, pH 7, 8.8 μL). An aliquot of CAP1-CCH (1.2 μL, 1 mM in water, 4 equiv.) was added to the above mixture. The mixture was kept at room temperature for 1 h before MALDI analysis using sinapinic acid matrix.

MS (MALDI+): m/z calculated for [BSA-CAP1-CCH]⁺66755, found 66755; m/z calculated for [BSA-CAP1-CCH]²⁺33378, found 33378.

### Example 60: Labeling Bovine Serum Albumin (BSA) with CAP1-CCH and 7-Azido-Methylcoumarin in PIPES Buffer

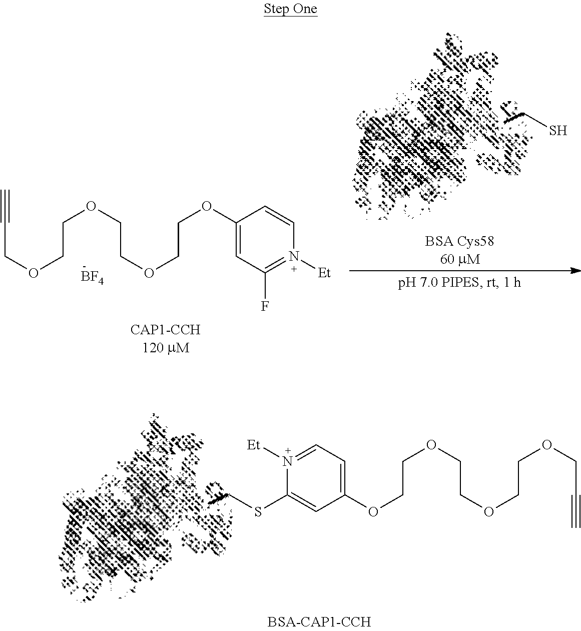

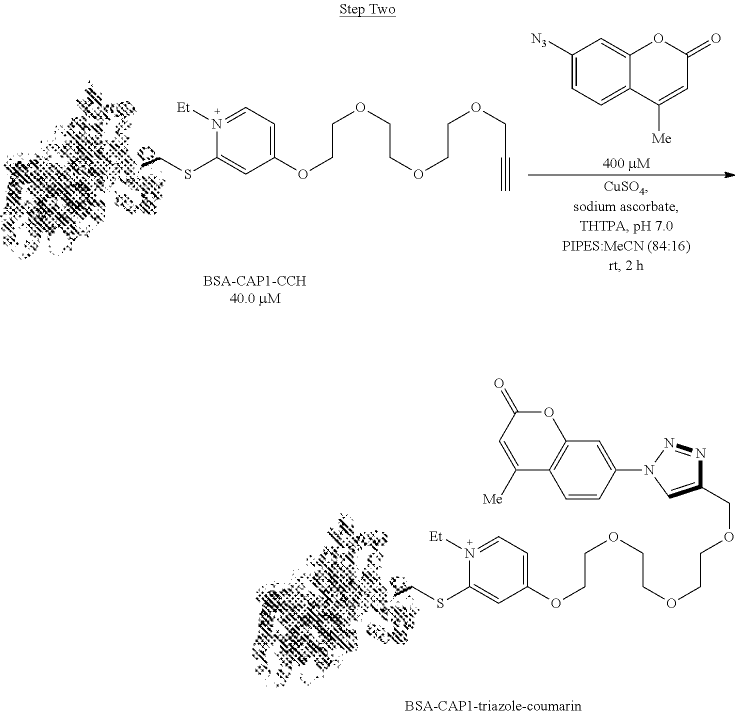

A stock solution of bovine serum albumin (BSA) was freshly prepared by dissolving lyophilized material (Fisher Scientific, catalog number BP671) in water. This solution (11.2 mg/mL, 169 μM, 3.56 μL) was mixed with pH 7 PIPES (5.24 μL). An aliquot of CAP1-CCH in water (1 mM, 1.2 μL, 2 equiv.) was added to this mixture. The reaction was kept at room temperature for 1 h to generate BSA-CAP1-CCH solution (60 μM). In a separate microcentrifuge tube, the catalyst solution was prepared by mixing CuSO₄ (80 mM, 1.25 μL, in water) with tris(3-hydroxypropyltriazolylmethyl)amine (THTPA, 50 mM, 10 μL, in water) and sodium ascorbate (100 mM, 6 μL, in water). After 5 min, this solution was diluted with pH 8 PBS (22.8 μL). Sodium ascorbate aq. (10 mM, 0.06 μL) was added to BSA-CAP1-CCH solution (60 μM, 6.7 μL), followed by the addition of the catalyst solution (1.6 μL), 7-azido-methylcoumarin in MeCN (2.5 mM, 1.6 μL, 10 equiv. relative to BSA-CAP1-CCH), and water (0.04 μL). The reaction was kept at room temperature for 2 h in the dark.

This sample was digested using Trypsin Singles, Proteomic Grade Kit (Sigma-Aldrich, Catalog Number: T7575) according to the manufacturer's directions. Briefly, trypsin solubilizing reagent (1 μL) was added to a trypsin singles vial. For native protein digestion, BSA (11.2 mg/mL, 169 μM, 3.56 μL), PIPES (pH 7, 5.24 μL), and water (1.2 μL) were combined in a microcentrifuge tube. An aliquot of this solution (6.7 μL) was mixed with water (3.3 μL) and transferred to the trypsin singles vial. This mixture was further diluted with water (20.1 μL). For arylated proteins, the sample from the previous step was mixed with water (20.1 μL) added to the respective trypsin singles vial. In both cases, trypsin reaction buffer (30.2 μL, 40 mM NH₄HCO₃ aq. and 9% v/v MeCN) was transferred to each vial. The digestion mixture was vortexed and centrifuged before a 5 h incubation at 37° C. The samples were diluted 1:10 with MeCN containing 0.1 v % CF₃CO₂H and analyzed by MALDI-TOF spectrometry with u-cyano-4-hydroxycinnamic acid (CHCA) matrix. Mass data were compared to mass lists generated from Expasy PeptideMass database (ID: ALBU_BOV). A similar result was obtained using

BSA:CAP1-CCH:7-azido-methylcoumarin:CuSO₄ in 1:10:50:10 ratio.

### Example 61: One-Pot Two-Step Labeling of GFP V150C with CAP1-F and Tetramethylrhodamine Phenol Derivative

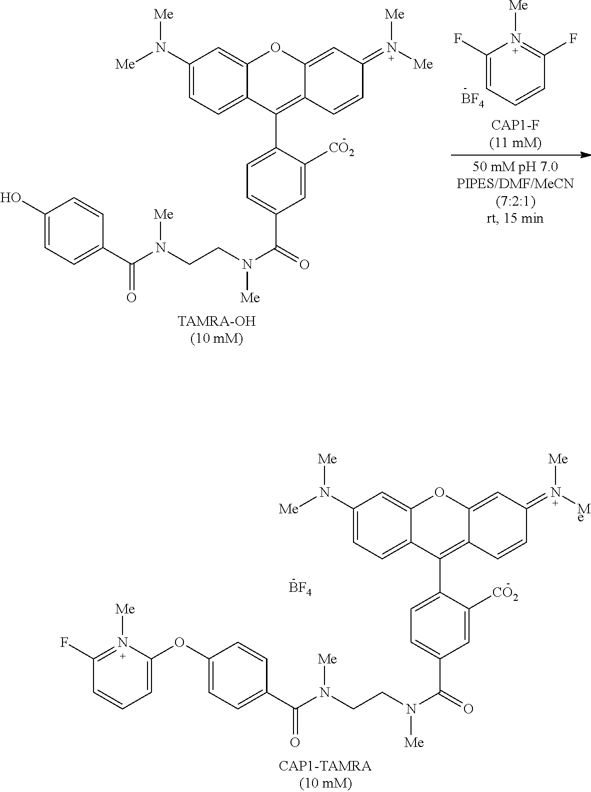

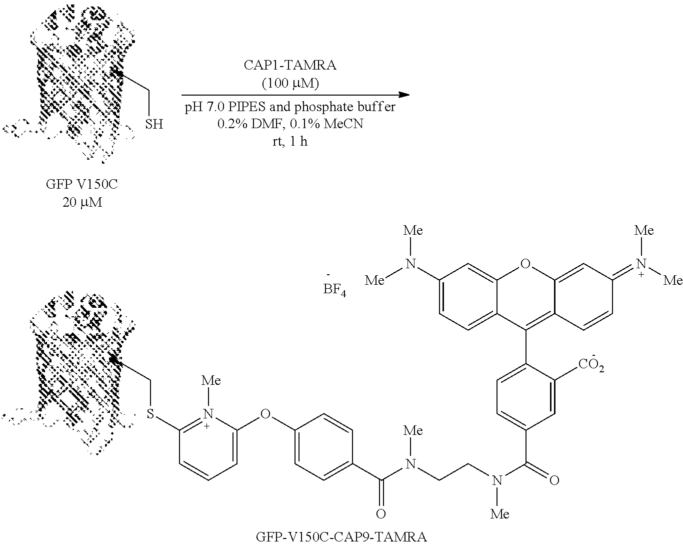

An aliquot of CAP1-F (2.2 μL, 100 mM in MeCN) was added to a mixture of PIPES buffer (13.5 μL, pH 7) and tetramethylrhodamine phenol derivative (4.26 μL, 47 mM in DMF). The reaction was kept at room temperature for 15 min. An aliquot of this solution (4 μL, 10 mM) was diluted with water (16 μL) to give a 2 mM CAP1-TAMRA solution. An aliquot of the CAP1-TAMRA solution (1 μL, 2 mM, 2 equiv.) was mixed with GFP V150C (16.6 μL, 60 μM in 50 mM phosphate buffer) in pH 7 PIPES (2.4 μL). The reaction mixture was analyzed by gel electrophoresis. Protein samples were loaded in 2× Laemmli sample buffer (Bio-Rad 1610737) and resolved by SDS-PAGE (Bio-Rad, Any kD™ mini-PROTEAN® TGX Stain-Free™ protein gels, 4568126). The gel was visualized with an Azure Sapphire Biomolecular Imager for GFP (excitation 488 nm; emission 518 nm) and TAMRA (excitation 520 nm; emission 565 nm), which confirmed the formation of the desired GFP-V150C-CAP1-TAMRA conjugate. Analogous results were obtained with 4 equiv. of CAP1-TAMRA under similar reaction conditions.

### Example 62: Labeling BSA with CAP3 in PIPES Buffer

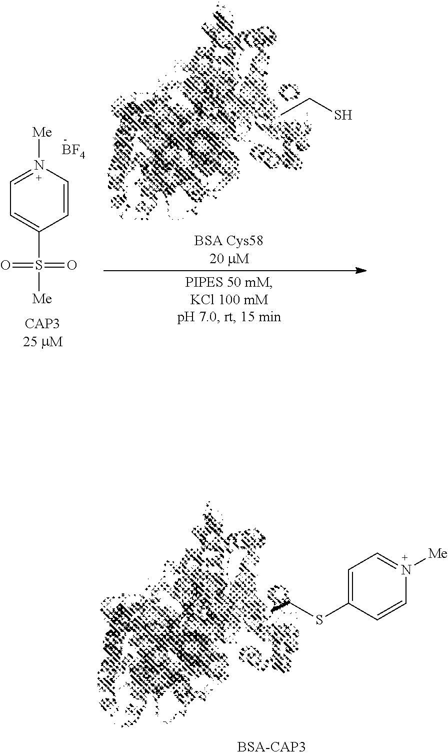

The stock solution of bovine serum albumin (BSA) in water (11.2 mg/mL, 169 μM, 3.56 μL) was mixed with PIPES buffer (24.94 μL, pH 7, 50 mM PIPES, 100 mM KCl). An aliquot of CAP3 in water (0.5 mM, 1.5 μL, 1.25 equiv.) was added to this mixture. The reaction was kept at room temperature for 15 min to generate a solution of BSA-CAP3. This sample was digested using Trypsin Singles, Proteomic Grade Kit (Sigma-Aldrich, Catalog Number: T7575) according to the manufacturer's directions. Briefly, trypsin solubilizing reagent (1 μL) was added to a trypsin singles vial. For native protein digestion, BSA (2 mg/mL, 30 μM, 20 μL) and tris buffer (pH 10.4, 20 μL) were combined in a microcentrifuge tube and transferred to the trypsin singles vial. For arylated protein digestion, the sample from the previous step was added to the respective trypsin singles vial. In both cases, trypsin reaction buffer (49 μL, 40 mM NH₄HCO₃ aq. and 9% v/v CH₃CN) was transferred to each vial. The digestion mixture was vortexed and centrifuged before a 5 h incubation at 37° C. The samples were acidified with 10% v/v CF₃CO₂H aq. (1.6 μL). The samples were diluted 1:10 with CH₃CN and analyzed by MALDI-TOF spectrometry with u-cyano-4-hydroxycinnamic acid (CHCA) matrix. Mass data were compared to mass lists generated from Expasy PeptideMass database (ID: ALBU_BOV) and indicated the formation of desired BSA-CAP3.

### Example 63: Labeling BSA with CAP4 in PIPES Buffer

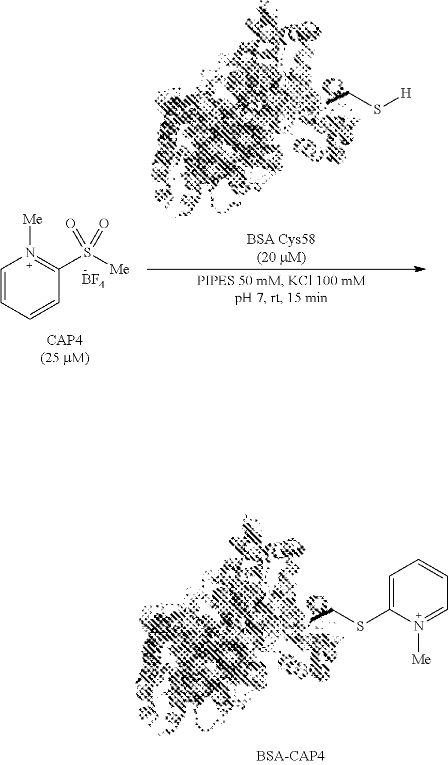

The stock solution of bovine serum albumin (BSA) in water (11.2 mg/mL, 169 μM, 3.56 μL) was mixed with PIPES buffer (24.94 μL, pH 7, 50 mM PIPES, 100 mM KCl). An aliquot of CAP4 in water (0.5 mM, 1.5 μL, 1.25 equiv.) was added to this mixture. The reaction was kept at room temperature for 15 min to generate a solution of BSA-CAP4. This sample was digested using Trypsin Singles, Proteomic Grade Kit (Sigma-Aldrich, Catalog Number: T7575) according to the manufacturer's directions. Briefly, trypsin solubilizing reagent (1 μL) was added to a trypsin singles vial. For native protein digestion, BSA (2 mg/mL, 30 μM, 20 μL) and tris buffer (pH 10.4, 20 μL) were combined in a microcentrifuge tube and transferred to the trypsin singles vial. For arylated protein digestion, the sample from the previous step was added to the respective trypsin singles vial. In both cases, trypsin reaction buffer (49 μL, 40 mM NH₄HCO₃ aq. and 9% v/v CH₃CN) was transferred to each vial. The digestion mixture was vortexed and centrifuged before a 5 h incubation at 37° C. The samples were acidified with 10% v/v CF₃CO₂H aq. (1.6 μL). The samples were diluted 1:10 with CH₃CN and analyzed by MALDI-TOF spectrometry with u-cyano-4-hydroxycinnamic acid (CHCA) matrix. Mass data were compared to mass lists generated from Expasy PeptideMass database (ID: ALBU_BOV) and indicated the formation of desired BSA-CAP4.

### Example 64: One-Pot Two-Step Labeling of BSA with CAP9 and Tetramethylrhodamine Phenol Derivative

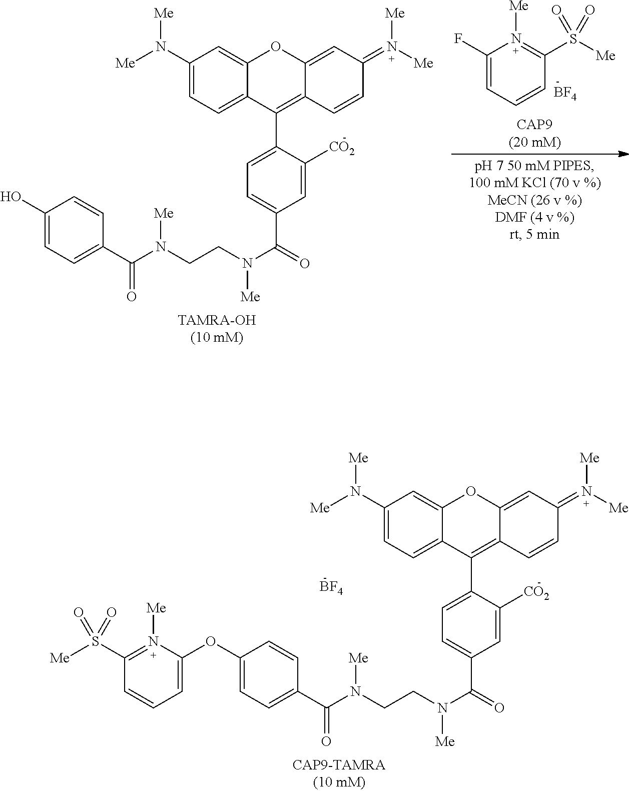

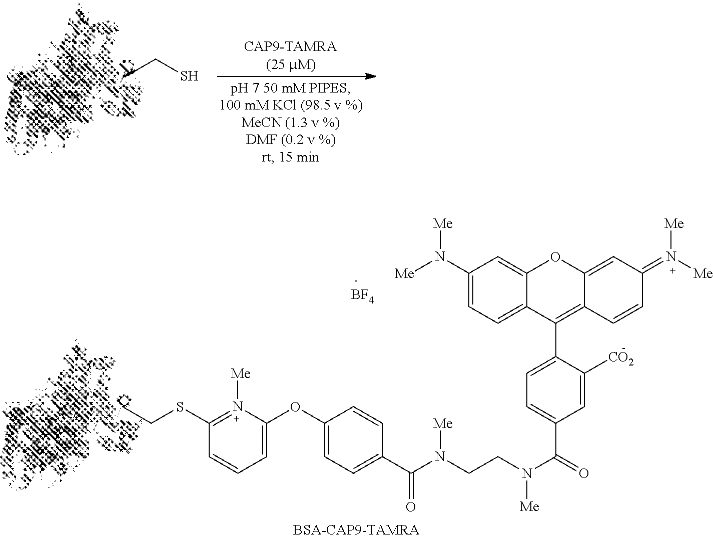

An aliquot of freshly prepared CAP9 (2 μL, 100 mM in MeCN) was added to a mixture of PIPES buffer (7 μL, pH 7, 50 mM PIPES, 100 mM KCl) and tetramethyl rhodamine phenol derivative (1 μL, 100 mM in MeCN/DMF, 63:37 v:v). The reaction was kept at room temperature for 5 min. An aliquot of this solution (1 μL, 100 mM) was diluted with water (19 μL) to give a 10 mM CAP9-TAMRA solution. An aliquot of the above CAP9-TAMRA solution (1.5 μL, 0.5 mM, 1.25 equiv.) was mixed with BSA (3.56 μL, 169 μM in water) in PIPES buffer (24.94 μL, pH 7, 50 mM PIPES, 100 mM KCl). After 15 min, the reaction mixture was analyzed by gel electrophoresis. Protein samples were loaded in 2× Laemmli sample buffer (Bio-Rad 1610737) and resolved by SDS-PAGE (Bio-Rad, Any kD™ mini-PROTEAN® TGX Stain-Free™ protein gels, 4568126). The gel was visualized with an Azure Sapphire Biomolecular Imager for TAMRA (excitation 520 nm; emission 565 nm), which confirmed the formation of the desired BSA-CAP9-TAMRA conjugate. Analogous results were obtained with CAP9-TAMRA prepared with CAP9 and the TAMRA derivative in 4:1 molar ratio under similar reaction conditions.

### Example 65: One-Pot Two-Step Labeling of GFP V150C with CAP9 and Tetramethyl Rhodamine Phenol Derivative

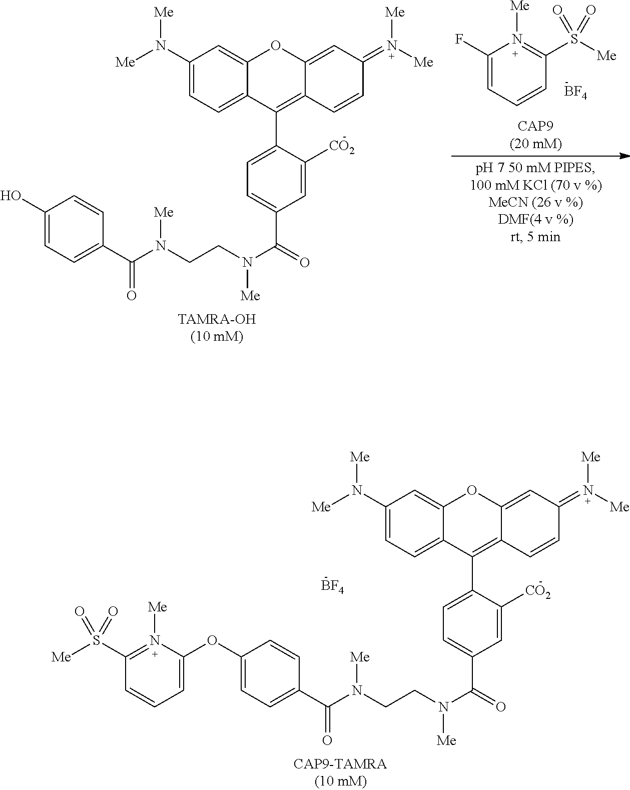

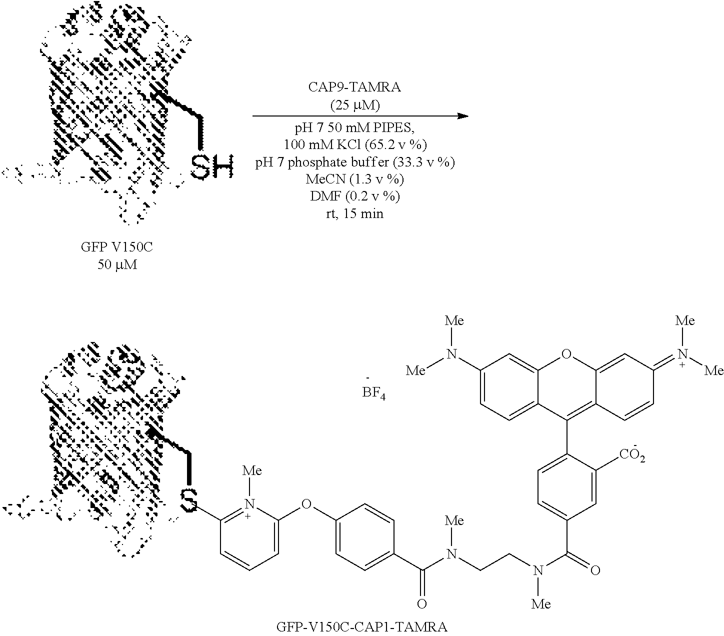

An aliquot of freshly prepared CAP9 (2 μL, 100 mM in MeCN) was added to a mixture of PIPES buffer (7 μL, pH 7, 50 mM PIPES, 100 mM KCl) and tetramethyl rhodamine phenol derivative (1 μL, 100 mM in MeCN/DMF, 63:37 v:v). The reaction was kept at room temperature for 5 min. An aliquot of this solution (1 μL, 100 mM) was diluted with water (19 μL) to give a 10 mM CAP9-TAMRA solution. An aliquot of the above CAP9-TAMRA solution (0.75 μL, 0.5 mM, 1.25 equiv.) was mixed with GFP V150C (5 μL, 60 μM in pH 7 phosphate buffer) in PIPES buffer (9.25 μL, pH 7, 50 mM PIPES, 100 mM KCl). After 15 min, the reaction mixture was analyzed by gel electrophoresis. Protein samples were loaded in 2× Laemmli sample buffer (Bio-Rad 1610737) and resolved by SDS-PAGE (Bio-Rad, Any kD™ mini-PROTEAN® TGX Stain-Free™ protein gels, 4568126). The gel was visualized with an Azure Sapphire Biomolecular Imager for GFP (excitation 488 nm; emission 518 nm) and TAMRA (excitation 520 nm; emission 565 nm), which confirmed the formation of the desired GFP-V150C-CAP9-TAMRA conjugate. Analogous results were obtained with CAP9-TAMRA prepared with CAP9 and TAMRA in 4:1 molar ratio under similar reaction conditions.

### Quantification of the Reactivity of CAP1-a Toward Various Nucleophiles

In a typical experiment, an aliquot of the stock solution of CAP1-a in water was added to nucleophile in PIPES buffer or water. The progress of the reaction was monitored by UV-Vis spectrometer at suitable wavelength. The pseudo first order reaction rate constant was determined by plotting the ln value of the concentration of CAP1-a against the reaction time. The pseudo first order reaction rate constant thus obtained was plotted against the concentration of the nucleophile. The slope of this linear correlation is the second order reaction rate constant. CAP1-a was reacted separately with GSH, cysteine, N-acetylcysteine, and phenol.

Unexpectedly, each reaction proceeded at an exceptionally high second order reaction rate constant (k₂). The reaction between CAP1-a and GSH had a k₂ of 1.2×10² M⁻¹·S⁻¹ at 25° C. and pH 7 in 50 mM PIPES. The k₂ for the reaction between CAP1-a and cysteine was 2.8×10² M⁻¹·S⁻¹ under similar reaction conditions. On the other hand, the k₂ for the reaction between CAP1-a and water was only 4.5×10⁻⁶ M⁻¹ S⁻¹, indicating the high stability of CAP1-a in water.

### Quantification of the Reactivity of CAP Compounds Toward Various Nucleophiles

Following the study above, compounds CAP1-F, CAP2, and CAP4 each were reacted with either GSH or phenol in water or 50 mM PIPES at pH 7. Surprisingly, each reaction proceeded at an exceptionally high second order reaction rate constant (k₂). The reaction between CAP4 and GSH had a k₂ as high as 5.3×10⁴ M⁻¹·S⁻¹ at 25° C. and pH 7 in 50 mM PIPES.

On the other hand, the reaction between CAP4 and water is very slow, showing a k₂ of only 4.8−10⁻⁶ M⁻¹·S⁻¹ at 25° C. and pH 7 in 50 mM PIPES. The slow reaction with water demonstrates that CAP compounds are stable in water.

In another study, compounds CAP1-b, CAP1-F, CAP2, CAP3, CAP4, and CAP5 each were reacted with either GSH in a 50 mM PIPES and 150 mM KCl aqueous solution at pH 7 and 25° C. Surprisingly, each reaction proceeded at a high rate. The reaction between CAP5 and GSH has a k₂ as high as 3.2×10⁵ M⁻¹·S⁻¹, 7×10⁹ folds faster than its reaction with water under the same conditions. Similarly, the reactions between GSH and one of CAP1-b, CAP1-F, CAP2, CAP3, and CAP4 had a k₂ of, respectively, 1.2×10² M⁻¹·S⁻¹, 3.5×10 M⁻¹·S⁻¹, 2.4×10 M⁻¹·S⁻¹, 9.8×10³ M⁻¹·S⁻¹, and 1.2×10⁵ M⁻¹. S⁻¹.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made and used for modifying a substrate having a suitable functional group. Thus, other embodiments were also within the claims.

What is claimed is:

1. A pnictogen-containing heterocyclic compound of Formula (I): 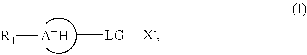
in which
R₁ is C₁-C₆ alkyl, C₄-C₁₀ cycloalkyl, 5- to 8-membered heterocycloalkyl, aryl, or heteroaryl; 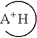
is a heterocyclic ring;
A⁺ is a cationic quaternary pnictogen atom;
LG is a leaving group selected from the group consisting of halogen, aryloxy, RₐO₂S—, R_{b}O₂S—O—, R_{c}OS—, R_{d}S(O)(NSO₂Rₑ)—, and R_{f}S(O)(N⁺(CH₃)₂)—, each of Rₐ, R_{b}, R_{c}, R_{d}, Rₑ, and R_{f}, independently, being C₁-C₆ alkyl, aryl, heteroaryl, halogen, alkoxy, or aryloxy;
X⁻ is a counter anion selected from the group consisting of halide, polyhalide anion, perchlorate, hydroxide, peroxide, siloxide, sulfate, hydrogen sulfate, sulfite, disulfite, dithionate, dithionite, halosulfate, thiosulfate, persulfate, disulfate, sulfinate (R′SO₂), sulfonate (R″SO₃⁻), bis(sulfonyl)imide ((R′SO₂)₂N⁻), nitrate, nitrite, azide, cyanide, cyanate, thiocyanate, phosphate, metaphosphate, polyphosphate, hydrogen phosphate, dihydrogen phosphate, monohalophosphate, dihalophosphate, hexahalophosphate, organophosphonate (R′P(O)(O⁻)₂ or R′P(O)(OR″)(O⁻)), organophosphate (R′OP(O)(O⁻)₂ or R′OP(O)(OR″)(O⁻)), arsenate, alkoxide, alkenoxide, aryloxide, carboxylate, percarboxylate, carbonate, bicarbonate, oxalate, borate, tetrahaloborate, tetraalkylborate, tetraarylborate, carborane, and combinations thereof, each of R′ and R″, independently, being C₁-C₆ alkyl or aryl; and
the number of net negative charges of X⁻ equals to that of the net positive charges of

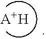

2. The pnictogen-containing heterocyclic compound of claim 1, wherein the compound is a compound of Formula (II): 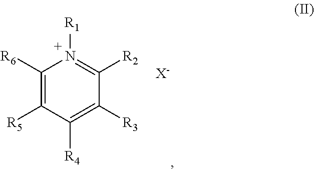
in which
the cationic quaternary pnictogen atom is N⁺ in Formula (II);
R₁ is CH₃, CH₂CH₃ or -L-Het⁺;
each of R₂, R₃, R₄, R₅, and R₆, independently, is H, F, Cl, Br, I, C₁-C₁₀ alkoxy, RₐO₂S—, R_{b}O₂S—O—, R_{f}S(O)(N⁺(CH₃)₂)—, halogenated aryl, halogenated heteroaryl, Het⁺, or -L-Het⁺, in which Het⁺ is 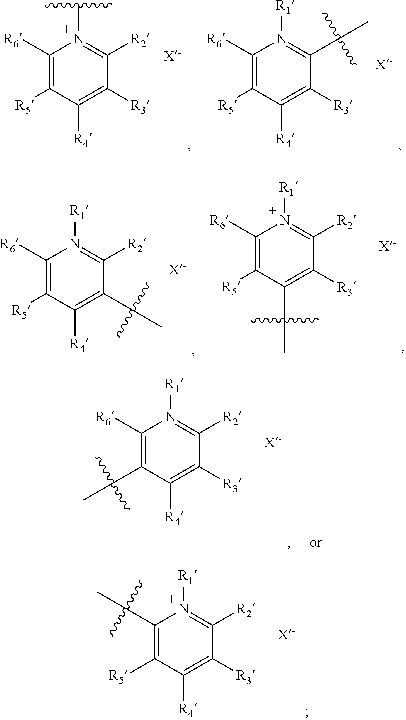
R₁′ is CH₃ or CH₂CH₃; each of R₂′, R₃′, R₄′, R₅′, and R₆′, independently, is H, NO₂, F, Cl, Br, I, Rₐ′O₂S—, R_{b}′O₂S—O—, or R_{f}S(O)(N⁺(CH₃)₂)—; each of Rₐ′, R_{b}′, and R_{f}′, independently, is C₁-C₆ alkyl or aryl; and X′⁻ is a counter anion selected from the group consisting of halide, polyhalide anion, perchlorate, hydroxide, peroxide, siloxide, sulfate, hydrogen sulfate, sulfite, disulfite, dithionate, dithionite, halosulfate, thiosulfate, persulfate, disulfate, sulfinate (R′SO₂⁻), sulfonate (R″SO₃⁻), bis(sulfonyl)imide ((R′SO₂)₂N⁻), nitrate, nitrite, azide, cyanide, cyanate, thiocyanate, phosphate, metaphosphate, polyphosphate, hydrogen phosphate, dihydrogen phosphate, monohalophosphate, dihalophosphate, hexahalophosphate, organophosphonate (R′P(O)(O⁻)₂ or R′P(O)(OR″)(O⁻)), organophosphate (R′OP(O)(O⁻)₂ or
R′OP(O)(OR″)(O⁻)), arsenate, alkoxide, alkenoxide, aryloxide, carboxylate, percarboxylate, carbonate, bicarbonate, oxalate, borate, tetrahaloborate, tetraalkylborate, tetraarylborate, carborane, and combinations thereof, each of R′ and R″, independently, being C₁-C₆ alkyl or aryl; L is a linker selected from the group consisting of 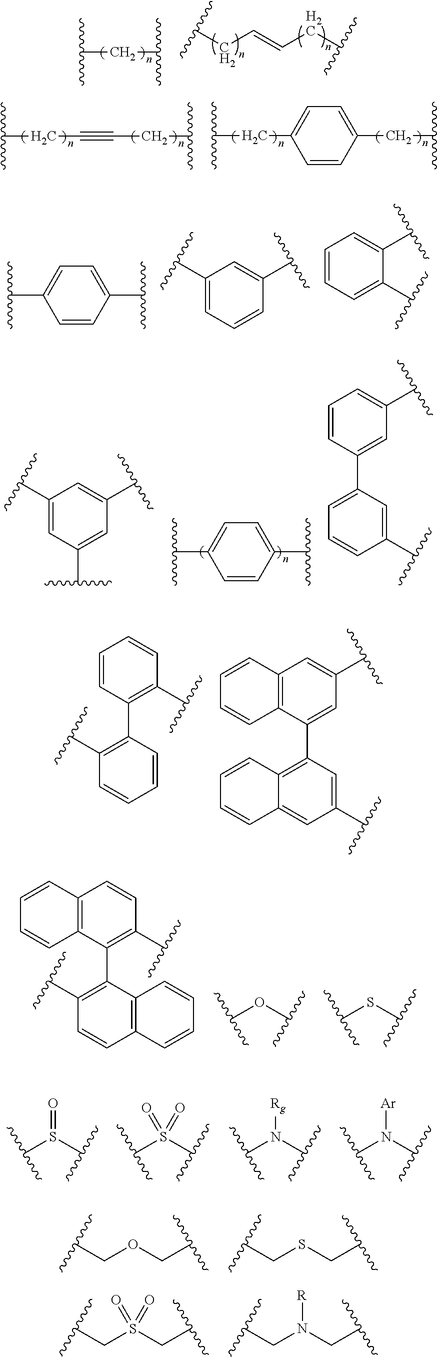 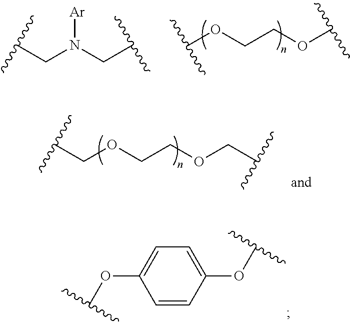
n is an integer from 0 to 20;
R_{g} is C₁-C₆ alkyl; and Ar is aryl;
at least one of R₂, R₃, R₄, R₅, and R₆ is the leaving group that is F, Cl, Br, I, RₐO₂S—, or R_{b}O₂S—O—;
when R₂ is F, (i) R₄ is F, Cl, Br, I, C₁-C₁₀ alkoxy, RₐO₂S—, or R_{b}O₂S—O—, (ii) R₅ is NO₂, or (iii) one and only one of R₃, R₄, R₅, and R₆ is Het⁺;
when R₂ is Cl, R₅ is H or Het⁺; and
when R₄ is —SO₂CH₃, (i) R₂ is F, Cl, Br, I, C₁-C₁₀ alkoxy, RₐO₂S—, or R_{b}O₂S—O—, or (ii) one and only one of R₂, R₃, R₅, and R₆ is Het⁺.

3. The pnictogen-containing heterocyclic compound of claim 2, wherein at least one of R₂, R₄, and R₆ is the leaving group.

4. The pnictogen-containing heterocyclic compound of claim 3, wherein the leaving group is F or CH₃O₂S—.

5. The pnictogen-containing heterocyclic compound of claim 2, wherein R₂, being the leaving group, is CH₃O₂S—, and R₄ is H, C₁-C₁₀ alkoxy, or Het⁺.

6. The pnictogen-containing heterocyclic compound of claim 2, wherein R₁ is methyl or ethyl.

7. The pnictogen-containing heterocyclic compound of claim 2, wherein R₃ or R₅ is NO₂.

8. The pnictogen-containing heterocyclic compound of claim 2, wherein the compound is a compound of Formula (III): 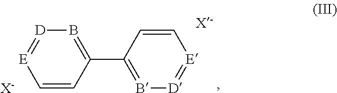
in which
one and only one of B, D, and E is N⁺CH₃ or N⁺CH₂CH₃;
one and only one of B, D, and E is CR₂, R₂ being F, Cl, Br, I, RₐO₂S—, or R_{b}O₂S—O—;
one and only one of B, D, and E is CR₁₁, R₁₁ being C₁-C₆ alkyl, C₁-C₆ alkoxy, C₂-C₆ alkenyl, C₂-C₆ alkynyl, C₁-C₆ alkyl amino, C₄-C₁₀ cycloalkyl, C₄-C₁₀ cycloalkoxy, 5- to 8-membered heterocycloalkyl, 5- to 8-membered heterocycloalkoxy, aryl, aryloxy, heteroaryl, or heteroaryloxy;
one and only one of B′, D′, and E′ is N⁺CH₃ or N⁺CH₂CH₃;
one and only one of B′, D′, and E′ is CR₂′, R₂′ being F, Cl, Br, I, Rₐ′O₂S—, or R_{b}′O₂S—O—;
one and only one of B′, D′, and E′ is CR₁₁′, R₁₁′ being C₁-C₆ alkyl, C₁-C₆ alkoxy, C₂-C₆ alkenyl, C₂-C₆ alkynyl, C₁-C₆ alkyl amino, C₄-C₁₀ cycloalkyl, C₄-C₁₀ cycloalkoxy, 5- to 8-membered heterocycloalkyl, 5- to 8-membered heterocycloalkoxy, aryl, aryloxy, heteroaryl, or heteroaryloxy; and
X′⁻ is a counter anion selected from the group consisting of halide, polyhalide anion, perchlorate, hydroxide, peroxide, siloxide, sulfate, hydrogen sulfate, sulfite, disulfite, dithionate, dithionite, halosulfate, thiosulfate, persulfate, disulfate, sulfinate (R′SO₂⁻), sulfonate (R″SO₃⁻), bis(sulfonyl)imide ((R′SO₂)₂N⁻), nitrate, nitrite, azide, cyanide, cyanate, thiocyanate, phosphate, metaphosphate, polyphosphate, hydrogen phosphate, dihydrogen phosphate, monohalophosphate, dihalophosphate, hexahalophosphate, organophosphonate (R′P(O)(O⁻)₂ or R′P(O)(OR″)(O⁻)), organophosphate (R′OP(O)(O⁻)₂ or
R′OP(O)(OR″)(O⁻)), arsenate, alkoxide, alkenoxide, aryloxide, carboxylate, percarboxylate, carbonate, bicarbonate, oxalate, borate, tetrahaloborate, tetraalkylborate, tetraarylborate, carborane, and combinations thereof, each of R′ and R″, independently, being C₁-C₆ alkyl or aryl.

9. The pnictogen-containing heterocyclic compound of claim 8, wherein each of R₂ and R₂′, independently, is F or CH₃O₂S—.

10. The pnictogen-containing heterocyclic compound of claim 1, wherein the compound is a compound of Formula (IV): 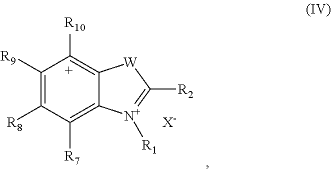
in which
R₁ is CH₃ or CH₂CH₃;
R₂ is RₐO₂S— or R_{b}O₂S—O—;
each of R₇, R₅, R₉, and R₁₀, independently, is H or C₁-C₆ alkyl; and
W is S or NR₁₁, R₁₁ being C₁-C₆ alkyl.

11. The pnictogen-containing heterocyclic compound of claim 10, wherein R₂ is CH₃O₂S—.

12. The pnictogen-containing heterocyclic compound of claim 1, wherein the compound is a compound of Formula (V): 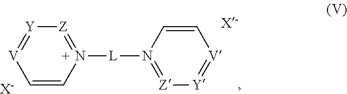
in which
one and only one of V, Y, and Z is CR₂, R₂ being F, Cl, Br, I, RₐO₂S—, or R_{b}O₂S—O—;
the other two of V, Y, and Z is CR₁₁, R₁₁ being C₁-C₆ alkyl, C₁-C₆ alkoxy, C₂-C₆ alkenyl, C₂-C₆ alkynyl, C₁-C₆ alkyl amino, C₄-C₁₀ cycloalkyl, C₄-C₁₀ cycloalkoxy, 5- to 8-membered heterocycloalkyl, 5- to 8-membered heterocycloalkoxy, aryl, aryloxy, heteroaryl, or heteroaryloxy;
one and only one of V′, Y′, and Z′ is CR₂′, R₂′ being F, Cl, Br, I, Rₐ′O₂S—, or R_{b}′O₂S—O—;
the other two of V′, Y′, and Z′ is CR₁′, R₁₁′ being C₁-C₆ alkyl, C₁-C₆ alkoxy, C₂-C₆ alkenyl, C₂-C₆ alkynyl, C₁-C₆ alkyl amino, C₄-C₁₀ cycloalkyl, C₄-C₁₀ cycloalkoxy, 5- to 8-membered heterocycloalkyl, 5- to 8-membered heterocycloalkoxy, aryl, aryloxy, heteroaryl, or heteroaryloxy;
L is a linker selected from the group consisting of 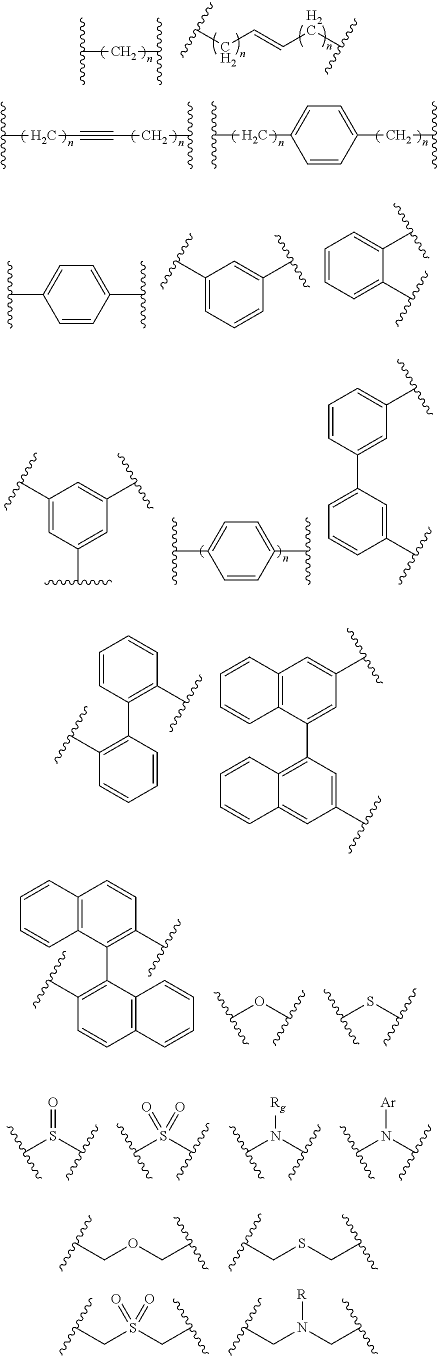 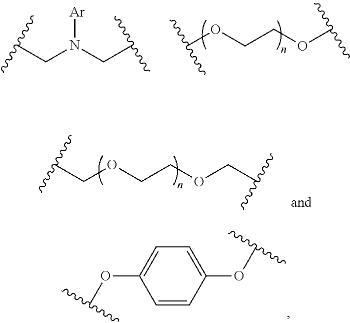
n being an integer from 0 to 20, R_{g} is C₁-C₆ alkyl, and Ar is aryl; and
X⁻ is a counter anion selected from the group consisting of halide, polyhalide anion, perchlorate, hydroxide, peroxide, siloxide, sulfate, hydrogen sulfate, sulfite, disulfite, dithionate, dithionite, halosulfate, thiosulfate, persulfate, disulfate, sulfinate (R′SO₂⁻), sulfonate (R″SO₃⁻), bis(sulfonyl)imide ((R′SO₂)₂N⁻), nitrate, nitrite, azide, cyanide, cyanate, thiocyanate, phosphate, metaphosphate, polyphosphate, hydrogen phosphate, dihydrogen phosphate, monohalophosphate, dihalophosphate, hexahalophosphate, organophosphonate (R′P(O)(O⁻)₂ or R′P(O)(OR″)(O⁻)), organophosphate (R′OP(O)(O⁻)₂ or
R′OP(O)(OR″)(O⁻)), arsenate, alkoxide, alkenoxide, aryloxide, carboxylate, percarboxylate, carbonate, bicarbonate, oxalate, borate, tetrahaloborate, tetraalkylborate, tetraarylborate, carborane, and combinations thereof, each of R′ and R″, independently, being C₁-C₆ alkyl or aryl.

13. The pnictogen-containing heterocyclic compound of claim 1, wherein the compound is selected from the group consisting of:

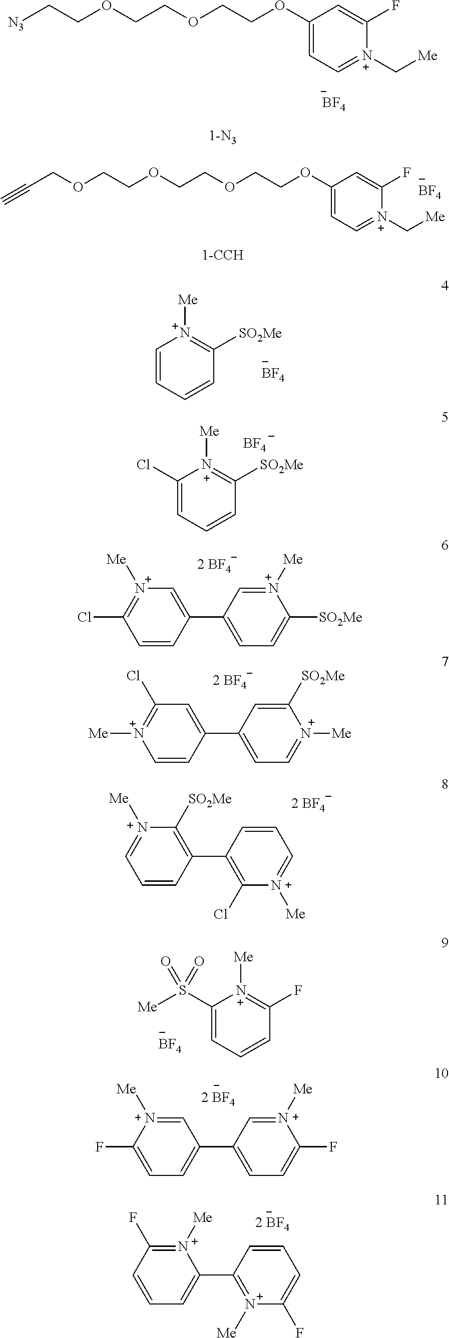

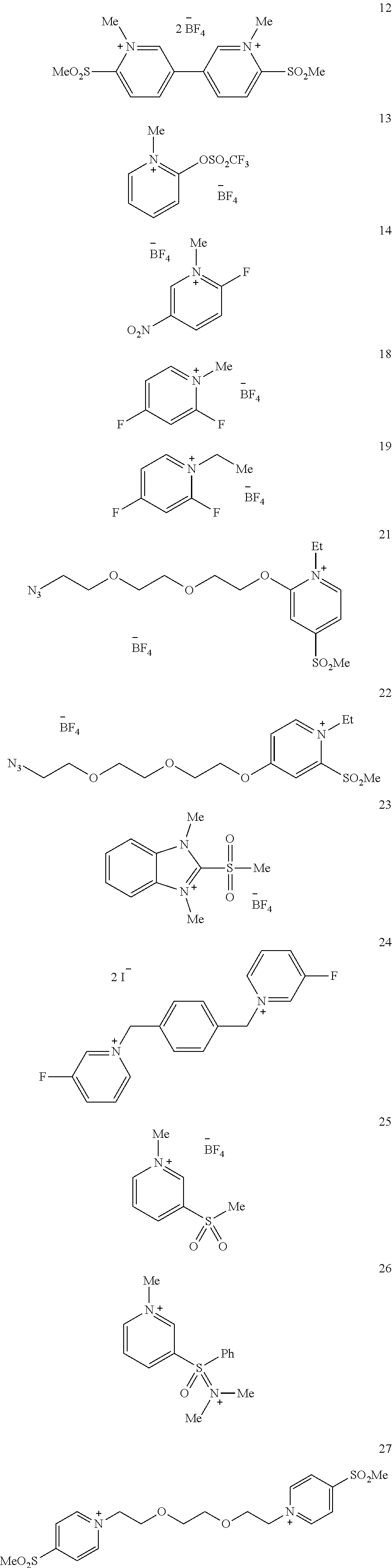

14. The pnictogen-containing heterocyclic compound of claim 13, wherein the compound is one of Compounds 4-9, 21, and 22.

15. A method for modifying a substrate, the method comprising the steps of:
a) providing a substrate, and
b) contacting the substrate with a pnictogen-containing heterocyclic compound of claim 1, thereby obtaining a conjugated substrate containing a substrate moiety derived from the substrate and a pnictogen-containing heterocyclic moiety derived from the pnictogen-containing heterocyclic compound.
wherein
the substrate contains a functional group selected from the group consisting of hydroxyl, thiol, selenol, and tellurol, and
the substrate moiety is linked to the pnictogen-containing heterocyclic moiety via a covalent bond.

16. The method of claim 15, wherein the pnictogen-containing heterocyclic compound is a compound of Formula (II): 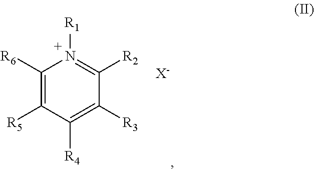
in which
R₁ is CH₃, CH₂CH₃, or -L-Het⁺;
each of R₂, R₃, R₄, R₅, and R₆, independently, is H, F, Cl, Br, I, C₁-C₁₀ alkoxy, RₐO₂S—, R_{b}O₂S—O—, Het⁺, or -L-Het⁺, in which Het⁺ is 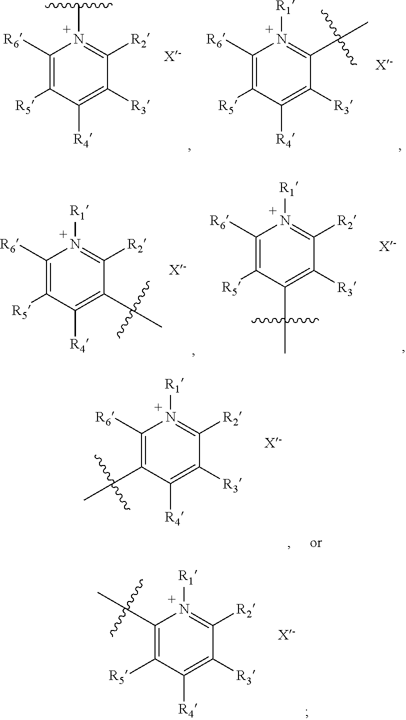
L is a linker selected from the group consisting of 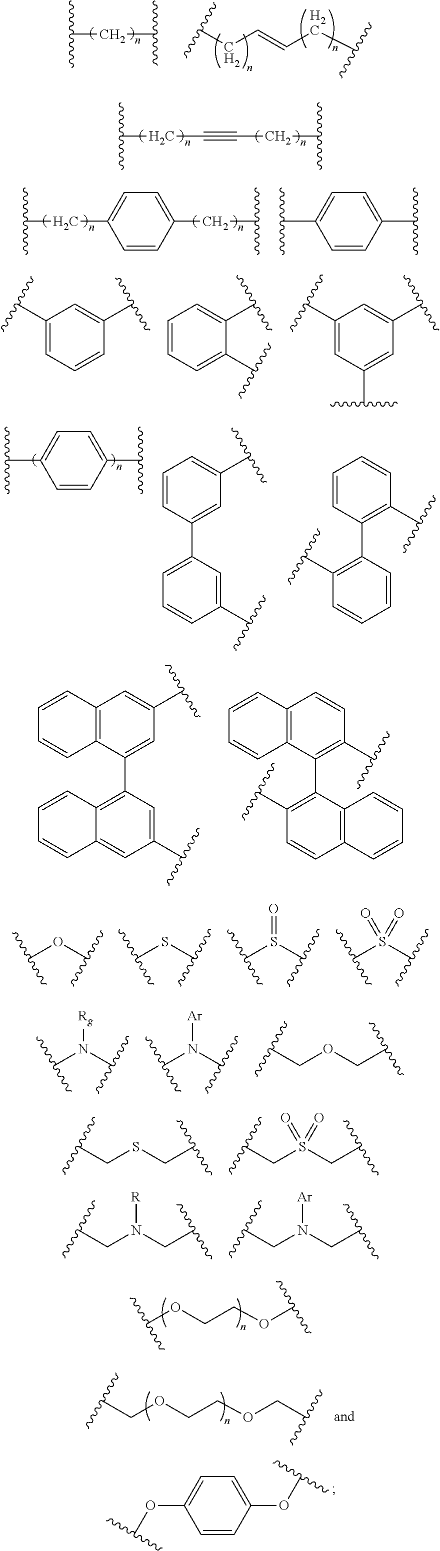
n is an integer from 0 to 20; R_{g} is C₁-C₆ alkyl; Ar is aryl; R₁′ is CH₃ or CH₂CH₃; each of R₂′, R₃′, R₄′, R′, and R₆′, independently, is H, NO₂, F, Cl, Br, I, Rₐ′O₂S—, or R_{b}′O₂S—O—; each of Rₐ′ and R_{b}′, independently, is C₁-C₆ alkyl; and X′⁻ is a counter anion selected from the group consisting of halide, polyhalide anion, perchlorate, hydroxide, peroxide, siloxide, sulfate, hydrogen sulfate, sulfite, disulfite, dithionate, dithionite, halosulfate, thiosulfate, persulfate, disulfate, sulfinate (R′SO₂⁻), sulfonate (R″SO₃⁻), bis(sulfonyl)imide ((R′SO₂)₂N⁻), nitrate, nitrite, azide, cyanide, cyanate, thiocyanate, phosphate, metaphosphate, polyphosphate, hydrogen phosphate, dihydrogen phosphate, monohalophosphate, dihalophosphate, hexahalophosphate, organophosphonate (R′P(O)(O⁻)₂ or R′P(O)(OR″)(O⁻)), organophosphate (R′OP(O)(O⁻)₂ or R′OP(O)(OR″)(O⁻)), arsenate, alkoxide, alkenoxide, aryloxide, carboxylate, percarboxylate, carbonate, bicarbonate, oxalate, borate, tetrahaloborate, tetraalkylborate, tetraarylborate, carborane, and combinations thereof, each of R′ and R″, independently, being C₁-C₆ alkyl or aryl;
at least one of R₂, R₃, R₄, R₅, and R₆ is the leaving group that is F, Cl, Br, I, RₐO₂S—, or R_{b}O₂S—O—.

17. The method of claim 15, wherein the contacting step is performed in a medium, and the substrate is a biomolecule selected from the group consisting of an amino acid, a peptide, a protein, a deoxyribonucleic acid or a derivative thereof, a ribonucleic acid or a derivative thereof, a peptoid, and poly(amino acid).

18. A conjugated biomolecule comprising:
(i) a biomolecule moiety derived from a biomolecule, and
(ii) a pnictogen-containing heterocyclic moiety derived from a pnictogen-containing heterocyclic compound of claim 1,
wherein
the biomolecule is an amino acid, a peptide, a protein, a deoxyribonucleic acid or a derivative thereof, a ribonucleic acid or a derivative thereof, a peptoid, or poly(amino acid), each having a functional group selected from the group consisting of hydroxyl, thiol, selenol, and tellurol, and
the biomolecule moiety is linked to the pnictogen-containing heterocyclic moiety via a covalent bond formed by substitution of the leaving group in the pnictogen-containing heterocyclic compound by the functional group in the biomolecule.

19. The conjugated biomolecule of claim 18, further comprising a therapeutic moiety derived from a therapeutic agent selected from the group consisting of an anticancer agent, a therapeutically active peptide and protein, a medication for a neurological disorder, a molecular scaffold for targeted drug delivery, or a radioactive tracer, wherein the therapeutic moiety is linked to the pnictogen-containing heterocyclic moiety via a covalent bond, the therapeutic agent contains a functional group selected from the group consisting of hydroxyl, thiol, selenol, and tellurol, and the pnictogen-containing heterocyclic compound has two leaving groups.

20. A pharmaceutical composition comprising a conjugated biomolecule of claim 18 and a pharmaceutically acceptable carrier thereof.
